(19) United States
(12) Patent Application Publication
(10) Pub. No.: US 2018/0356429 A1
(43) Pub. Date: Dec. 13, 2018

(54) METHODS OF DETECTING AND QUANTIFYING IL-13 AND USES IN DIAGNOSING AND TREATING TH2-ASSOCIATED DISEASES

(71) Applicants: Genentech, Inc., South San Francisco, CA (US); Hoffmann-La Roche Inc., Little Falls, NJ (US)

(72) Inventors: Alyssa Morimoto, South San Francisco, CA (US); Kun Peng, South San Francisco, CA (US); Heleen Scheerens, South San Francisco, CA (US); Fang Cai, South San Francisco, CA (US); Hans Hornauer, Peissenberg (DE)

(73) Assignees: Genentech, Inc., South San Francisco, CA (US); Hoffmann-La Roche Inc., Little Falls, NJ (US)

(21) Appl. No.: 15/700,790

(22) Filed: Sep. 11, 2017

Related U.S. Application Data
(63) Continuation of application No. PCT/US2016/022481, filed on Mar. 15, 2016.
(60) Provisional application No. 62/133,693, filed on Mar. 16, 2015.

(51) Int. Cl. G01N 33/68

(52) U.S. Cl. CPC G01N 33/6869; G01N 2333/5437; G01N 2800/56; G01N 2800/122; G01N 2800/52

(57) ABSTRACT

Methods of detecting and quantifying IL-13 are provided. Also provided are methods of diagnosing, selecting and identifying patients with Th2-associated diseases (Type 2-associated diseases) for treatment with certain therapeutic agents that are Th2 pathway inhibitors (Type 2 pathway inhibitors).

### CROSS REFERENCE TO RELATED APPLICATION

This application is a continuation of International Application No. PCT/US2016/022481 having an international filing date of Mar. 15, 2016, which claims the benefit of priority of provisional U.S. Application No. 62/133,693 filed Mar. 16, 2015, both of which are hereby incorporated by reference in their entirety.

### SEQUENCE LISTING

The instant application contains a Sequence Listing which has been submitted via EFS-Web and is hereby incorporated by reference in its entirety. Said ASCII copy, created on Sep. 5, 2017, is named P32675-US-1_SL.txt and is 20,348 bytes in size.

### FIELD

Methods of detecting and quantifying IL-13 are provided. Also provided are methods of diagnosing, selecting and identifying patients with Th2-associated diseases for treatment with certain therapeutic agents that are Th2 pathway inhibitors.

### BACKGROUND

Interleukin (IL)-13 is considered a key mediator of T-helper type 2 (Th2) inflammation and elevated levels of IL-13 have been associated with numerous diseases including, but not limited to, asthma, inflammatory bowel disease, idiopathic pulmonary fibrosis (IPF), chronic obstructive pulmonary disease (COPD) and atopic dermatitis and others (Oh C K, et al., Eur Respir Rev 19:46-54 (2010); Fahy J V, et al., Nat Rev Immunol 15:57-65 [2015]). IL-13 is produced by many cell types, including Th2 cells, basophils, eosinophils, and mast cells, as well as airway epithelial cells and Type 2 innate lymphoid cells. IL-13 binds to a heterodimeric receptor, IL-4Rα/IL-13Rα1 that is shared with IL-4 and activates the STAT-6 signaling pathway (Hershey G K, J Allergy Clin Immunol 111(4):677-90 [2003]). It has been associated with clinical manifestations of asthma in certain cases including mucus production, subepithelial fibrosis, IgE production, smooth muscle hyperplasia, as well as inflammatory cell recruitment and activation (Hershey G K, J Allergy Clin Immunol 111(4):677-90 [2003]; Fahy J V, et al., Nat Rev Immunol 15:57-65 [2015]). Because Th2 inflammation involves the activity of several cell types in addition to Th2 cells, including Type 2 innate lymphoid cells (ILC2s), “Th2 inflammation” has more recently been referred to in the scientific literature as “Type 2 inflammation.” In addition to Th2 cells, ILC2s have been identified as important sources of cytokines such as IL-5 and IL-13. Accordingly, cytokines such as IL-13 and IL-5 that have been previously identified as Th2 cytokines are now also referred to as Type 2 cytokines in the scientific literature. Likewise, the disease states associated with such cytokines are now also referred to as Type 2-driven diseases or Type 2-associated diseases. See, e.g., Noonan et al., J. Allergy Clin Immunol., 132(3): 567-574 (2013); Hanania et al., Thorax 70(8): 748-56 (2015); and Cai et al., Bioanalysis 8(4): 323-332 (2016). For example, the use of the term “Type 2 asthma” in the scientific literature reflects an evolution in the understanding of asthma, and is characterized by high levels of interleukins including IL-5 and IL-13 in the lung tissue. Accordingly, “Th2” and “Type 2” are used interchangeably herein.

IPF is a specific form of fibrosing interstitial pneumonia of unknown etiology, limited to the lung and is characterized by varying degrees of interstitial fibrosis (Raghu G, et al., Am J Respir Crit Care Med 183:788-824 [2011]). Multiple observations support a role of IL-13 in IPF pathology (Zhu Z, et al., J Clin Invest 103:779-88 [1999]; Lee C G, et al., J Exp Med 194:809-22 [2001]; Park S W, et al., J Korean Med Sci 24:614-20 [2009]; Chandriani S, et al., J Immunol 193:111-9 [2014]). The expression of IL-13 and IL-13 receptors are increased in the lung tissue and bronchoalveolar lavage fluid (BAL) from patients with IPF relative to healthy controls (Jakubzick C, et al., Am J Pathol 164:1989-2001 [2004]; Park S W, et al., J Korean Med Sci 24:614-20 [2009]).

Another Th2 inflammation-associated disease with IL-13 as a key pathogenetic component is atopic dermatitis (AD). Increased expression of IL-13 has consistently been reported in AD skin (Hamid Q, et al., J Allergy Clin Immunol 98:225-31 [1996]; Jeong C W, et al., Clin Exp Allergy 33:1717-24 [2003]; Tazawa T, et al., Arch Dermatol Res 295:459-64 [2004]; Neis M M, et al., J Allergy Clin Immunol 118:930-7 [2006]; Suárez-Fariñas M, et al., J Allergy Clin Immunol 132:361-70 [2013]; Choy D F, et al., J Allergy Clin Immunol. 130:1335-43 [2012]) and some reports suggest a relationship between IL-13 expression and the severity of disease (La Grutta S, et al., Allergy 60:391-5 [2005]). IL-13 and its receptors have therefore become therapeutic targets for the treatment of asthma, IPF and AD, as well as other Th2-associated diseases (Corren J, et al., N Eng J Med 365:1088-98 [2011]; Scheerens H, et al., Clin Exp Allergy 44:38-46 [2014]; Beck L A, et al., N Eng J Med 371:130-9 [2014]).

IL-13 has been detected at the sites of action of asthma, IPF and AD, including bronchial biopsy, lung biopsy, induced sputum, BAL, nasal lavage fluid, nasopharyngeal aspirates, and skin biopsy. The results demonstrated elevated IL-13 levels in patients with Th2 inflammation-associated diseases and that such elevated IL-13 levels distinguished those patients from healthy controls (Fitzpatrick A M, et al., J Allergy Clin Immunol 125; 851-7.e18 [2010]; Becker A B, J Allergy Clin Immunol 109; S533-8 [2002]; Jakubzick C, et al., Am J Pathol 164: 1989-2001 [2004]; Noah T L, et al., Ann Allergy Asthma Immunol 96:304-10 [2006]; Feleszko W, et al., J Allergy Clin Immunol 117:97-102 [2006]; Eickmeier O, et al., Cytokine 50:152-157 [2010]). Direct airway and skin sampling, however, require invasive or inconvenient collection procedures. In contrast, peripheral blood is an easily accessible tissue and collection of it is a less invasive process. Because circulating levels of IL-13 are typically low and because of the difficulty in measuring these low levels, there is a lack of understanding of how circulating IL-13 levels relate to the levels at the sites of disease action. Accordingly, a highly sensitive and highly specific serum IL-13 assay is needed to characterize circulating IL-13 levels in Th2-driven diseases to facilitate our understanding of IL-13 contributions to disease mechanisms.

A plethora of drugs are on the market or in development for treating asthma and other Th-2 associated diseases. Targets for asthma and Th2-associated diseases include cytokines such as IL-13, IL-17, IL-5, and IL-4, and their receptors, as well as targets associated with allergy such as IgE. Exemplary therapeutic molecules on the market and therapeutic candidates in development for the treatment of asthma include, but are not limited to, omalizumab (XOLAIR®) (targeting soluble IgE) (see, e.g., Chang et al., J Allergy Clin Immunol. 117 (6): 1203-12 (2006); Winchester et al., N. Engl. J. Med. 355 (12): 1281-2 (2006); Brodlie et al., Arch Dis Child. 97 (7): 604-9 (2012); Bousquet et al., Chest 125 (4): 1378-86 (2004); Schulman, E S, Am J Respir Crit Care Med. 164 (8 Pt 2): S6-11 (2001); Chang et al., Adv Immunol. 93: 63-119 (2007)), lebrikizumab (targeting IL-13) (see, e.g., Corren et al. (2011) N Engl J Med 365: 1088-98; Scheerens et al. (2012) Am J Respir Crit Care Med 185: A3960; Jia et al. (2012) J Allergy Clin Immunol 130: 647-654 e10; WO 2012/083132), mepolizumab (targeting IL-5) (see, e.g., Haldar et al., N Engl J Med. 2009 Mar. 5; 360(10):973-84; Nair et al., N Engl J Med. 2009 Mar. 5; 360(10):985-93; Pavord et al., The Lancet 380 (9842): 651-659, doi:10.1016/S0140-6736(12)60988-X), quilizumab (targeting membrane-bound IgE) (see, e.g., US patent pub. no. 2013/0243750) and dupilimab (targeting IL-4Rα receptor) (see, e.g., Beck L A, et al., N Eng J Med 371:130-9 [2014]).

Although human asthma is commonly regarded as an allergic disorder characterized by type 2 cytokine expression and eosinophilic inflammation in the airways, it is clearly heterogeneous with respect to airway inflammation. Genomic approaches have identified heterogeneous gene expression patterns in asthmatic airways corresponding to the degree of type 2 cytokine expression and eosinophilic inflammation. These gene expression patterns have led to the identification of candidate biomarkers of eosinophilic airway inflammation that do not require bronchoscopy or sputum induction. See, e.g., WO 2009/124090, WO 2012/083132, and PCT/US2014/061759. Candidate biologic therapies targeting mediators of type 2 airway inflammation have progressed through clinical studies in patients with moderate-severe asthma in recent years. Serum periostin, fractional exhaled nitric oxide (FE_{NO}), and blood eosinophil counts are among those biomarkers that have emerged as potential predictive and pharmacodynamics biomarkers that may enrich for clinical benefit in clinical studies of biologic therapies targeting IL-13, IL-5, and IgE. Anon et al., 2013, DOI: 10.1513/AnnalsATS.201303-047AW.

Although such biomarkers as discussed above have demonstrated potential for identifying asthma patients that may be more likely to respond to particular therapeutic treatments, to date none have been validated and approved for such use by regulatory authorities. In addition, the previously identified biomarkers may have certain practical limitations and confounding factors associated with their use such as a need for a particular device to measure the biomarker, significant intrapatient or interpatient variability, or biomarker levels that may vary during development (e.g., pediatric levels compared to adult levels) or that may vary with concomitant medications. Also, no clinically validated diagnostic markers, e.g., biomarkers, have been identified that enable clinicians or others to accurately define pathophysiological aspects of asthma and other Th2-associated diseases, clinical activity, predict response to therapy, prognosis, or risk of developing the disease. Accordingly, as asthma patients and patients with Th2-associated diseases seek treatment, there is at present considerable trial and error involved in the search for therapeutic agent(s) effective for a particular patient. Such trial and error often involves considerable risk and discomfort the patient in order to find the most effective therapy.

Thus, there is a continuing need to identify new biomarkers that are effective for determining which asthma patients, and patients suffering with other Th2-associated diseases such as, for example but not limited to, atopic dermatitis, allergic rhinitis, nasal polyposis, eosiniophilic esophagitis, hypereosinophilic syndrome, COPD, or IBD, will respond to which treatment and for incorporating such determinations into more effective treatment regimens for asthma and other Th2-associated-disease patients. In addition, statistically and biologically significant and reproducible information regarding associations of such biomarkers with disease state could be utilized as an integral component in efforts to identify specific subsets of patients who would be expected to significantly benefit from treatment with a particular therapeutic agent, for example, where the therapeutic agent is or has been shown in clinical studies to be of therapeutic benefit in such specific patient subpopulation.

As mentioned above, circulating levels (serum levels) of IL-13 are typically low and therefore, difficult to measure with currently available methods. Currently available methods include a number of different immunoassay methods such as commercially available enzyme-linked immunosorbent assays (ELISA) and bead-based multiplex assays, including two assays that use platforms described as ultrasensitive, the Erenna® platform from Singulex® (Alameda, Calif.) and the Simoa™ platform from Quanterix™ (Lexington, Mass.) (Fischer et al., The AAPS Journal 17:93-101 [2015]). Using such assays, the scientific literature reports a wide range of circulating IL-13 levels for atopic individuals, asthmatic individuals, and healthy controls with reports ranging from low pg/mL to sub-pg/mL levels. In addition, there is conflicting data on whether IL-13 levels are similar between (Silvestri et al., Clin. Exp. Allergy 36:1373 [2006], Pukelsheim K, et al., PLoS One 5:e14299 [2010], Doucet J, et al., Dis Markers 35:465-74 [2013]) or elevated (Lee Y C, et al., J Asthma 38:665-71 [2001]; Gauvreau G M, et al., Am J Resp Crit Care 183:1007-14 [2011], Doucet J, et al., Dis Markers 35:465-74 [2013]) in patients with Th2 inflammatory disease relative to healthy controls.

One serum IL-13 assay was described by St. Ledger et al., J. Imm. Methods 350:161-70 (2009) and was initially reported as being highly sensitive. This immunoassay method, which is commercially available under the tradename ERENNA® from Singulex®, uses proprietary monoclonal anti-IL-13 antibodies, paramagnetic microparticles and a specialized instrumentation platform that incorporates a digital counting system that detects single molecules, Id. A subsequent publication, however, indicated that the Erenna® IL-13 Immunoassay was affected by matrix interference and that the extent of matrix interference significantly impacted sensitivity as well as specificity. Fraser S, et al., Bioanalysis 6:1123-9 (2014). That subsequent report provided a revised lower limit of quantitation (LLOQ) of 0.3 pg/mL, Id., an approximately five-fold difference in sensitivity from the initial report. Singulex® has recently made commercially available a new version of the Erenna® IL-13 assay, version 2, which reportedly has a LLOQ of 0.04 pg/mL (Erenna® IL-13 (v2) Immunoassay Kit, Cat. #03-0109-xx Product Information Sheet, available at www(dot)singulex(dot)com). No information is publicly available concerning specificity, however. Moreover, investigators using a customized Singulex® assay system reported a LLOQ of 0.1 pg/mL but were unable to detect IL-13 in 20-60% of asthmatic and healthy control samples tested. Gaye et al., J. Immunol. Methods 426:82-85 (2015). In addition, the product literature for the Simoa™ IL-13 immunoassay reports a LLOQ of 0.0114 pg/mL but provides no information about specificity (Simoa™ IL-13 Immunoassay Product Information Sheet, available at www(dot)quanterix(dot)com). It is recognized in the art that ultrasensitive platforms such as the Erenna® and Simoa™ platforms must be carefully evaluated for specificity. See above and Fisher et al., The AAPS Journal 17:93-101 (2015). Particularly with ultrasensitive platforms, there is a need to ensure that the signal detected is a “true” measurement of an analyte. It is, therefore, important to demonstrate specificity of the signal through a competition or immunodepletion step and illustrate the ability to inhibit a specific signal. Fisher et al., The AAPS Journal 17:93-101 (2015). Accordingly, there remains a need for an IL-13 assay that is both highly sensitive and highly specific.

The invention described herein meets certain of the above-described needs and provides other benefits.

All references cited herein, including patent applications and publications, are incorporated by reference in their entirety for any purpose.

### SUMMARY

The invention provides, at least in part, IL-13 immunoassay methods that are highly sensitive, detecting femtogram/mL levels of IL-13 in greater than 98% of samples tested, and are highly specific, as described herein. Also provided herein are methods of using such highly sensitive and highly specific immunoassay methods to select or identify patients with elevated serum IL-13 levels who are more likely to respond to therapeutic treatments that are Th2 pathway inhibitors (also known as Type 2 pathway inhibitors) as well as to identify asthma patients who are more likely to suffer from severe exacerbations.

Accordingly, in one aspect, high sensitivity and high specificity immunoassay methods for detecting and quantifying IL-13 in samples are provided. In certain embodiments, the samples are biological samples. In certain embodiments, the samples are serum. In certain embodiments, the samples are human serum. In some embodiments, the sensitivity is determined as a lower limit of quantification (LLOQ). In certain embodiments, the LLOQ is between 0.1 fg/mL and 35 fg/mL or between about 0.1 fg/mL and about 35 fg/mL. In certain embodiments, the LLOQ between 1 fg/mL and 30 fg/mL or between about 1 fg/mL and about 30 fg/mL. In certain embodiments, the LLOQ is between 5 fg/mL and 25 fg/mL or between about 5 fg/mL and about 25 fg/mL. In certain embodiments, the LLOQ is between 10 fg/mL and 20 fg/mL or between about 10 fg/mL and about 20 fg/mL. In certain embodiments, the LLOQ is 14 fg/mL or about 14 fg/mL.

In another aspect, sandwich immunoassay methods are provided that comprise a first monoclonal capture antibody that specifically binds IL-13 and a second monoclonal detection antibody that specifically binds IL-13, wherein the first antibody binds a different epitope than the second antibody. In some embodiments, the specificity is determined by an antigen depletion method (also referred to as an immunodepletion method) which comprises incubation of the sample with an excess amount of the first antibody prior to performing the immunoassay method. In certain such embodiments, antigen in the sample is completely depleted thereby producing a signal below the LLOQ in the immunoassay method. In some embodiments, the sample comprises soluble IL-13Rα2 and the soluble IL-13Rα2 does not interfere with the sensitivity or specificity of the immunoassay method.

In yet another aspect, the immunoassay methods comprise a first antibody comprising a variable region comprising a variable heavy chain region comprising HVR-H1 comprising the amino acid sequence of SEQ ID NO: 5, HVR-H2 comprising the amino acid sequence of SEQ ID NO: 6, and HVR-H3 comprising the amino acid sequence of SEQ ID NO: 7 and a variable light chain region comprising HVR-L1 comprising the amino acid sequence of SEQ ID NO: 8, HVR-L2 comprising the amino acid sequence of SEQ ID NO: 9, and HVR-L3 comprising the amino acid sequence of SEQ ID NO: 10. In some embodiments, the first antibody comprises a variable region comprising a variable heavy chain region comprising the amino acid sequence of SEQ ID NO: 1 and a variable light chain region comprising the amino acid sequence of SEQ ID NO: 2. In certain embodiments, the first antibody is an antibody fragment. In certain embodiments, the first antibody is an antibody fragment which is F(ab′)₂ or Fab. In certain embodiments, the first antibody is an antibody fragment which is Fab, F(ab′)₂, Fab′, or Fv. In some embodiments, the immunoassay methods comprise a second antibody comprising a variable region comprising a variable heavy chain region comprising HVR-H1 comprising the amino acid sequence of SEQ ID NO: 13, HVR-H2 comprising the amino acid sequence of SEQ ID NO: 14, and HVR-H3 comprising the amino acid sequence of SEQ ID NO: 15 and a variable light chain region comprising HVR-L1 comprising the amino acid sequence of SEQ ID NO: 16, HVR-L2 comprising the amino acid sequence of SEQ ID NO: 17, and HVR-L3 comprising the amino acid sequence of SEQ ID NO: 18. In some embodiments, the second antibody comprises a variable region comprising a variable heavy chain region comprising the amino acid sequence of SEQ ID NO: 12 and a variable light chain region comprising the amino acid sequence of SEQ ID NO: 11.

In yet still another aspect, the immunoassay methods further comprise a third antibody, wherein the third antibody specifically binds to the second antibody and is detectably labeled. In some embodiments, the second antibody is labeled with a hapten and the third antibody is an anti-hapten antibody. In some embodiments, the hapten is digoxigenen and the anti-hapten antibody is an anti-digoxigenin monoclonal antibody conjugated with fluorescent latex.

The methods of treatment and diagnosis as provided herein can be applied to patients suffering from asthma, eosinophilic disorder, respiratory disorders, IL-13 mediated disorder, Th2-associated disorder, and/or IgE-mediated disorder, or symptoms related to those disorders. Patients suffering from asthma-like symptoms, include patients that have not been diagnosed with asthma may be treated according to the methods provided herein.

According to one embodiment, a patient treated according to the methods provided herein suffers from asthma, an eosinophilic disorder, a respiratory disorder, an IL-13 mediated disorder, a Th2-associated disorder (Type-2 associated disorder) and/or an IgE-mediated disorder, or symptoms related to those disorders. According to another embodiment, the patient treated according to the methods provided herein is suffering from asthma, eosinophilic disorder, respiratory disorders, IL-13 mediated disorder, Th2-associated disorder and/or IgE-mediated disorder, or symptoms related to those disorders, and is 2 years old or older, 12 years old or older, 18 years old or older, 19 years old or older, between 2 and 18 years old, between 2 and 17 years old, between 12-17 years old, between 12 and 18 years old, between 2 and 75 years old, between 12 and 75 years old, or between 18 and 75 years old.

In some embodiments, methods of identifying an asthma patient or a Th2-associated disease (Type 2-associated disease) patient who is likely to be responsive to treatment with a Th2 pathway inhibitor are provided. In some embodiments, the method comprises determining whether the patient has elevated levels of IL-13 using any of the IL-13 immunoassay methods described in the Summary above compared to a reference level, wherein elevated IL-13 indicates that the patient is likely to be responsive to treatment with the Th2 pathway inhibitor.

In some embodiments, methods of identifying an asthma patient or a respiratory disorder patient who is likely to suffer from severe exacerbations are provided. In some embodiments, the method comprises determining whether the patient has elevated levels of IL-13 using any of the IL-13 immunoassay methods described in the Summary above compared to a reference level, wherein elevated IL-13 indicates that the patient is likely to suffer from an increase in severe exacerbations. In some embodiments, the methods comprise obtaining a biological sample from the patient, measuring the IL-13 level, comparing the IL-13 level detected in the sample to a reference level, and predicting that the patient is likely to suffer from severe exacerbations when the IL-13 level measured in the sample is elevated compared to the reference level. In some embodiments, the methods comprise (a) measuring the IL-13 in a biological sample from the patient; (b) comparing the IL-13 level measured in (a) to a reference level; and (c) identifying the patient as more likely to suffer from severe exacerbations when the IL-13 level measured in (a) is above the reference level. In some embodiments, the reference level is the median level of IL-13 in a reference population.

In some embodiments, methods of monitoring an asthma patient or a Th2-associated disease (Type 2-associated disease) patient being treated with a Th2 Pathway inhibitor (Type 2 pathway inhibitor) are provided. In some embodiments, the method comprises determining whether the patient has elevated levels of IL-13 using any of the IL-13 immunoassay methods described in the Summary above. In some embodiments, the method further comprises determining a treatment regimen for the Th2 pathway inhibitor. In some such embodiments, the determination of IL-13 level indicates continuing therapy with the Th2 pathway inhibitor or discontinuing therapy with the Th2 pathway inhibitor.

In any of the embodiments described herein, the methods may comprise the steps of a) determining the level of IL-13 in a sample obtained from the patient using any of the IL-13 immunoassay methods described in the Summary above; and b) comparing the levels of IL-13 determined in step a) to a reference level. In some embodiments, the methods further comprise c) stratifying said patient into the category of responder or non-responder based on the comparison obtained in step b). In some embodiments, a method further comprises selecting a therapy comprising a Th2 pathway inhibitor if the patient is a responder.

In some embodiments, methods of predicting the response of a patient suffering from asthma or a Th2-associated disease (Type 2-associated disease) to a therapy comprising a Th2 pathway inhibitor (Type 2 pathway inhibitor) are provided. In some embodiments, the method comprises obtaining a biological sample from the patient and measuring the IL-13 level in the sample using any of the IL-13 immunoassay methods described in the Summary above. In some embodiments, the method comprises comparing the IL-13 level detected in the sample to a reference level. In some embodiments, the method comprises predicting that the patient will respond to the therapy when the IL-13 level measured in the sample is elevated compared to the reference level and predicting that the patient will not respond to the therapy when the IL-13 level measured in the sample is reduced compared to the reference level.

In some embodiments, methods of predicting responsiveness of an asthma patient or a Th2-associated disease patient (Type 2-associated disease) to a Th2 pathway inhibitor (Type 2 pathway inhibitor) treatment are provided. In some embodiments, the method comprises measuring the IL-13 level in a biological sample from the patient using any of the IL-13 immunoassay methods described in the Summary above. In some embodiments, an elevated IL-13 level compared to a reference level identifies the patient as one who is likely to respond to the Th2 pathway inhibitor treatment.

In some embodiments, methods of identifying a patient suffering from asthma or a Th2-associated disease (Type 2-associated disease) as likely to respond to a therapy comprising a Th2 pathway inhibitor (Type 2 pathway inhibitor) are provided. In some embodiments, the method comprises measuring the IL-13 level in a biological sample from the patient using any of the IL-13 immunoassay methods described in the Summary above. In some embodiments, the method further comprises comparing the measured IL-13 level to a reference level. In some embodiments, the method comprises identifying the patient as more likely to respond to the therapy comprising the Th2 pathway inhibitor when the measured IL-13 level is above the reference level.

In some embodiments, methods of treating patients having asthma or a Th2-associated disease (Type 2-associated disease) are provided. In some embodiments, the method comprises measuring the IL-13 level in a biological sample from the patient using any of the IL-13 immunoassay methods described in the Summary above. In some embodiments, the method comprises comparing the measured IL-13 level to a reference level. In some embodiments, the method comprises identifying the patient as more likely to respond a therapy comprising a Th2 pathway inhibitor when the measured IL-13 level is above the reference level. In some embodiments, the method comprises administering the therapy when the measured IL-13 level is above the reference level, thereby treating the asthma or Th2-associated disease.

In some embodiments, a method of treating asthma or a Th2-associated disease (Type 2-associated disease) in a patient comprises administering to the patient a therapeutically effective amount of a Th2 pathway inhibitor (Type 2 pathway inhibitor), wherein a biological sample obtained from the patient has been determined to have elevated IL-13 levels using any of the IL-13 immunoassay methods described in the Summary above.

In some embodiments, a method of treating asthma or a Th2-associated disease (Type 2-associated disease) in a patient comprises administering to the patient a therapeutically effective amount of a Th2 pathway inhibitor (Type 2 pathway inhibitor), wherein the patient has been selected for treatment based on elevated IL-13 levels in biological sample obtained from the patient using any of the IL-13 immunoassay methods described in the Summary above.

In any of the embodiments described herein, the reference level may be the median, mean, or average level of IL-13 in a reference population. In any of the embodiments described herein, the reference level may be the median level of IL-13 in a reference population. In any of the embodiments described herein, the reference level may be the mean level of IL-13 in a reference population. In any of the embodiments described herein, the reference level may be the average level of IL-13 in a reference population. Nonlimiting exemplary reference populations include patients with asthma, patients with moderate to severe asthma, patients with idiopathic pulmonary fibrosis, patients with atopic dermatitis, healthy individuals, and a group including healthy individuals and any of the aforementioned patients. In some embodiments, a reference population comprises patients with moderate to severe asthma. Further nonlimiting exemplary reference populations include patients with a Th2-associated disease such as asthma, atopic dermatitis, idiopathic pulmonary fibrosis, allergic rhinitis, fibrosis, inflammatory bowel disease, ulcerative colitis, Crohn's disease, chronic obstructive pulmonary disease, and hepatic fibrosis.

In some embodiments, if the level of IL-13 is above the reference level, the patient is stratified into the category of responder.

In some embodiments, the biological sample is selected from blood, serum, plasma. In some embodiments, the biological sample is serum. In some embodiments, the biological sample is plasma. In some embodiments, the biological sample is obtained from an asthma patient. In certain embodiments, the patient according to the methods described above is suffering from moderate to severe asthma. In certain embodiments, the asthma or respiratory disorder is uncontrolled on a corticosteroid. In certain embodiments, the corticosteroid is an inhaled corticosteroid. In certain embodiments, the inhaled corticosteroid is Qvar®, Pulmicort®, Symbicort®, Aerobid®, Flovent®, Flonase®, Advair® or Azmacort®. In one embodiment, the patient is also being treated with a second controller. In certain embodiments, the second controller is a long acting bronchial dilator (LABD). In certain embodiments, the LABD is a long-acting beta-2 agonist (LABA), leukotriene receptor antagonist (LTRA), long-acting muscarinic antagonist (LAMA), theophylline, or oral corticosteroids (OCS). In certain embodiments, the LABD is Symbicort®, Advair®, Brovana®, Foradil®, Perforomist™ or Serevent®.

In any of the embodiments described herein, the patient may be 0-17 years old, 2-17 years old, 2-6 years old, 6-11 years old, 8-17 years old, 12-17 years old, 2 years old or older, 6 years old or older, or 12 years old or older. In some embodiments, the patient is 18 years or older. In any of the embodiments described herein, the patient may be a human.

In any of the embodiments described herein, the Th2 pathway inhibitor may inhibit the target ITK, BTK, IL-9 (e.g., MEDI-528), IL-5 (e.g., Mepolizumab, CAS No. 196078-29-2; resilizumab), IL-13 (e.g., IMA-026, IMA-638 (also referred to as, anrukinzumab, INN No. 910649-32-0; QAX-576; IL4/IL13 trap), tralokinumab (also referred to as CAT-354, CAS No. 1044515-88-9); AER-001, ABT-308 (also referred to as humanized 13C5.5 antibody), IL-4 (e.g., AER-001, IL4/IL13 trap), IL-17, OX40L, TSLP, IL-25, IL-33 and IgE (e.g., XOLAIR, QGE-031; MEDI-4212; quilizumab); and receptors such as: IL-9 receptor, IL-5 receptor (e.g., MEDI-563 (benralizumab, CAS No. 1044511-01-4), IL-4receptor alpha (e.g., AMG-317, AIR-645, dupilumab), IL-13receptoralpha1 (e.g., R-1671) and IL-13receptoralpha2, OX40, TSLP-R, IL-7Ralpha (a co-receptor for TSLP), IL17RB (receptor for IL-25), ST2 (receptor for IL-33), CCR3, CCR4, CRTH2 (e.g., AMG-853, AP768, AP-761, MLN6095, ACT129968), FcepsilonRI, FcepsilonRII/CD23 (receptors for IgE), Flap (e.g., GSK2190915), Syk kinase (R-343, PF3526299); CCR4 (AMG-761), TLR9 (QAX-935), or is a multi-cytokine inhibitor of CCR3, IL5, IL3, GM-CSF (e.g., TPI ASM8).

In any of the embodiments described herein, the Th2 pathway inhibitor (Type 2 pathway inhibitor) is an IL-13 inhibitor, an agent that inhibits both IL-13 and IL-4, an agent that inhibits both IL-13 and IL-17, or an anti IgE binding agent. In any of the embodiments described herein, the Th2 pathway inhibitor is an anti-IL-13 antibody. In certain embodiments, the anti-IL-13 antibody is an antibody comprising a VH comprising a sequence selected from SEQ ID NOs: 1, 3, and 24, and a VL comprising a sequence selected from SEQ ID NO: 2, 4, and 25; an anti-IL13 antibody comprising HVRH1, HVRH2, HVRH3, HVRL1, HVRL2, and HVRL3, wherein the respective HVRs have the amino acid sequence of SEQ ID NO.: 5, SEQ ID NO.: 6, SEQ ID NO.: 7, SEQ ID NO.: 8, SEQ ID NO.: 9, and SEQ ID NO.: 10; or lebrikizumab.

In some embodiments, the patient is administered a flat dose of 37.5 mg, or 125 mg or 250 mg anti-IL-13 antibody or lebrikizumab every four weeks. In some embodiments, the anti-IL-13 antibody is administered subcutaneously. In some embodiments, the anti-IL-13 antibody is administered using a prefilled syringe or autoinjector device.

In certain embodiments, the anti-IL-13 antibody is a bispecific antibody. In certain embodiments, the anti-IL-13 antibody is a bispecific antibody that also binds IL-4. In certain embodiments, the anti-IL-13 antibody is a bispecific antibody that also binds IL-17. In some embodiments, the anti-IL-13 bispecific antibody comprises an anti-IL-13 VH/VL unit comprising a VH comprising a sequence selected from SEQ ID NOs: 1, 3, and 24, and a VL comprising a sequence selected from SEQ ID NO: 2, 4, and 25; or an anti-IL13 VH/VL unit comprising HVRH1, HVRH2, HVRH3, HVRL1, HVRL2, and HVRL3, wherein the respective HVRs have the amino acid sequence of SEQ ID NO.: 5, SEQ ID NO.: 6, SEQ ID NO.: 7, SEQ ID NO.: 8, SEQ ID NO.: 9, and SEQ ID NO.: 10.

In any of the embodiments described herein, the Th2 pathway inhibitor (Type 2 pathway inhibitor) is an anti-IL-13/anti-IL-17 bispecific antibody. In some embodiments, the anti-IL-13/anti-IL-17 bispecific antibody comprises an anti-IL-13 VH/VL unit comprising HVRH1, HVRH2, HVRH3, HVRL1, HVRL2, and HVRL3, wherein the respective HVRs have the amino acid sequence of SEQ ID NO.: 5, SEQ ID NO.: 6, SEQ ID NO.: 7, SEQ ID NO.: 8, SEQ ID NO.: 9, and SEQ ID NO.: 10; and an anti-IL-17 VH/VL unit comprising HVRH1, HVRH2, HVRH3, HVRL1, HVRL2, and HVRL3, wherein the respective HVRs have the amino acid sequence of SEQ ID NO.: 26, SEQ ID NO.: 27, SEQ ID NO.: 28, SEQ ID NO.: 29, SEQ ID NO.: 30, and SEQ ID NO.: 31. In some embodiments, anti-IL-13/anti-IL-17 bispecific antibody comprises an anti-IL-13 VH/VL unit comprising a VH comprising an amino acid sequence selected from SEQ ID NOs: 1, 3, and 24, and a VL comprising an amino acid sequence selected from SEQ ID NO: 2, 4, and 25; and an anti-IL-17 VH/VL unit comprising a VH comprising the amino acid sequence of SEQ ID NO: 32 and a VL comprising the amino acid sequence of SEQ ID NO: 33.

In any of the embodiments described herein, the Th2 pathway inhibitor may be an anti-IgE antibody. In certain embodiments, the anti-IgE antibody is (i) the XOLAIR® antibody or (ii) an anti-IgE antibody comprising a variable heavy chain region and a variable light chain region, wherein the variable heavy chain region is SEQ ID NO:22 and the variable light chain region is SEQ ID NO:23.

In one embodiment, a patient treated with a Th2 pathway inhibitor (Type 2 pathway inhibitor) according to this invention is also treated with one, two, three or more therapeutic agents. In one embodiment, the patient is an asthma patient. According to one embodiment, the patient is treated with the Th2 pathway inhibitor and one, two, three or more therapeutic agents, wherein at least one therapeutic agent, other than the Th2 inhibitor, is a corticosteroid, a leukotriene antagonist, a LABA, a corticosteroid/LABA combination composition, a theophylline, cromolyn sodium, nedocromil sodium, omalizumab, a LAMA, a MABA, a 5-Lipoxygenase Activating Protein (FLAP) inhibitor, or an enzyme PDE-4 inhibitor. According to one aspect of the invention, a Th2 pathway inhibitor is administered to an asthma patient diagnosed as having elevated IL-13, wherein the diagnosis comprises the use any of the IL-13 immunoassay methods described in the Summary above. In one further embodiment, the asthma patient is uncontrolled on a corticosteroid prior to the treatment. In another embodiment, the asthma patient is also being treated with a second controller. In one embodiment, the second controller is a corticosteroid, a LABA or a leukotriene antagonist. In a further embodiment, the asthma patient is suffering from moderate to severe asthma. Thus, in one embodiment, the patient to be treated with the Th2 pathway inhibitor is a moderate to severe asthma patient who is uncontrolled on a corticosteroid prior to treatment with the Th2 pathway inhibitor, and then is treated with the Th2 pathway inhibitor and one, two, three or more controllers. In one embodiment, at least one of the controllers is a corticosteroid. In a further embodiment, such patient is treated with a Th2 pathway inhibitor, a corticosteroid and another controller. In another embodiment, the patient is suffering from mild asthma but is not being treated with a corticosteroid. It should be understood that the therapeutic agents may have different treatment cycles as compared with the Th2 inhibitor and, consequently can be administered at different times compared to the Th2 inhibitor as a part of the patient's treatment. Therefore, according to one embodiment, a method of treatment according to this invention comprises the steps of administering to a patient a Th2 pathway inhibitor and optionally, administering at least one, two or three additional therapeutic agents. In one embodiment, the Th2 pathway inhibitor is present in a composition with another therapeutic agent. In another embodiment, the Th2 pathway inhibitor is not present in a composition with another therapeutic agent.

According to another embodiment, the invention comprises a method for treating asthma comprising administering an anti-IL-13 antibody comprising a VH comprising a sequence selected from SEQ ID NOs: 1, 3, and 24, and a VL comprising a sequence selected from SEQ ID NO: 2, 4, and 25; an anti-IL13 antibody comprising HVRH1, HVRH2, HVRH3, HVRL1, HVRL2, and HVRL3, wherein the respective HVRs have the amino acid sequence of SEQ ID NO.: 5, SEQ ID NO.: 6, SEQ ID NO.: 7, SEQ ID NO.: 8, SEQ ID NO.: 9, and SEQ ID NO.: 10; or lebrikizumab; as a flat dose. In one embodiment, the anti-IL-13 antibody is administered as a flat dose (i.e., not weight dependent) of 37.5 mg, or a flat dose of 125 mg, or a flat dose of 250 mg, by subcutaneous injection once every 4 weeks. In some embodiments, the patient is diagnosed as having elevated IL-13 using any of the IL-13 immunoassay methods described in the Summary above. In some embodiments, the patient is additionally diagnosed as having elevated levels of one or more Th2-associated biomarkers selected from periostin, FeNO, eosinophils, and IgE. In some embodiments, the patient is diagnosed as having elevated IL-13 using any of the IL-13 immunassay methods described in the Summary above and elevated blood eosinophil levels. In some embodiments, the blood eosinophil levels are determined as 300 cells/microliter or above. In some embodiments, the patient is diagnosed as having elevated IL-13 using any of the IL-13 immunassay methods described in the Summary above, elevated serum periostin and elevated blood eosinophil levels. In some embodiments, blood eosinophil levels are determined as 300 cells/microliter or above.

According to another embodiment, an anti-IL-13 antibody comprising a VH comprising a sequence selected from SEQ ID NOs: 1, 3, and 24, and a VL comprising a sequence selected from SEQ ID NO: 2, 4, and 25; an anti-IL13 antibody comprising HVRH1, HVRH2, HVRH3, HVRL1, HVRL2, and HVRL3, wherein the respective HVRs have the amino acid sequence of SEQ ID NO.: 5, SEQ ID NO.: 6, SEQ ID NO.: 7, SEQ ID NO.: 8, SEQ ID NO.: 9, and SEQ ID NO.: 10; or lebrikizumab is administered to treat asthma in a therapeutically effective amount sufficient to reduce the rate of exacerbations of the patient over time or improve FEV₁. In yet another embodiment, the invention comprises a method for treating asthma comprising administering an anti-IL-13 antibody comprising a VH comprising a sequence selected from SEQ ID NOs: 1, 3, and 24, and a VL comprising a sequence selected from SEQ ID NO: 2, 4, and 25; an anti-IL13 antibody comprising HVRH1, HVRH2, HVRH3, HVRL1, HVRL2, and HVRL3, wherein the respective HVRs have the amino acid sequence of SEQ ID NO.: 5, SEQ ID NO.: 6, SEQ ID NO.: 7, SEQ ID NO.: 8, SEQ ID NO.: 9, and SEQ ID NO.: 10; or lebrikizumab as a flat dose (i.e., not weight dependent) of 37.5 mg, or a flat dose of 125 mg, or a flat dose of 250 mg. In certain embodiments, the dose is administered by subcutaneous injection once every 4 weeks for a period of time. In certain embodiments, the period of time is 6 months, one year, two years, five years, ten years, 15 years, 20 years, or the lifetime of the patient. In certain embodiments, the asthma is severe asthma and the patient is inadequately controlled or uncontrolled on inhaled corticosteroids plus a second controller medication. In some embodiments, the patient is diagnosed as having elevated IL-13 using any of the IL-13 immunoassay methods described in the Summary above and the patient is selected for treatment with an anti-IL13 antibody as described above. In another embodiment, the method comprises treating an asthma patient with an anti-IL13 antibody as described above where the patient was previously diagnosed with having elevated IL-13 using any of the IL-13 immunoassay methods described in the Summary above. In some embodiments, the patient was additionally previously diagnosed as having elevated levels of one or more Th2-associated biomarkers selected from periostin, FeNO, eosinophils, and IgE. In some embodiments, the patient was previously diagnosed as having elevated IL-13 using any of the IL-13 immunassay methods described in the Summary above and elevated blood eosinophil levels. In some embodiments, the blood eosinophil levels were determined as 300 cells/microliter or above. In some embodiments, the patient was previously diagnosed as having elevated IL-13 using any of the IL-13 immunassay methods described in the Summary above, elevated serum periostin and elevated blood eosinophil levels. In some embodiments, blood eosinophil levels were determined as 300 cells/microliter or above.

The present invention provides a therapeutic agent that is a Th2 pathway inhibitor (Type 2 pathway inhibitor) for use in treating asthma or a Th2-associated disease (Type 2-associated disease) in a patient, wherein the patient has elevated IL-13 levels determined by using any of the IL-13 immunoassay methods described in the Summary above. In some embodiments, the target for inhibition in the Th2 pathway is selected from: IL-9, IL-5, IL-13, IL-4, IL-17, OX40L, TSLP, IL-25, IL-33 and IgE; and receptors such as: IL-9 receptor, IL-5 receptor, IL-4receptor alpha, IL-13receptoralpha1 and IL-13receptoralpha2, OX40, TSLP-R, IL-7Ralpha (a co-receptor for TSLP), IL17RB (receptor for IL-25), ST2 (receptor for IL-33), CCR3, CCR4, CRTH2, FcepsilonRI and FcepsilonRII/CD23 (receptors for IgE). In one embodiment, the patient to be treated according to the methods of the present invention is suffering from mild to severe asthma, optionally moderate to severe asthma, and whose asthma is uncontrolled on a corticosteroid.

In another aspect, uses of a kit for measuring the level of IL-13 in a sample obtained from an asthma patient for stratifying/classifying asthma patients into likely responders and non-responders for therapeutic treatment with a Th2 pathway inhibitor. In certain embodiments, the use comprises the steps of: (a) determining the level of IL-13 in a sample obtained from an asthma patient using any of the IL-13 immunoassay methods described in the Summary above; (b) comparing the level of IL-13 determined in step (a) to a reference level; and (c) stratifying said patient into the category of responder or non-responder based on the comparison obtained in step (b).

In certain embodiments, the Th2 pathway inhibitor (Type 2 pathway inhibitor) according to the uses above inhibits the target ITK, BTK, IL-9 (e.g., MEDI-528), IL-5 (e.g., Mepolizumab, CAS No. 196078-29-2; resilizumab), IL-13 (e.g., IMA-026, IMA-638 (also referred to as, anrukinzumab, INN No. 910649-32-0; QAX-576; IL4/IL13 trap), tralokinumab (also referred to as CAT-354, CAS No. 1044515-88-9); AER-001, ABT-308 (also referred to as humanized 13C5.5 antibody), IL-4 (e.g., AER-001, IL4/IL13 trap), IL-17, OX40L, TSLP, IL-25, IL-33 and IgE (e.g., XOLAIR, QGE-031; MEDI-4212; quilizumab); and receptors such as: IL-9 receptor, IL-5 receptor (e.g., MEDI-563 (benralizumab, CAS No. 1044511-01-4), IL-4receptor alpha (e.g., AMG-317, AIR-645, dupilumab), IL-13receptoralpha1 (e.g., R-1671) and IL-13receptoralpha2, OX40, TSLP-R, IL-7Ralpha (a co-receptor for TSLP), IL17RB (receptor for IL-25), ST2 (receptor for IL-33), CCR3, CCR4, CRTH2 (e.g., AMG-853, AP768, AP-761, MLN6095, ACT129968), FcepsilonRI, FcepsilonRII/CD23 (receptors for IgE), Flap (e.g., GSK2190915), Syk kinase (R-343, PF3526299); CCR4 (AMG-761), TLR9 (QAX-935), or is a multi-cytokine inhibitor of CCR3, IL5, IL3, GM-CSF (e.g., TPI ASM8).

In yet another aspect, kits for measuring the level of IL-13 in a biological sample obtained from an asthma patient or a patient suffering from a Th2-associated disease (Type 2-associated disease) are provided. In some embodiments, the kit comprises instructions for (i) measuring the IL-13 level using any of the IL-13 immunoassay methods described in the Summary above, (ii) comparing the level of IL-13 to a reference level, and (iii) stratifying said patient into the category of responder or non-responder based on the comparison. In some embodiments, the kit comprises at least one, at least two, or at least three antibodies. In some embodiments, the kit comprises a first monoclonal capture antibody that specifically binds IL-13 and a second monoclonal detection antibody that specifically binds IL-13, wherein the first antibody binds a different epitope than the second antibody. In some embodiments, the kit comprises a first antibody comprising a variable region comprising a variable heavy chain region comprising HVR-H1 comprising the amino acid sequence of SEQ ID NO: 5, HVR-H2 comprising the amino acid sequence of SEQ ID NO: 6, and HVR-H3 comprising the amino acid sequence of SEQ ID NO: 7 and a variable light chain region comprising HVR-L1 comprising the amino acid sequence of SEQ ID NO: 8, HVR-L2 comprising the amino acid sequence of SEQ ID NO: 9, and HVR-L3 comprising the amino acid sequence of SEQ ID NO: 10. In some embodiments, the first antibody comprises a variable region comprising a variable heavy chain region comprising the amino acid sequence of SEQ ID NO: 1 and a variable light chain region comprising the amino acid sequence of SEQ ID NO: 2. In certain embodiments, the first antibody is an antibody fragment. In certain embodiments, the first antibody is an antibody fragment which is F(ab′)₂ or Fab. In certain embodiments, the first antibody is antibody fragment which is Fab, F(ab′)₂, Fab′, or Fv. In some embodiments, the immunoassay methods comprise a second antibody comprising a variable region comprising a variable heavy chain region comprising HVR-H1 comprising the amino acid sequence of SEQ ID NO: 13, HVR-H2 comprising the amino acid sequence of SEQ ID NO: 14, and HVR-H3 comprising the amino acid sequence of SEQ ID NO: 15 and a variable light chain region comprising HVR-L1 comprising the amino acid sequence of SEQ ID NO: 16, HVR-L2 comprising the amino acid sequence of SEQ ID NO: 17, and HVR-L3 comprising the amino acid sequence of SEQ ID NO: 18. In some embodiments, the second antibody comprises a variable region comprising a variable heavy chain region comprising the amino acid sequence of SEQ ID NO: 12 and a variable light chain region comprising the amino acid sequence of SEQ ID NO: 11. In some embodiments, the kit comprises a third antibody, wherein the third antibody specifically binds to the second antibody and is detectably labeled. In some embodiments, the second antibody is labeled with a hapten and the third antibody is an anti-hapten antibody. In some embodiments, the hapten is digoxigenen and the anti-hapten antibody is an anti-digoxigenin monoclonal antibody conjugated with fluorescent latex. In certain embodiments, the kit comprises a package insert containing information describing the uses provided above.

In still yet another aspect, kits for diagnosing an asthma subtype in a patient are provided, the kits comprising: (1) determining the level of IL-13 in a serum sample obtained from the patient using any of the IL-13 immunoassay methods described in the Summary above; and (2) instructions for measuring the level of IL-13 in the serum sample, wherein the elevated expression level of IL-13 is indicative of the asthma subtype.

In some embodiments, the kit further comprises a package insert for determining whether an asthma patient or Th2-associated disease (Type 2-associated disease) patient has elevated IL-13 levels or not. In some embodiments, the kit further comprises a package insert for determining whether an asthma patient or Th2-associated disease patient is likely to respond to a Th2 pathway inhibitor. In some embodiments, the kit further comprises a package insert containing information describing any of the uses provided above. In some embodiments, the kit further comprises an empty container to hold a biological sample. In some embodiments, the kit comprises reagents for determining the levels of IL-13.

In still another aspect, methods of treating of a patient suffering from asthma or a Th2-associated disease (Type 2-associated disease) comprising administering a Th2 pathway inhibitor (Type 2 pathway inhibitor) to the patient diagnosed as having elevated circulating IL-13 levels are provided. In certain embodiments, the methods comprise the step of diagnosing the patient as having elevated IL-13 levels using any of the IL-13 immunoassay methods described in the Summary above. In certain embodiments, the methods further comprise the step of retreating the patient with the Th2 pathway inhibitor if the patient is determined to have elevated circulating IL-13 levels. In certain embodiments, serum or plasma from the patient is used to determine whether the patient has elevated circulating IL-13 levels.

In any of the embodiments described herein, the levels of one or more Th2-associated biomarkers (Type 2 associated biomarkers) is determined in addition to the IL-13 level. In some embodiments, the additional Th2-associated biomarker is periostin. In some embodiments, the additional Th2-associated biomarker is serum periostin. In some embodiments, the additional Th2-associated biomarker is FeNO. In some embodiments, the additional Th2-associated biomarker is eosinophils. In some embodiments, the additional Th2-associated biomarker is blood eosinophils. In some embodiments, the additional Th2-associated biomarker is IgE.

### BRIEF DESCRIPTION OF THE DRAWINGS

FIG. 1. Assessment of the sensitivity and specificity of the Erenna® IL-13 assay as described in Example 2. Serum samples from asthmatic patients (n=10) were measured with (right side) and without (left side) pre-incubation with excess assay capture antibody. The higher IL-13 values (n=4) remained above LLOQ with pre-incubation with excess assay capture antibody. The dotted line indicates the LLOQ recommended by the manufacturer, 0.39 pg/mL.

FIGS. 2A and 2B. Assay modifications improved the specificity, but not the sensitivity, of the Erenna® IL-13 Immunoassay, as described in Example 2. FIG. 2A shows three healthy volunteer (HV) serum samples that were measured following the manufacturer's standard protocol (FIG. 2A, left side, HV) and after pre-incubation with excess capture antibody-coated microparticles (FIG. 2A, right side, HV+Capture Ab). FIG. 2B shows the same three HV samples measured following dilution 1:1 (V/V) with high salt buffer (FIG. 2B, left side, HV) and after pre-incubation with excess capture antibody-coated microparticles (FIG. 2B, right side, HV+Capture Ab). The dashed line in each of FIG. 2A and FIG. 2B represents the LLOQ. Note that the LLOQ in FIG. 2B was modified compared to the LLOQ in FIG. 2A to account for the 1:1 (V/V) sample dilution with high salt buffer.

FIG. 3. Modified assay conditions improved the specificity of the Erenna® IL-13 Immunoassay but diminished the ability to detect native IL-13 in human serum samples as described in Example 2. Serum samples from HV (n=10), asthma patients (n=10) and IPF patients (n=10) were measured without and with excess capture antibody-coated microparticles pre-incubation. The dotted line indicates the modified Erenna® IL-13 Immunoassay LLOQ of 0.78 pg/mL.

FIG. 4. Specificity of IMACT IL-13 assay as described in Example 3. Serum from healthy volunteers and patients with different Th2-associated diseases (n=101 in total) were measured with (right side, Serum+Capture Ab) and without (left side, Serum) pre-incubation with excess assay first capture antibody. The dotted line indicates the IMPACT IL-13 assay LLOQ of 0.014 pg/mL.

FIG. 5. Serum IL-13 levels by IMPACT IL-13 assay as described in Example 3. Individual values for samples from HV (n=50), asthma patients (n=34), IPF patients (n=32), and atopic dermatits patients (n=25) are indicated as well as the median for each group. The dotted line indicates the IMPACT IL-13 assay LLOQ of 0.014 pg/mL. Mann-Whitney test was performed to compare the means between HV and asthma, IPF or atopic dermatitis, respectively. ** indicate the P value for each comparison was <0.0001.

FIG. 6. Correlation of baseline (week 0) serum IL-13 levels with blood eosinophils counts, serum periostin, FeNO and serum IgE levels as described in Example 4. Spearman rank-order correlation coefficient (p) for each comparison is indicated in the respective scatter plot.

FIGS. 7A and 7B. Mean percentage change at Week 12 compared to baseline in FEV₁ by IL-13 status as described in Example 4. FIG. 7A shows the results for placebo and each of the three dose groups (37.5 mg lebrikizumab every 4 weeks, 125 mg lebrikizumab every 4 weeks, or 250 mg lebrikizumab every 4 weeks) in the serum IL-13 high group, those subjects with serum IL-13 at or above the median at baseline; FIG. 7B shows the results for placebo and each of the three dose groups (37.5 mg lebrikizumab every 4 weeks, 125 mg lebrikizumab every 4 weeks, or 250 mg lebrikizumab every 4 weeks) in the serum IL-13 low group, those subjects with serum IL-13 below the median at baseline.

FIG. 8. Asthma exacerbation rate over the placebo-controlled period in the serum IL-13 high group (left 4 bars) and in the serum IL-13 low group (right 4 bars) as described in Example 4. The gray arrow indicates the observed exacerbation rate reduction, percentage (95% CI), for lebrikizumab (LEB) for each of the three dose groups (37.5 mg lebrikizumab every 4 weeks, 125 mg lebrikizumab every 4 weeks, or 250 mg lebrikizumab every 4 weeks) versus placebo.

### DETAILED DESCRIPTION

All references cited herein, including patent applications and publications, are incorporated by reference in their entirety for any purpose.

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

### Certain Definitions

For purposes of interpreting this specification, the following definitions will apply and whenever appropriate, terms used in the singular will also include the plural and vice versa. In the event that any definition set forth below conflicts with any document incorporated herein by reference, the definition set forth below shall control.

As used in this specification and the appended claims, the singular forms “a,” “an” and “the” include plural referents unless the context clearly dictates otherwise. Thus, for example, reference to “a protein” or an “antibody” includes a plurality of proteins or antibodies, respectively; reference to “a cell” includes mixtures of cells, and the like.

Ranges provided in the specification and appended claims include both end points and all points between the end points. Thus, for example, a range of 2.0 to 3.0 includes 2.0, 3.0, and all points between 2.0 and 3.0

The term “detecting” is used herein in the broadest sense to include both qualitative and quantitative measurements of a target molecule. Detecting includes identifying the mere presence of the target molecule in a sample as well as determining whether the target molecule is present in the sample at detectable levels.

A “capture antibody” refers to an antibody that specifically binds a target molecule in a sample. Under certain conditions, the capture antibody forms a complex with the target molecule such that the antibody-target molecule complex can be separated from the rest of the sample. In certain embodiments, such separation may include washing away substances or material in the sample that did not bind the capture antibody. In certain embodiments, a capture antibody may be attached to a solid support surface, such as, for example but not limited to, a plate or a bead.

A “detection antibody” refers to an antibody that specifically binds a target molecule in a sample or in a sample-capture antibody combination material. Under certain conditions, the detection antibody forms a complex with the target molecule or with a target molecule-capture antibody complex. A detection antibody is capable of being detected either directly through a label, which may be amplified, or indirectly, e.g., through use of another antibody that is labeled (e.g., detectably labeled) and that binds the detection antibody. For direct labeling, the detection antibody is typically conjugated to a moiety that is detectable by some means, for example, including but not limited to, biotin or ruthenium.

The terms “label” or “detectable label” refers to any chemical group or moiety that can be linked to a substance that is to be detected or quantitated, e.g., an antibody. Typically, a label is a detectable label that is suitable for the sensitive detection or quantification of a substance. Examples of detectable labels include, but are not limited to, luminescent labels, e.g., fluorescent, phosphorescent, chemiluminescent, bioluminescent and electrochemiluminescent labels, radioactive labels, enzymes, particles, magnetic substances, electroactive species and the like. Alternatively, a detectable label may signal its presence by participating in specific binding reactions. Examples of such labels include haptens, antibodies, biotin, streptavidin, his-tag, nitrilotriacetic acid, glutathione S-transferase, glutathione and the like.

The term “detection means” refers to a moiety or technique used to detect the presence of the detectable antibody through signal reporting that is then read out in an assay. Typically, detection means employ reagents that amplify an immobilized label such as the label captured onto a microtiter plate, e.g., avidin or streptavidin-HRP.

“Photoluminescence” refers to a process whereby a material luminesces subsequent to the absorption by that material of light (alternatively termed electromagnetic radiation). Fluorescence and phosphorescence are two different types of photoluminescence. “Chemiluminescent” processes involve the creation of the luminescent species by a chemical reaction. “Electro-chemiluminescence” or “ECL” is a process whereby a species, e.g., an antibody, luminesces upon the exposure of that species to electrochemical energy in an appropriate surrounding chemical environment.

The term “sensitivity” refers to the ability of an assay to detect an analyte. In one embodiment, sensitivity is defined by the “lower limit of quantification,” or LLOQ. The LLOQ is the lowest amount of an analyte in a sample that can be quantitatively determined with suitable precision and accuracy. As used herein, “high sensitivity” means that the assay is capable of detecting sub-pg/mL levels of an analyte. In one embodiment, the assay is capable of detecting fg/mL levels of an analyte.

The term “specificity” refers to the ability of an assay to detect only the analyte of interest in the presence of similar or related molecules. As used herein, an assay has “high specificity” when at least 10 samples are tested, or at least 20 samples are tested, or at least 30 samples are tested, or at least 50 samples are tested in the assay and at least 90%, or at least 95%, or 100% of the assay signal in all samples tested is at or below the LLOQ when antigen competition or immunodepletion is performed prior to carrying out the assay as described herein. In some embodiments, the assay is able to detect only the analyte of interest in the presence of one or more unrelated molecules, which may be present at higher concentrations compared to the analyte of interest.

In certain embodiments, the term “at the reference level” refers to a level of the biomarker in the sample from the individual or patient that is essentially identical to the reference level or to a level that differs from the reference level by up to 1%, up to 2%, up to 3%, up to 4%, up to 5%. In some embodiments, the reference level is the median level of the biomarker in a reference population. In some embodiments, a reference level of a marker is the mean level of the marker in a reference population. In some embodiments, a reference level of a marker is the average level of the marker in a reference population. Nonlimiting exemplary reference populations include patients with asthma, patients with moderate to severe asthma, patients with idiopathic pulmonary fibrosis, patients with atopic dermatitis, healthy individuals, and a group including healthy individuals and any of the aforementioned patients.

In certain embodiments, the term “above the reference level” refers to a level of the biomarker in the sample from the individual or patient above the reference level by at least 5%, 10%, 20%, 25%, 30%, 40%, 50%, 60%, 70%, 80%, 85%, 90%, 95%, 100% or greater, determined by the methods described herein, as compared to the reference level. In some embodiments, the reference level is the median level in a reference population. In some embodiments, a reference level of a marker is the mean level of the marker in a reference population. In some embodiments, a reference level of a marker is the average level of the marker in a reference population. Nonlimiting exemplary reference populations include patients with asthma, patients with moderate to severe asthma, patients with idiopathic pulmonary fibrosis, patients with atopic dermatitis, healthy individuals, and a group including healthy individuals and any of the aforementioned patients.

In certain embodiments, the term “below the reference level” refers to a level of the biomarker in the sample from the individual or patient below the reference level by at least 5%, 10%, 20%, 25%, 30%, 40%, 50%, 60%, 70%, 80%, 85%, 90%, 95%, 100% or greater, determined by the methods described herein, as compared to the reference level. In some embodiments, the reference level is the median level in a reference population. In some embodiments, a reference level of a marker is the mean level of the marker in a reference population. In some embodiments, a reference level of a marker is the average level of the marker in a reference population. Nonlimiting exemplary reference populations include patients with asthma, patients with moderate to severe asthma, patients with idiopathic pulmonary fibrosis, patients with atopic dermatitis, healthy individuals, and a group including healthy individuals and any of the aforementioned patients.

The terms “marker” and “biomarker” are used interchangeably to refer to a molecule, including a gene, protein, carbohydrate structure, or glycolipid, metabolite, mRNA, miRNA, protein, DNA (cDNA or genomic DNA), DNA copy number, or an epigenetic change, e.g., increased, decreased, or altered DNA methylation (e.g., cytosine methylation, or CpG methylation, non-CpG methylations); histone modification (e.g., (de)acetylation, (de)methylation, (de)phosphorylation, ubiquitination, SUMOylation, ADP-ribosylation); altered nucleosome positioning, the expression or presence of which in or on a mammalian tissue or cell can be detected by standard methods (or methods disclosed herein) and which may be predictive, diagnostic and/or prognostic for a mammalian cell's or tissue's sensitivity to treatment regimes based on Th2 pathway inhibition using, for example, a Th2 pathway inhibitor described herein. A biomarker may also be a biological or clinical attribute that can be measured in a biological sample obtained from a subject, such as for example but not limited to, blood cell count, e.g., blood eosinophil count, FEV₁ or FeNO. In certain embodiments, the level of such a biomarker is determined to be higher or lower than that observed for a reference population. In certain embodiments, a blood eosinophil count is 200/μl, or 250/μl, or 300/μl, or 400/μl.

The term “comparing” refers to comparing the level of the biomarker in the sample from the individual or patient with the reference level of the biomarker specified elsewhere in this description. It is to be understood that comparing usually refers to a comparison of corresponding parameters or values, e.g., an absolute amount is compared to an absolute reference amount while a concentration is compared to a reference concentration or an intensity signal obtained from the biomarker in a sample is compared to the same type of intensity signal obtained from a reference sample. The comparison may be carried out manually or computer assisted. Thus, the comparison may be carried out by a computing device (e.g., of a system disclosed herein). The value of the measured or detected level of the biomarker in the sample from the individual or patient and the reference level can be, e.g., compared to each other and the said comparison can be automatically carried out by a computer program executing an algorithm for the comparison. The computer program carrying out the said evaluation will provide the desired assessment in a suitable output format. For a computer assisted comparison, the value of the determined amount may be compared to values corresponding to suitable references which are stored in a database by a computer program. The computer program may further evaluate the result of the comparison, i.e. automatically provide the desired assessment in a suitable output format. For a computer assisted comparison, the value of the determined amount may be compared to values corresponding to suitable references which are stored in a database by a computer program. The computer program may further evaluate the result of the comparison, i.e. automatically provides the desired assessment in a suitable output format.

The term “measuring” the level of a biomarker refers to the quantification of the biomarker, e.g. to determining the level of the biomarker in the sample, employing appropriate methods of detection described elsewhere herein.

The term “monitoring the efficacy of a therapy” is used to indicate that a sample is obtained at least once, including serially, from a patient before and/or under therapy and that one or more biomarkers is measured therein to obtain an indication whether the therapy is efficient or not.

In the monitoring of the efficacy of a therapy the levels of one or more biomarkers are measured and in some embodiments compared to a reference level for the biomarkers, or, in some embodiments, are compared to the level of the biomarkers in a sample obtained from the same patient at an earlier point in time. In some embodiments, the current levels of one or more biomarker are compared to the levels of the biomarkers in a sample obtained from the same patient before start of a therapy in said patient.

The phrase “recommending a treatment” refers to using the information or data generated relating to the level or presence of one or more biomarkers described herein in a sample of a patient to identify the patient as suitably treated or not suitably treated with a Th2 pathway inhibitor. The phrase “recommending a treatment” may refer to using the information or data generated for proposing or selecting a therapy comprising a Th2 pathway inhibitor for a patient identified or selected as more or less likely to respond to the therapy comprising a Th2 pathway inhibitor. The information or data used or generated may be in any form, written, oral or electronic. In some embodiments, using the information or data generated includes communicating, presenting, reporting, storing, sending, transferring, supplying, transmitting, dispensing, or combinations thereof. In some embodiments, communicating, presenting, reporting, storing, sending, transferring, supplying, transmitting, dispensing, or combinations thereof are performed by a computing device, analyzer unit or combination thereof. In some further embodiments, communicating, presenting, reporting, storing, sending, transferring, supplying, transmitting, dispensing, or combinations thereof are performed by a laboratory or medical professional. In some embodiments, the information or data includes a comparison of the levels of one or more markers described herein to a reference level. In some embodiments, the information or data includes an indication that the patient is suitably treated or not suitably treated with a therapy comprising a Th2 pathway inhibitor, including, in some instances, an indication that the patient is suitably treated or not suitably treated with a therapy comprising a particular Th2 pathway inhibitor, such as an anti-IL13 antibody or an anti-IgE antibody.

The phrase “selecting a patient” or “identifying a patient” refers to using the information or data generated relating to the levels of one or more markers described herein in a sample of a patient to identify or select the patient as more likely to benefit or less likely to benefit from a therapy comprising a Th2 pathway inhibitor. The information or data used or generated may be in any form, written, oral or electronic. In some embodiments, using the information or data generated includes communicating, presenting, reporting, storing, sending, transferring, supplying, transmitting, dispensing, or combinations thereof. In some embodiments, communicating, presenting, reporting, storing, sending, transferring, supplying, transmitting, dispensing, or combinations thereof are performed by a computing device, analyzer unit or combination thereof. In some further embodiments, communicating, presenting, reporting, storing, sending, transferring, supplying, transmitting, dispensing, or combinations thereof are performed by a laboratory or medical professional. In some embodiments, the information or data includes a comparison of the levels of one or more markers described herein to a reference level. In some embodiments, the information or data includes an indication that the patient is suitably treated or not suitably treated with a therapy comprising a Th2 pathway inhibitor, including, in some instances, an indication that the patient is suitably treated or not suitably treated with a therapy comprising a particular Th2 pathway inhibitor, such as an anti-IL13 antibody or an IgE antibody.

The phrase “selecting a therapy” refers to using the information or data generated relating to the level or presence of one or more markers described herein in a sample of a patient to identify or selecting a therapy for a patient. In some embodiment the therapy may comprise a Th2 pathway inhibitor. The information or data used or generated may be in any form, written, oral or electronic. In some embodiments, using the information or data generated includes communicating, presenting, reporting, storing, sending, transferring, supplying, transmitting, dispensing, or combinations thereof. In some embodiments, communicating, presenting, reporting, storing, sending, transferring, supplying, transmitting, dispensing, or combinations thereof are performed by a computing device, analyzer unit or combination thereof. In some further embodiments, communicating, presenting, reporting, storing, sending, transferring, supplying, transmitting, dispensing, or combinations thereof are performed by a laboratory or medical professional. In some embodiments, the information or data includes an indication that the patient is suitably treated or not suitably treated with a therapy comprising a Th2 pathway inhibitor, including, in some instances, an indication that the patient is suitably treated or not suitably treated with a therapy comprising a particular Th2 pathway inhibitor, such as an anti-IL13 antibody or an IgE antibody.

The term “biological sample” includes, but is not limited to, blood, serum, plasma, peripheral blood mononuclear cells (PBMCs), sputum, tissue biopsies (e.g., lung samples), and nasal samples including nasal swabs or nasal polyps. The sample may be taken before treatment, during treatment or post-treatment. The sample may be taken from a patient who is suspected of having, or is diagnosed as having asthma or a Th2-associated disease, and hence is likely in need of treatment or from a normal individual who is not suspected of having any disorder. In some embodiments, the biological sample is serum. In some embodiments, the biological sample is plasma.

FENO assay refers to an assay that measures FE_{NO} (fractional exhaled nitric oxide) levels. Such levels can be evaluated using, e.g., a hand-held portable device, NIOX MINO® (Aerocrine, Solna, Sweden), in accordance with guidelines published by the American Thoracic Society (ATS) in 2005. FE_{NO} may be noted in other similar ways, e.g., FeNO or FENO, and it should be understood that all such similar variations have the same meaning.

Age of Patients to be tested or treated according to the methods provided herein include: all ages. In some embodiments, the ages are 18+ years old. In some embodiments, the ages are 12+ years old. In some embodiments, the ages are 2+ years old. In some embodiments, the ages are 2-18 years old, 12-18 years old, 18-75 year olds, 12-75 year olds or 2-75 year olds.

Asthma is a complex disorder characterized by variable and recurring symptoms, reversible airflow obstruction (e.g., by bronchodilator) and bronchial hyperresponsiveness which may or may not be associated with underlying inflammation. Examples of asthma include aspirin sensitive/exacerbated asthma, atopic asthma, severe asthma, mild asthma, moderate to severe asthma, corticosteroid naïve asthma, chronic asthma, corticosteroid resistant asthma, corticosteroid refractory asthma, newly diagnosed and untreated asthma, asthma due to smoking, asthma uncontrolled on corticosteroids and other asthmas as mentioned in J Allergy Clin Immunol (2010) 126(5):926-938.

IL-13 mediated disorder means a disorder associated with excess IL-13 levels or activity in which atypical symptoms may manifest due to the levels or activity of IL-13 locally and/or systemically in the body. Examples of IL-13 mediated disorders include: cancers (e.g., non-Hodgkin's lymphoma, glioblastoma), atopic dermatitis, allergic rhinitis, asthma, fibrosis, inflammatory bowel disease (e.g., ulcerative colitis or Crohn's disease), lung inflammatory disorders (e.g., pulmonary fibrosis such as IPF), COPD, hepatic fibrosis.

IL-4 mediated disorder means: a disorder associated with excess IL4 levels or activity in which atypical symptoms may manifest due to the levels or activity of IL4 locally and/or systemically in the body. Examples of IL4 mediated disorders include: cancers (e.g., non-Hodgkin's lymphoma, glioblastoma), atopic dermatitis, allergic rhinitis, asthma, fibrosis, inflammatory bowel disease (e.g., ulcerative colitis or Crohn's disease), lung inflammatory disorders (e.g., pulmonary fibrosis such as IPF), COPD, hepatic fibrosis.

IL-5 mediated disorder means: a disorder associated with excess IL5 levels or activity in which atypical symptoms may manifest due to the levels or activity of IL5 locally and/or systemically in the body. Examples of IL5 mediated disorders include: cancers (e.g., non-Hodgkin's lymphoma, glioblastoma), atopic dermatitis, allergic rhinitis, asthma, fibrosis, inflammatory bowel disease (e.g., ulcerative colitis or Crohn's disease), lung inflammatory disorders (e.g., pulmonary fibrosis such as IPF), COPD, hepatic fibrosis.

IL-9 mediated disorder means: a disorder associated with excess IL9 levels or activity in which atypical symptoms may manifest due to the levels or activity of IL9 locally and/or systemically in the body. Examples of IL9 mediated disorders include: cancers (e.g., non-Hodgkin's lymphoma, glioblastoma), atopic dermatitis, allergic rhinitis, asthma, fibrosis, inflammatory bowel disease (e.g., ulcerative colitis or Crohn's disease), lung inflammatory disorders (e.g., pulmonary fibrosis such as IPF), COPD, hepatic fibrosis.

TSLP mediated disorder means: a disorder associated with excess TSLP levels or activity in which atypical symptoms may manifest due to the levels or activity of TSLP locally and/or systemically in the body. Examples of TSLP mediated disorders include: cancers (e.g., non-Hodgkin's lymphoma, glioblastoma), atopic dermatitis, allergic rhinitis, asthma, fibrosis, inflammatory bowel disease (e.g., ulcerative colitis or Crohn's disease), lung inflammatory disorders (e.g., pulmonary fibrosis such as IPF), COPD, hepatic fibrosis.

IgE-mediated disorder means: a disorder associated with excess IgE levels or activity in which atypical symptoms may manifest due to levels of IgE locally and/or systemically in the body. Such disorders include, asthma, atopic dermatitis, allergic rhinitis, fibrosis (e.g., pulmonary fibrosis, such as IPF).

Asthma-Like Symptom includes a symptom selected from the group consisting of shortness of breath, cough (changes in sputum production and/or sputum quality and/or cough frequency), wheezing, chest tightness, bronchioconstriction and nocturnal awakenings ascribed to one of the symptoms above or a combination of these symptoms (Juniper et al (2000) Am. J. Respir. Crit. Care Med., 162(4), 1330-1334.).

The term “respiratory disorder” include, but is not limited to asthma (e.g., allergic and non-allergic asthma (e.g., due to infection, e.g., with respiratory syncytial virus (RSV), e.g., in younger children)); bronchitis (e.g., chronic bronchitis); chronic obstructive pulmonary disease (COPD) (e.g., emphysema (e.g., cigarette-induced emphysema); conditions involving airway inflammation, eosinophilia, fibrosis and excess mucus production, e.g., cystic fibrosis, pulmonary fibrosis, and allergic rhinitis. Examples of diseases that can be characterized by airway inflammation, excessive airway secretion, and airway obstruction include asthma, chronic bronchitis, bronchiectasis, and cystic fibrosis.

Exacerbations (commonly referred to as asthma attacks or acute asthma) are episodes of new or progressive increase in shortness of breath, cough (changes in sputum production and/or sputum quality and/or cough frequency), wheezing, chest tightness, nocturnal awakenings ascribed to one of the symptoms above or a combination of these symptoms. Exacerbations are often characterized by decreases in expiratory airflow (PEF or FEV₁). However, PEF variability does not usually increase during an exacerbation, although it may do so leading up to or during the recovery from an exacerbation. The severity of exacerbations ranges from mild to life-threatening and can be evaluated based on both symptoms and lung function. Severe asthma exacerbations as described herein include exacerbations that result in any one or combination of the following hospitalization for asthma treatment, high corticosteroid use (e.g., quadrupling the total daily corticosteroid dose or a total daily dose of greater or equal to 500 micrograms of FP or equivalent for three consecutive days or more), or oral/parenteral corticosteroid use.

A Th2 pathway inhibitor, also referred to as a Type 2 pathway inhibitor, is an agent that inhibits the Th2 pathway. Examples of a Th2 pathway inhibitor include inhibitors of the activity of any one of the targets selected from: ITK, BTK, IL-9 (e.g., MEDI-528), IL-5 (e.g., Mepolizumab, CAS No. 196078-29-2; resilizumab), IL-13 (e.g., IMA-026, IMA-638 (also referred to as, anrukinzumab, INN No. 910649-32-0; QAX-576; IL4/IL13 trap), tralokinumab (also referred to as CAT-354, CAS No. 1044515-88-9); AER-001, ABT-308 (also referred to as humanized 13C5.5 antibody), IL-4 (e.g., AER-001, IL4/IL13 trap), IL-17, OX40L, TSLP, IL-25, IL-33, soluble IgE (e.g., XOLAIR, QGE-031; MEDI-4212) and membrane-bound IgE (quilizumab); and receptors such as: IL-9 receptor, IL-5 receptor (e.g., MEDI-563 (benralizumab, CAS No. 1044511-01-4), IL-4receptor alpha (e.g., AMG-317, AIR-645, dupilumab), IL-13receptoralpha1 (e.g., R-1671) and IL-13receptoralpha2, OX40, TSLP-R, IL-7Ralpha (a co-receptor for TSLP), IL17RB (receptor for IL-25), ST2 (receptor for IL-33), CCR3, CCR4, CRTH2 (e.g., AMG-853, AP768, AP-761, MLN6095, ACT129968), FcepsilonRI, FcepsilonRII/CD23 (receptors for IgE), Flap (e.g., GSK2190915), Syk kinase (R-343, PF3526299); CCR4 (AMG-761), TLR9 (QAX-935) and multi-cytokine inhibitor of CCR3, IL5, IL3, GM-CSF (e.g., TPI ASM8). Examples of inhibitors of the aforementioned targets are disclosed in, for example, WO2008/086395; WO2006/085938; U.S. Pat. No. 7,615,213; U.S. Pat. No. 7,501,121; WO2006/085938; WO 2007/080174; U.S. Pat. No. 7,807,788; WO2005007699; WO2007036745; WO2009/009775; WO2007/082068; WO2010/073119; WO2007/045477; WO2008/134724; US2009/0047277; and WO2008/127,271).

A therapeutic agent a provided herein includes an agent that can bind to the target identified herein above, such as a polypeptide(s) (e.g., an antibody, an immunoadhesin or a peptibody), an aptamer or a small molecule that can bind to a protein or a nucleic acid molecule that can bind to a nucleic acid molecule encoding a target identified herein (i.e., siRNA).

“An anti-IL13/IL4 pathway inhibitor” refers to a therapeutic agent that inhibits IL-13 and/or IL-4 signaling. Examples of an anti-IL13/IL4 pathway inhibitors includes inhibitors of the interaction of IL13 and/or IL4 with its receptor(s), such inhibitors include, but are not limited to, anti-IL13 binding agents, anti-IL4 binding agents, anti-IL3/IL4 bispecific binding agents, anti-IL4receptoralpha binding agents, anti-IL13receptoralpha1 binding agents and anti-IL13 receptoralpha2 binding agents. Single domain antibodies that can bind IL13, IL4, (including bispecific antibody with a single domain binding IL13 and a single domain binding IL4), IL-13Ralpha1, IL-13Ralpha2 or IL-4Ralpha are specifically included as inhibitors. It should be understood that molecules that can bind more than one target are included.

“Anti-IL4 binding agents” refers to agent that binds to human IL-4. Such binding agents can include a small molecule, an aptamer or a polypeptide. Such polypeptide can include, but is not limited to, a polypeptide(s) selected from the group consisting of an immunoadhesin, an antibody, a peptibody and a peptide. According to one embodiment, the binding agent binds to a human IL-4 sequence with an affinity between 1 uM-1 pM. Specific examples of anti-IL4 binding agents can include soluble IL4Receptor alpha (e.g., extracellular domain of IL4Receptor fused to a human Fc region), anti-IL4 antibody, and soluble IL13receptoralpha1 (e.g., extracellular domain of IL13receptoralpha1 fused to a human Fc region).

“Anti-IL4receptoralpha binding agents” refers to an agent that binds to human IL4 receptoralpha. Such binding agents can include a small molecule, an aptamer or a polypeptide. Such polypeptide can include, but is not limited to, a polypeptide(s) selected from the group consisting of an immunoadhesin, an antibody, a peptibody and a peptide. According to one embodiment, the binding agent binds to a human IL-4 receptor alpha sequence with an affinity between 1 uM-1 pM. Specific examples of anti-IL4 receptoralpha binding agents can include anti-IL4 receptor alpha antibodies.

“Anti-IL13 binding agent” refers to agent that binds to human IL13. Such binding agents can include a small molecule, aptamer or a polypeptide. Such polypeptide can include, but is not limited to, a polypeptide(s) selected from the group consisting of an immunoadhesin, an antibody, a peptibody and a peptide. According to one embodiment, the binding agent binds to a human IL-13 sequence with an affinity between 1 uM-1 pM. Specific examples of anti-IL13 binding agents can include anti-IL13 antibodies, soluble IL13receptoralpha2 fused to a human Fc, soluble IL4receptoralpha fused to a human Fc, soluble IL13 receptoralpha fused to a human Fc. According to one embodiment, the anti-IL-13 antibody comprises a VH comprising a sequence selected from SEQ ID NOs: 1, 3, and 24, and a VL comprising a sequence selected from SEQ ID NO: 2, 4, and 25. In one embodiment, the anti-IL13 antibody comprises HVRH1, HVRH2, HVRH3, HVRL1, HVRL2, and HVRL3, wherein the respective HVRs have the amino acid sequence of SEQ ID NO.: 5, SEQ ID NO.: 6, SEQ ID NO.: 7, SEQ ID NO.: 8, SEQ ID NO.: 9, and SEQ ID NO.: 10. In one embodiment, the anti-IL-13 antibody is lebrikizumab. According to one embodiment, the antibody is an IgG1 antibody. According to another embodiment, the antibody is an IgG4 antibody. According to one embodiment, the IgG4 antibody comprises a S228P mutation in its constant domain. In one embodiment, the anti-IL-13 antibody comprises a Q1E mutation in its variable heavy chain region. In one embodiment, the anti-IL-13 antibody comprises a M4L mutation in its variable light chain region

Anti-IL13receptoralpha1 binding agents” refers to an agent that specifically binds to human IL13 receptoralpha1. Such binding agents can include a small molecule, aptamer or a polypeptide. Such polypeptide can include, but is not limited to, a polypeptide(s) selected from the group consisting of an immunoadhesin, an antibody, a peptibody and a peptide. According to one embodiment, the binding agent binds to a human IL-13 receptor alpha1 sequence with an affinity between 1 uM-1 pM. Specific examples of anti-IL13 receptoralpha1 binding agents can include anti-IL13 receptor alpha1 antibodies.

“Anti-IL13receptoralpha2 binding agents” refers to an agent that specifically binds to human IL13 receptoralpha2. Such binding agents can include a small molecule, an aptamer or a polypeptide. Such polypeptide can include, but is not limited to, a polypeptide(s) selected from the group consisting of an immunoadhesin, an antibody, a peptibody and a peptide. According to one embodiment, the binding agent binds to a human IL-13 receptor alpha2 sequence with an affinity between 1 μM-1 pM. Specific examples of anti-IL13 receptoralpha2 binding agents can include anti-IL13 receptor alpha2 antibodies.

“Anti IgE binding agents” refers to an agent that specifically binds to human IgE. Such binding agents can include a small molecule, an aptamer or a polypeptide. Such polypeptide can include, but is not limited to, a polypeptide(s) selected from the group consisting of an immunoadhesin, an antibody, a peptibody and a peptide. According to one embodiment, the anti-IgE antibody comprises a variable heavy chain region and a variable light chain region, wherein the variable heavy chain region is SEQ ID NO:22 and the variable light chain region is SEQ ID NO:23. According to one embodiment, the anti-IgE antibody is the XOLAIR® antibody.

A “Th2-associated disease” is used interchangeably herein with “Type 2-associated disease” and is one that involves T-helper type 2 cells (Th2) and inflammation, and which may include other pathological and clinical features such as fibrosis or mucus production, that are associated with Th2 cytokines including, for example, but not limited to IL-4, IL-5, IL-9, and IL-13. Additional immune and/or inflammatory cells and cytokines, enzymes and other inflammatory mediators (e.g., histamines, tryptase, leukotrienes, IgE) produced by such cells may contribute to inflammation and/or disease signs and symptoms. Such additional immune and/or inflammatory cells include, but are not limited to, Th17 cells, type 2 innate lymphoid cells, eosinophils, mast cells, basophils, neutrophils, and IgE-producing B cells. Examples of Th2-associated diseases (also referred to herein as Type 2-associated diseases) include asthma, atopic asthma, allergic asthma, severe asthma, atopic dermatitis, allergic rhinitis (including seasonal allergic rhinitis), food hypersensitivity, urticaria, bullous skin diseases, chronic eosinophilic pneumonia, allergic bronchopulmonary aspergillosis, celiac disease, Churg-Strauss syndrome (periarteritis nodosa plus atopy), eosinophilic myalgia syndrome, hypereosinophilic syndrome, edematous reactions including episodic angiodema, eosinophilic esophagitis, eosinophilic gastritis, eosinophilic gastroenteritis, eosinophilic enteritis and eosinophilic colitis, nasal micropolyposis and polyposis, inflammatory bowel disease (e.g., ulcerative colitis and Crohn's disease), scleroderma, fibrosis, idiopathic pulmonary fibrosis (IPF), lung inflammatory disorders, chronic obstructive pulmonary disease (COPD), hepatic fibrosis, endomyocardial fibrosis, chronic bronchitis, bronchiectasis, cystic fibrosis and malignancies, for example, cancers or tumors associated with aberrant expression of a Th2 cytokine, such as IL-13.

The term “small molecule” refers to an organic molecule having a molecular weight between 50 Daltons to 2500 Daltons.

The term “antibody” is used in the broadest sense and specifically covers, for example, monoclonal antibodies, polyclonal antibodies, antibodies with polyepitopic specificity, single chain antibodies, multi-specific antibodies, including bispecific antibodies, and antibody fragments so long as they exhibit the desired antigen-binding activity. Such antibodies can be chimeric, humanized, human and synthetic.

The term “uncontrolled” or “uncontrollable” refers to the inadequacy of a treatment regimen to minimize a symptom of a disease. As used herein, the term “uncontrolled” and “inadequately controlled” can be used interchangeably and are meant to refer to the same state. The control status of a patient can be determined by the attending physician based on a number of factors including the patient's clinical history, responsiveness to treatment and level of current treatment prescribed. For example, a physician may consider factors such as FEV₁<75% predicted or personal best, frequency of need for a SABA in the past 2-4 weeks (e.g., greater than or equal two doses/week), nocturnal awakenings/symptoms in the past 2-4 weeks (e.g., less than or equal to 2 nights/week), limitations on activity in the past 2-4 weeks, daytime symptoms in the past 2-4 weeks

The term “therapeutic agent” refers to any agent that is used to treat a disease.

The term “controller” or “preventor” refers to any therapeutic agent that is used to control asthma inflammation. Examples of controllers include corticosteroids, leukotriene receptor antagonists (e.g., inhibit the synthesis or activity of leukotrienes such as montelukast, zileuton, pranlukast, zafirlukast), LABAs, corticosteroid/LABA combination compositions, theophylline (including aminophylline), cromolyn sodium, nedocromil sodium, omalizumab, LAMAs, MABA (e.g, bifunctional muscarinic antagonist-beta2 Agonist), 5-Lipoxygenase Activating Protein (FLAP) inhibitors, and enzyme PDE-4 inhibitor (e.g., roflumilast). A “second controller” typically refers to a controller that is not the same as the first controller.

The term “corticosteroid sparing” or “CS” means the decrease in frequency and/or amount, or the elimination of, corticosteroid used to treat a disease in a patient taking corticosteroids for the treatment of the disease due to the administration of another therapeutic agent. A “CS agent” refers to a therapeutic agent that can cause CS in a patient taking a corticosteroid.

The term “corticosteroid” includes, but is not limited to fluticasone (including fluticasone propionate (FP)), beclometasone, budesonide, ciclesonide, mometasone, flunisolide, betamethasone and triamcinolone. “Inhalable corticosteroid” means a corticosteroid that is suitable for delivery by inhalation. Exemplary inhalable corticosteroids are fluticasone, beclomethasone dipropionate, budenoside, mometasone furoate, ciclesonide, flunisolide, triamcinolone acetonide and any other corticosteroid currently available or becoming available in the future. Examples of corticosteroids that can be inhaled and are combined with a long-acting beta2-agonist include, but are not limited to: budesonide/formoterol and fluticasone/salmeterol.

Examples of corticosteroid/LABA combination drugs include fluticasone furoate/vilanterol trifenatate and indacaterol/mometasone.

The term “LABA” means long-acting beta-2 agonist, which agonist includes, for example, salmeterol, formoterol, bambuterol, albuterol, indacaterol, arformoterol and clenbuterol.

The term “LAMA” means long-acting muscarinic antagonist, which agonists include: tiotropium.

Examples of LABA/LAMA combinations include, but are not limited to: olodaterol tiotropium (Boehringer Ingelheim's) and indacaterol glycopyrronium (Novartis)

The term “SABA” means short-acting beta-2 agonists, which agonists include, but are not limited to, salbutamol, levosalbutamol, fenoterol, terbutaline, pirbuterol, procaterol, bitolterol, rimiterol, carbuterol, tulobuterol and reproterol

Leukotriene receptor antagonists (sometimes referred to as a leukast) (LTRA) are drugs that inhibit leukotrienes. Examples of leukotriene inhibitors include montelukast, zileuton, pranlukast, and zafirlukast.

The term “FEV₁” refers to the volume of air exhaled in the first second of a forced expiration. It is a measure of airway obstruction. Provocative concentration of methacholine required to induce a 20% decline in FEV₁ (PC20) is a measure of airway hyperresponsiveness. FEV₁ may be noted in other similar ways, e.g., FEV₁, and it should be understood that all such similar variations have the same meaning.

The term “relative change in FEV₁”=(FEV₁ at week 12 of treatment−FEV₁ prior to start of treatment) divided by FEV₁

The term “mild asthma” refers to a patient generally experiencing symptoms or exacerbations less than two times a week, nocturnal symptoms less than two times a month, and is asymptomatic between exacerbations. Mild, intermittent asthma is often treated as needed with the following: inhaled bronchodilators (short-acting inhaled beta2-agonists); avoidance of known triggers; annual influenza vaccination; pneumococcal vaccination every 6 to 10 years, and in some cases, an inhaled beta2-agonist, cromolyn, or nedocromil prior to exposure to identified triggers. If the patient has an increasing need for short-acting beta2-agonist (e.g., uses short-acting beta2-agonist more than three to four times in 1 day for an acute exacerbation or uses more than one canister a month for symptoms), the patient may require a stepup in therapy.

The term “moderate asthma” generally refers to asthma in which the patient experiences exacerbations more than two times a week and the exacerbations affect sleep and activity; the patient has nighttime awakenings due to asthma more than two times a month; the patient has chronic asthma symptoms that require short-acting inhaled beta2-agonist daily or every other day; and the patient's pretreatment baseline PEF or FEV₁ is 60 to 80 percent predicted and PEF variability is 20 to 30 percent.

The term “severe asthma” generally refers to asthma in which the patient has almost continuous symptoms, frequent exacerbations, frequent nighttime awakenings due to the asthma, limited activities, PEF or FEV₁ baseline less than 60 percent predicted, and PEF variability of 20 to 30 percent.

Examples of rescue medications include albuterol, ventolin and others.

“Resistant” refers to a disease that demonstrates little or no clinically significant improvement after treatment with a therapeutic agent. For example, asthma which requires treatment with high dose ICS (e.g., quadrupling the total daily corticosteroid dose or a total daily dose of greater or equal to 500 micrograms of FP (or equivalent) for at least three consecutive days or more, or systemic corticosteroid for a two week trial to establish if asthma remains uncontrolled or FEV₁ does not improve is often considered severe refractory asthma.

A therapeutic agent as provided herein can be administered by any suitable means, including parenteral, subcutaneous, intraperitoneal, intrapulmonary, and intranasal. Parenteral infusions include intramuscular, intravenous, intraarterial, intraperitoneal, or subcutaneous administration. In one embodiment, the therapeutic agent is inhaled. According to another embodiment, the dosing is given by injections, e.g., intravenous or subcutaneous injections. In yet another embodiment, the therapeutic agent is administered using a syringe (e.g., prefilled or not) or an autoinjector.

For the prevention or treatment of disease, the appropriate dosage of a therapeutic agent may depend on the type of disease to be treated, the severity and course of the disease, whether the therapeutic agent is administered for preventive or therapeutic purposes, previous therapy, the patient's clinical history and response to the therapeutic agent, and the discretion of the attending physician. The therapeutic agent is suitably administered to the patient at one time or over a series of treatments. The therapeutic agent composition will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

Dosing for lebrikizumab, for Th2-associated diseases (including asthma) and for treating other diseases using Th2 therapies: lebrikizumab can be administered 0.1 mg/kg to 100 mg/kg of the patient's body weight. In one embodiment, the dosage administered to a patient is between 0.1 mg/kg and 20 mg/kg of the patient's body weight. In another embodiment, the dose is 1 mg/kg to 10 mg/kg of the patient's body weight.

In an alternative embodiment, lebrikizumab can be administered as a flat dose. In one embodiment lebrikizumab is administered as a flat dose (i.e., not weight dependent) of between 125-1000 mg, or a flat dose of 37.5 mg, or a flat dose of 125 mg, or a flat dose of 250 mg, or a flat dose of 500 mg, by subcutaneous injection or by intravenous injection, at a frequency of time selected from the group consisting of: every 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 1 month, 2 months, 3 month or 4 months. In another embodiment, if the patient is overweight, lebrikizumab can be administered, e.g., 125-250 mg at a frequency of 3 times per month. In one embodiment, the lebrikizumab is administered as a flat dose of 125 mg, 250 mg or 500 mg every 4 weeks. In another embodiment, the lebrikizumab is administered in a patient >40 kg as a flat dose of 37.5 mg, 125 mg, 250 mg or 500 mg every 4 weeks.

In one embodiment, the patient is 18 years of age or older. In one embodiment, the asthma patient is age 12 to 17 and lebrikizumab is administered in as a flat dose of 250 mg or a flat dose of 125 mg. In one embodiment, the asthma patient is age 6 to 11 and lebrikizumab is administered in as a flat dose of 125 mg.

“Patient response” or “response” (and grammatical variations thereof) to a therapeutic agent can be assessed using any endpoint indicating a benefit to the patient, including, without limitation, (1) inhibition, to some extent, of disease progression, including slowing down and complete arrest; (2) reduction in the number of disease episodes and/or symptoms; (3) reduction in lesional size; (4) inhibition (i.e., reduction, slowing down or complete stopping) of immune or inflammatory cell infiltration into adjacent peripheral organs and/or tissues; (5) inhibition (i.e. reduction, slowing down or complete stopping) of disease spread; (6) decrease of auto-immune response, which may, but does not have to, result in the regression or ablation of the disease lesion; (7) relief, to some extent, of one or more symptoms associated with the disorder; (8) increase in the length of disease-free presentation following treatment; and/or (9) decreased mortality at a given point of time following treatment.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen binding arm). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The terms “anti-target antibody” and “an antibody that binds to target” refer to an antibody that is capable of binding the target with sufficient affinity such that the antibody is useful as a diagnostic and/or therapeutic agent in targeting the target. In one embodiment, the extent of binding of an anti-target antibody to an unrelated, non-target protein is less than about 10% of the binding of the antibody to target as measured, e.g., by a radioimmunoassay (RIA) or biacore assay. In certain embodiments, an antibody that binds to a target has a dissociation constant (Kd) of ≤1 μM, ≤100 nM, ≤10 nM, ≤1 nM, ≤0.1 nM, ≤0.01 nM, or ≤0.001 nM (e.g. 10-8 M or less, e.g. from 10-8 M to 10-13 M, e.g., from 10-9 M to 10-13 M). In certain embodiments, an anti-target antibody binds to an epitope of a target that is conserved among different species.

An “antibody fragment” refers to a molecule other than an intact antibody that comprises a portion of an intact antibody that binds the antigen to which the intact antibody binds. Examples of antibody fragments include but are not limited to single chain Fv, Fab, Fab′, Fab′-SH, F(ab′)2; diabodies; linear antibodies; single-chain antibody molecules (e.g. scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. Various methods for carrying out competition assays are well-known in the art.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

An “effective amount” of an agent, e.g., a pharmaceutical formulation, refers to an amount effective, at dosages and for periods of time necessary, to achieve the desired therapeutic or prophylactic result.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter typically being of highest sequence variability and/or involved in antigen recognition. An HVR region as used herein comprise any number of residues located within positions 24-36 (for HVRL1), 46-56 (for HVRL2), 89-97 (for HVRL3), 26-35B (for HVRH1), 47-65 (for HVRH2), and 93-102 (for HVRH3).

An “individual” or “patient” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or patient or subject is a human. In some embodiments, an “individual” or “patient” or “subject” herein is any single human subject eligible for treatment who is experiencing or has experienced one or more signs, symptoms, or other indicators of asthma or a respiratory condition. Intended to be included as a subject are any subjects involved in clinical research trials not showing any clinical sign of disease, or subjects involved in epidemiological studies, or subjects once used as controls. The subject may have been previously treated with a Th2 pathway inhibitor or another drug, or not so treated. The subject may be naïve to a Th2 inhibitor when the treatment herein is started, i.e., the subject may not have been previously treated with, for example, a Th2 inhibitor at “baseline” (i.e., at a set point in time before the administration of a first dose of a Th2 inhibitor in the treatment method herein, such as the day of screening the subject before treatment is commenced). Such “naïve” subjects are generally considered to be candidates for treatment with such drug(s).

A “pediatric” individual or patient or subject is a human from birth to 18 years old (or 0 to 18 years old). In some embodiments, a pediatric individual or patient or subject is from 2 to 6, 2 to 17, 6 to 11, 6 to 18, 6 to 17, 8 to 17, 12 to 17, or 12 to 18 years old.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-target antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “monoclonal antibody” refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used according to the methods provided herein may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “package insert” is used to refer to instructions customarily included in commercial packages of therapeutic products, that contain information about the indications, usage, dosage, administration, combination therapy, contraindications and/or warnings concerning the use of such therapeutic products. The term “package insert” is also used to refer to instructions customarily included in commercial packages of diagnostic products that contain information about the intended use, test principle, preparation and handling of reagents, specimen collection and preparation, calibration of the assay and the assay procedure, performance and precision data such as sensitivity and specificity of the assay.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “target” refers to any native molecule from any vertebrate source, including mammals such as primates (e.g. humans) and rodents (e.g., mice and rats), unless otherwise indicated. The term encompasses “full-length,” unprocessed target as well as any form of target that results from processing in the cell. The term also encompasses naturally occurring variants of targets, e.g., splice variants or allelic variants.

The term “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector” refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### Compositions and Methods

The invention provides, at least in part, IL-13 immunoassay methods that are highly sensitive, detecting femtogram/mL levels of IL-13 in greater than 98% of samples tested, and are highly specific, as described herein. Also provided herein are methods of using such highly sensitive and highly specific immunoassay methods to select or identify patients with elevated serum IL-13 levels who are more likely to respond to therapeutic treatments that are Th2 pathway inhibitors as well as to identify asthma patients who are more likely to suffer from severe exacerbations.

### Exemplary Antibodies

### Anti-IL13 Antibodies

In one aspect, the invention provides isolated antibodies that bind to human IL-13.

Exemplary anti-IL13 antibodies are known and include, for example, but not limited to, lebrikizumab, IMA-026, IMA-638 (also referred to as, anrukinzumab, INN No. 910649-32-0; QAX-576), tralokinumab (also referred to as CAT-354, CAS No. 1044515-88-9); AER-001, ABT-308 (also referred to as humanized 13C5.5 antibody. Examples of such anti-IL13 antibodies and other inhibitors of IL13 are disclosed, for example, in WO 2005/062967, WO2008/086395, WO2006/085938, U.S. Pat. No. 7,615,213, U.S. Pat. No. 7,501,121, WO2007/036745, WO2010/073119, WO2007/045477, WO 2014/165771. In one embodiment, the anti-IL13 antibody is a humanized IgG4 antibody. In one embodiment, the anti-IL13 antibody is lebrikizumab. In one embodiment, the anti-IL13 antibody comprises three heavy chain HVRs, HVR-H1 (SEQ ID NO.: 5), HVR-H2 (SEQ ID NO.: 6), and HVR-H3 (SEQ ID NO.: 7). In one embodiment, the anti-IL13 antibody comprises three light chain HVRS, HVR-L1 (SEQ ID NO.: 8), HVR-L2 (SEQ ID NO.: 9), and HVR-L3 (SEQ ID NO.: 10). In one embodiment, the anti-IL13 antibody comprises three heavy chain HVRs and three light chain HVRs, HVR-H1 (SEQ ID NO.: 5), HVR-H2 (SEQ ID NO.: 6), HVR-H3 (SEQ ID NO.: 7), HVR-L1 (SEQ ID NO.: 8), HVR-L2 (SEQ ID NO.: 9), and HVR-L3 (SEQ ID NO.: 10). In one embodiment, the anti-IL13 antibody comprises a variable heavy chain region, VH, having an amino acid sequence selected from SEQ ID NOs. 1, 3, and 24. In one embodiment, the anti-IL13 antibody comprises a variable light chain region, VL, having an amino acid sequence selected from SEQ ID NOs.: 2, 4, and 25. In one embodiment, the anti-IL13 antibody comprises a variable heavy chain region, VH, having an amino acid sequence selected from SEQ ID NOs. 1, 3, and 24 and a variable light chain region, VL, having an amino acid sequence selected from SEQ ID NOs.: 2, 4, and 25.

In another embodiment, the antibody comprises the variable region sequences SEQ ID NO:1 and SEQ ID NO:2. In another embodiment, the antibody comprises the variable region sequences SEQ ID NO:1 and SEQ ID NO:4. In another embodiment, the antibody comprises the variable region sequences SEQ ID NO:1 and SEQ ID NO:25. In another embodiment, the antibody comprises the variable region sequences SEQ ID NO:3 and SEQ ID NO:2. In another embodiment, the antibody comprises the variable region sequences SEQ ID NO:3 and SEQ ID NO:4. In another embodiment, the antibody comprises the variable region sequences SEQ ID NO:3 and SEQ ID NO:25. In another embodiment, the antibody comprises the variable region sequences SEQ ID NO:24 and SEQ ID NO:2. In another embodiment, the antibody comprises the variable region sequences SEQ ID NO:24 and SEQ ID NO:4. In another embodiment, the antibody comprises the variable region sequences SEQ ID NO:24 and SEQ ID NO:25.

In any of the above embodiments, an anti-IL-13 antibody can be humanized. In one embodiment, an anti-IL-13 antibody comprises HVRs as in any of the above embodiments, and further comprises an acceptor human framework, e.g. a human immunoglobulin framework or a human consensus framework.

In another aspect, an anti-IL-13 antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:1. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-IL-13 antibody comprising that sequence retains the ability to bind to human IL-13. In certain embodiments, a total of 1 to 10 amino acids have been substituted, altered inserted and/or deleted in SEQ ID NO: 1. In certain embodiments, substitutions, insertions, or deletions occur in regions outside the HVRs (i.e., in the FRs). Optionally, the anti-IL13 antibody comprises the VH sequence in SEQ ID NO: 1, including post-translational modifications of that sequence. Optionally, the anti-IL13 antibody comprises the VH sequence in SEQ ID NO: 3, including post-translational modifications of that sequence. Optionally, the anti-IL13 antibody comprises the VH sequence in SEQ ID NO: 24, including post-translational modifications of that sequence.

In another aspect, an anti-IL-13 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:2. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-IL-13 antibody comprising that sequence retains the ability to bind to IL-13. In certain embodiments, a total of 1 to 10 amino acids have been substituted, inserted and/or deleted in SEQ ID NO:2. In certain embodiments, the substitutions, insertions, or deletions occur in regions outside the HVRs (i.e., in the FRs). Optionally, the anti-IL-13 antibody comprises the VL sequence in SEQ ID NO:2, including post-translational modifications of that sequence. Optionally, the anti-IL-13 antibody comprises the VL sequence in SEQ ID NO: 4, including post-translational modifications of that sequence. Optionally, the anti-IL-13 antibody comprises the VL sequence in SEQ ID NO: 25, including post-translational modifications of that sequence.

In another aspect, an anti-IL-13 antibody is provided, wherein the antibody comprises a VH as in any of the embodiments provided above, and a VL as in any of the embodiments provided above.

In a further aspect, the invention provides an antibody that binds to the same epitope as an anti-IL-13 antibody provided herein. For example, in certain embodiments, an antibody is provided that binds to the same epitope as or can by competitively inhibited by an anti-IL-13 antibody comprising a VH sequence of SEQ ID NO:1 and a VL sequence of SEQ ID NO:2.

In a further aspect of the invention, an anti-IL-13 antibody according to any of the above embodiment can be a monoclonal antibody, including a chimeric, humanized or human antibody. In one embodiment, an anti-IL13 antibody is an antibody fragment, e.g., a Fv, Fab, Fab′, scFv, diabody, or F(ab′)2 fragment. In another embodiment, the antibody is a full length antibody, e.g., an intact IgG1 or IgG4 antibody or other antibody class or isotype as defined herein. According to another embodiment, the antibody is a bispecific antibody. In one embodiment, the bispecific antibody comprises the HVRs or comprises the VH and VL regions described above.

In one embodiment, the anti-IL13 antibody comprises three heavy chain HVRs, HVR-H1 (SEQ ID NO.: 13), HVR-H2 (SEQ ID NO.: 14), and HVR-H3 (SEQ ID NO.: 15). In one embodiment, the anti-IL13 antibody comprises three light chain HVRS, HVR-L1 (SEQ ID NO.: 16), HVR-L2 (SEQ ID NO.: 17), and HVR-L3 (SEQ ID NO.: 18). In one embodiment, the anti-IL13 antibody comprises three heavy chain HVRs and three light chain HVRs, HVR-H1 (SEQ ID NO.: 13), HVR-H2 (SEQ ID NO.: 14), HVR-H3 (SEQ ID NO.: 15), HVR-L1 (SEQ ID NO.: 16), HVR-L2 (SEQ ID NO.: 17), and HVR-L3 (SEQ ID NO.: 18). In one embodiment, the anti-IL13 antibody comprises a variable heavy chain region, VH, having the amino acid sequence of SEQ ID NO: 12. In one embodiment, the anti-IL13 antibody comprises a variable light chain region, VL, having the amino acid sequence of SEQ ID NO: 11. In one embodiment, the anti-IL13 antibody comprises a variable heavy chain region, VH, having the amino acid sequence of SEQ ID NO: 12 and a variable light chain region, VL, having the amino acid sequence of SEQ ID NO: 11.

In any of the above embodiments, an anti-IL-13 antibody can be humanized. In one embodiment, an anti-IL-13 antibody comprises HVRs as in any of the above embodiments, and further comprises an acceptor human framework, e.g. a human immunoglobulin framework or a human consensus framework.

In another aspect, an anti-IL-13 antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:12. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-IL-13 antibody comprising that sequence retains the ability to bind to human IL-13. In certain embodiments, a total of 1 to 10 amino acids have been substituted, altered inserted and/or deleted in SEQ ID NO: 12. In certain embodiments, substitutions, insertions, or deletions occur in regions outside the HVRs (i.e., in the FRs). Optionally, the anti-IL13 antibody comprises the VH sequence in SEQ ID NO: 12, including post-translational modifications of that sequence.

In another aspect, an anti-IL-13 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:11. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-IL-13 antibody comprising that sequence retains the ability to bind to IL-13. In certain embodiments, a total of 1 to 10 amino acids have been substituted, inserted and/or deleted in SEQ ID NO:11. In certain embodiments, the substitutions, insertions, or deletions occur in regions outside the HVRs (i.e., in the FRs). Optionally, the anti-IL-13 antibody comprises the VL sequence in SEQ ID NO:11, including post-translational modifications of that sequence.

In another aspect, an anti-IL-13 antibody is provided, wherein the antibody comprises a VH as in any of the embodiments provided above, and a VL as in any of the embodiments provided above.

In a further aspect, the invention provides an antibody that binds to the same epitope as an anti-IL-13 antibody provided herein. For example, in certain embodiments, an antibody is provided that binds to the same epitope as or can by competitively inhibited by an anti-IL-13 antibody comprising a VH sequence of SEQ ID NO:12 and a VL sequence of SEQ ID NO:11.

In a further aspect of the invention, an anti-IL-13 antibody according to any of the above embodiment can be a monoclonal antibody, including a chimeric, humanized or human antibody. In one embodiment, an anti-IL13 antibody is an antibody fragment, e.g., a Fv, Fab, Fab′, scFv, diabody, or F(ab′)2 fragment. In another embodiment, the antibody is a full length antibody, e.g., an intact IgG1 or IgG4 antibody or other antibody class or isotype as defined herein. According to another embodiment, the antibody is a bispecific antibody. In one embodiment, the bispecific antibody comprises the HVRs or comprises the VH and VL regions described above.

In a further aspect, an anti-IL-13 antibody according to any of the above embodiments may incorporate any of the features, singly or in combination, as described in Sections 1-7 below:

### Anti-IgE Antibodies

In one aspect, the invention provides an anti-IgE antibody comprising a variable heavy chain region comprising the amino acid sequence of SEQ ID NO:22 and a variable light chain region comprising the amino acid sequence of SEQ ID NO:23. According to one embodiment, the anti-IgE antibody is the XOLAIR® antibody.

### Bispecific Antibodies

In one aspect, the invention provides a bispecific antibody comprising an antigen-binding domain that specifically binds to IL-4 and IL-13. Such anti-IL-4/anti-IL-13 bispecific antibodies are described in WO 2014/165771.

In another aspect, the invention provides a bispecific antibody comprising an antigen-binding domain that specifically binds to IL-13 and IL-17. Such anti-IL-13/anti-IL-17 bispecific antibodies are described in PCT/US2015/017168 and U.S. application Ser. No. 14/629,449. In some embodiments, the anti-IL-17 antibody binds IL-17A homodimer, IL-17F homodimer, and IL-17AF homodimer.

In a further aspect, the anti-IL-13/anti-IL-17 bispecific antibody comprises an anti-IL-13 VH/VL unit comprising HVRH1, HVRH2, HVRH3, HVRL1, HVRL2, and HVRL3, wherein the respective HVRs have the amino acid sequence of SEQ ID NO.: 5, SEQ ID NO.: 6, SEQ ID NO.: 7, SEQ ID NO.: 8, SEQ ID NO.: 9, and SEQ ID NO.: 10; and an anti-IL-17 VH/VL unit comprising HVRH1, HVRH2, HVRH3, HVRL1, HVRL2, and HVRL3, wherein the respective HVRs have the amino acid sequence of SEQ ID NO.: 26, SEQ ID NO.: 27, SEQ ID NO.: 28, SEQ ID NO.: 29, SEQ ID NO.: 30, and SEQ ID NO.: 31.

In a further aspect, anti-IL-13/anti-IL-17 bispecific antibody comprises an anti-IL-13 VH/VL unit comprising a VH comprising an amino acid sequence selected from SEQ ID NOs: 1, 3, and 24, and a VL comprising an amino acid sequence selected from SEQ ID NO: 2, 4, and 25; and an anti-IL-17 VH/VL unit comprising a VH comprising the amino acid sequence of SEQ ID NO: 32 and a VL comprising the amino acid sequence of SEQ ID NO: 33.

In a further aspect, an anti-IL-13/anti-IL-17 bispecific antibody according to any of the above embodiments may incorporate any of the features, singly or in combination, as described in Sections 1-7 below:

### 1. Antibody Affinity

In certain embodiments, an antibody provided herein has a dissociation constant (Kd) of ≤1 μM, ≤100 nM, ≤10 nM, ≤1 nM, ≤0.1 nM, ≤0.01 nM, or ≤0.001 nM (e.g. 10-8 M or less, e.g. from 10-8 M to 10-13 M, e.g., from 10-9 M to 10-13 M).

In one embodiment, Kd is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (125I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [125I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20 ™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using surface plasmon resonance assays using a BIACORE®-2000 or a BIACORE®-3000 (BIAcore, Inc., Piscataway, N.J.) at 25° C. with immobilized antigen CMS chips at ˜10 response units (RU). Briefly, carboxymethylated dextran biosensor chips (CMS, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kon) and dissociation rates (koff) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio koff/kon. See, e.g., Chen et al., J. Mol. Biol. 293:865-881 (1999). If the on-rate exceeds 106 M−1 s−1 by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

### 2. Antibody Fragments

In certain embodiments, an antibody provided herein is an antibody fragment. Antibody fragments include, but are not limited to, Fab, Fab′, Fab′-SH, F(ab′)2, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

### 3. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 4. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### 5. Library-Derived Antibodies

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

### 6. Multispecific Antibodies

In certain embodiments, an antibody provided herein is a multispecific antibody, e.g. a bispecific antibody. Multispecific antibodies are monoclonal antibodies that have binding specificities for at least two different sites. In certain embodiments, one of the binding specificities is for IL-13 and the other is for any other antigen. In certain embodiments, bispecific antibodies may bind to two different epitopes of IL-13. Bispecific antibodies may also be used to localize cytotoxic agents to cells. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bispecific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting FAb” or “DAF” comprising an antigen binding site that binds to IL-13 as well as another, different antigen (see, US 2008/0069820, for example).

### 7. Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 1 under the heading of “conservative substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1

| Original | Exemplary | Conservative |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

### Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

### Fe Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (Cell Technology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. No. 5,648,260; U.S. Pat. No. 5,624,821; and WO 94/29351 concerning other examples of Fc region variants.

### Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and 5400 (EU numbering) of the heavy chain Fc region. Cysteine engineered antibodies may be generated as described, e.g., in U.S. Pat. No. 7,521,541.

### Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### Recombinant Methods and Compositions

Antibodies may be produced using recombinant methods and compositions, e.g., as described in U.S. Pat. No. 4,816,567. In one embodiment, isolated nucleic acid encoding an antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

### Methods and Compositions for Diagnostics and Detection

The present invention is based, at least in part, on IL-13 immunoassay methods that are highly sensitive, detecting femtogram/mL levels of IL-13 in greater than 98% of samples tested, and are highly specific, as described herein. Also provided herein are methods of using such highly sensitive and highly specific immunoassay methods to select or identify patients with elevated serum IL-13 levels who are more likely to respond to therapeutic treatments that are Th2 pathway inhibitors as well as to identify asthma patients who are more likely to suffer from severe exacerbations.

Accordingly, in one aspect, high sensitivity and high specificity immunoassay methods for detecting and quantifying IL-13 in samples are provided. In certain embodiments, the samples are biological samples. In certain embodiments, the samples are serum. In certain embodiments, the samples are human serum. In some embodiments, the sensitivity is determined as a lower limit of quantification (LLOQ). In certain embodiments, the LLOQ is between 0.1 fg/mL and 35 fg/mL or between about 0.1 fg/mL and about 35 fg/mL. In certain embodiments, the LLOQ between 1 fg/mL and 30 fg/mL or between about 1 fg/mL and about 30 fg/mL. In certain embodiments, the LLOQ is between 5 fg/mL and 25 fg/mL or between about 5 fg/mL and about 25 fg/mL. In certain embodiments, the LLOQ is between 10 fg/mL and 20 fg/mL or between about 10 fg/mL and about 20 fg/mL. In certain embodiments, the LLOQ is 14 fg/mL.

In another aspect, sandwich immunoassay methods are provided that comprise a first monoclonal capture antibody that specifically binds IL-13 and a second monoclonal detection antibody that specifically binds IL-13, wherein the first antibody binds a different epitope than the second antibody. In some embodiments, the specificity is determined by an antigen depletion method (also referred to as an immunodepletion method) which comprises incubation of the sample with an excess amount of the first antibody prior to performing the immunoassay method. In certain such embodiments, antigen in the sample is completely depleted thereby producing a signal below the LLOQ in the immunoassay method. In some embodiments, the sample comprises soluble IL-13Rα2 and the soluble IL-13Rα2 does not interfere with the sensitivity or specificity of the immunoassay method.

In yet another aspect, the immunoassay methods comprise a first antibody comprising a variable region comprising a variable heavy chain region comprising HVR-H1 comprising the amino acid sequence of SEQ ID NO: 5, HVR-H2 comprising the amino acid sequence of SEQ ID NO: 6, and HVR-H3 comprising the amino acid sequence of SEQ ID NO: 7 and a variable light chain region comprising HVR-L1 comprising the amino acid sequence of SEQ ID NO: 8, HVR-L2 comprising the amino acid sequence of SEQ ID NO: 9, and HVR-L3 comprising the amino acid sequence of SEQ ID NO: 10. In some embodiments, the first antibody comprises a variable region comprising a variable heavy chain region comprising the amino acid sequence of SEQ ID NO: 1 and a variable light chain region comprising the amino acid sequence of SEQ ID NO: 2. In certain embodiments, the first antibody is an antibody fragment. In certain embodiments, the first antibody is an antibody fragment which is F(ab′)₂ or Fab. In certain embodiments, the first antibody is an antibody fragment which is Fab, F(ab′)₂, Fab′, or Fv. In some embodiments, the immunoassay methods comprise a second antibody comprising a variable region comprising a variable heavy chain region comprising HVR-H1 comprising the amino acid sequence of SEQ ID NO: 13, HVR-H2 comprising the amino acid sequence of SEQ ID NO: 14, and HVR-H3 comprising the amino acid sequence of SEQ ID NO: 15 and a variable light chain region comprising HVR-L1 comprising the amino acid sequence of SEQ ID NO: 16, HVR-L2 comprising the amino acid sequence of SEQ ID NO: 17, and HVR-L3 comprising the amino acid sequence of SEQ ID NO: 18. In some embodiments, the second antibody comprises a variable region comprising a variable heavy chain region comprising the amino acid sequence of SEQ ID NO: 12 and a variable light chain region comprising the amino acid sequence of SEQ ID NO: 11.

In yet still another aspect, the immunoassay methods further comprise a third antibody, wherein the third antibody specifically binds to the second antibody and is detectably labeled. In some embodiments, the second antibody is labeled with a hapten and the third antibody is an anti-hapten antibody. In some embodiments, the hapten is digoxigenen and the anti-hapten antibody is an anti-digoxigenin monoclonal antibody conjugated with fluorescent latex.

The present invention is also based at least in part on the use of circulating IL-13 to identify subjects more or less likely to respond to therapeutic treatment with a Th2 pathway inhibitor. Thus, the disclosed methods provide convenient, efficient, and potentially cost-effective means to obtain data and information useful in assessing appropriate or effective therapies for treating patients. For example, a sample can be obtained from an asthma patient or a Th2-associated disease patient, and the sample can be examined by the highly sensitive and highly specific IL-13 assay described herein to measure IL-13 and determine whether the expression level of IL-13 has increased or decreased as compared to the expression level in a reference population. In some embodiments, if expression levels of circulating IL-13 in the sample from the patient is greater than or equal to the expression level in a healthy individual, then the patient is likely to benefit from treatment with a Th2 pathway inhibitor.

In certain embodiments, the samples are normalized for both differences in the amount of protein assayed and variability in the quality of the protein samples used, and variability between assay runs. Normalized expression levels for a protein per tested sample per patient can be expressed as a percentage of the expression level measured in the reference set. The expression level measured in a particular patient sample to be analyzed will fall at some percentile within this range, which can be determined by methods known in the art.

A biological sample comprising a biomarker can be obtained by methods known in the art. In addition, the progress of therapy can be monitored more easily by testing such body samples for target genes or gene products.

Two general methods are available for immunoassay detection; direct and indirect assays. According to the first assay, binding of antibody to the target antigen is determined directly. This direct assay uses a labeled reagent, such as a fluorescent tag or an enzyme-labeled primary antibody, which can be visualized without further antibody interaction. In a typical indirect assay, unconjugated primary antibody binds to the antigen and then a labeled secondary antibody binds to the primary antibody. Where the secondary antibody is conjugated to an enzymatic label, a chromogenic or fluorogenic substrate is added to provide visualization of the antigen. Signal amplification occurs because several secondary antibodies may react with different epitopes on the primary antibody.

The primary and/or secondary antibody typically will be labeled with a detectable moiety. Numerous labels are available which can be generally grouped into the following categories:
(a) Radioisotopes, such as ³⁵S, ¹⁴C, ¹²⁵I, ³H, and ¹³¹I. The antibody can be labeled with the radioisotope using the techniques described in Current Protocols in Immunology, Volumes 1 and 2, Coligen et al., Ed. Wiley-Interscience, New York, N.Y., Pubs. (1991) for example and radioactivity can be measured using scintillation counting.
(b) Colloidal gold particles.
(c) Fluorescent labels including, but are not limited to, rare earth chelates (europium chelates), Texas Red, rhodamine, fluorescein, dansyl, Lissamine, umbelliferone, phycocrytherin, phycocyanin, or commercially available fluorophores such SPECTRUM ORANGE7 and SPECTRUM GREEN7 and/or derivatives of any one or more of the above. The fluorescent labels can be conjugated to the antibody using the techniques disclosed in Current Protocols in Immunology, supra, for example. Fluorescence can be quantified using a fluorimeter.
(d) Various enzyme-substrate labels are available and U.S. Pat. No. 4,275,149 provides a review of some of these. The enzyme generally catalyzes a chemical alteration of the chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for use in Enzyme Immunoassay, in Methods in Enzym. (ed. J. Langone & H. Van Vunakis), Academic press, New York, 73:147-166 (1981).

Examples of enzyme-substrate combinations include, for example:
(i) Horseradish peroxidase (HRPO) with hydrogen peroxidase as a substrate, wherein the hydrogen peroxidase oxidizes a dye precursor (e.g., orthophenylene diamine (OPD) or 3,3′,5,5′-tetramethyl benzidine hydrochloride (TMB));
(ii) alkaline phosphatase (AP) with para-Nitrophenyl phosphate as chromogenic substrate; and
(iii) β-D-galactosidase (β-D-Gal) with a chromogenic substrate (e.g., p-nitrophenyl-β-D-galactosidase) or fluorogenic substrate (e.g., 4-methylumbelliferyl-β-D-galactosidase).

Numerous other enzyme-substrate combinations are available to those skilled in the art. For a general review of these, see U.S. Pat. Nos. 4,275,149 and 4,318,980. Sometimes, the label is indirectly conjugated with the antibody. The skilled artisan will be aware of various techniques for achieving this. For example, the antibody can be conjugated with biotin and any of the four broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten and one of the different types of labels mentioned above is conjugated with an anti-hapten antibody. Thus, indirect conjugation of the label with the antibody can be achieved.

Following an optional blocking step, the sample is exposed to primary antibody for a sufficient period of time and under suitable conditions such that the primary antibody binds to the target protein antigen in the sample. Appropriate conditions for achieving this can be determined by routine experimentation. The extent of binding of antibody to the sample is determined by using any one of the detectable labels discussed above. In certain embodiments, the label is an enzymatic label (e.g. HRPO) which catalyzes a chemical alteration of the chromogenic substrate such as 3,3′-diaminobenzidine chromogen. In one embodiment, the enzymatic label is conjugated to antibody which binds specifically to the primary antibody (e.g. the primary antibody is rabbit polyclonal antibody and secondary antibody is goat anti-rabbit antibody).

In some embodiments, the sample may be contacted with an antibody specific for said biomarker under conditions sufficient for an antibody-biomarker complex to form, and then detecting said complex. The presence of the biomarker may be detected in a number of ways, such as by Western blotting and ELISA procedures for assaying a wide variety of tissues and samples, including plasma or serum. A wide range of immunoassay techniques using such an assay format are available, see, e.g., U.S. Pat. Nos. 4,016,043, 4,424,279 and 4,018,653. These include both single-site and two-site or “sandwich” assays of the non-competitive types, as well as in the traditional competitive binding assays. These assays also include direct binding of a labeled antibody to a target biomarker.

Sandwich assays are among the most useful and commonly used assays. A number of variations of the sandwich assay technique exist, and all are intended to be encompassed by the present invention. Briefly, in a typical forward assay, an unlabeled antibody is immobilized on a solid substrate, and the sample to be tested brought into contact with the bound molecule. After a suitable period of incubation, for a period of time sufficient to allow formation of an antibody-antigen complex, a second antibody specific to the antigen, labeled with a reporter molecule capable of producing a detectable signal is then added and incubated, allowing time sufficient for the formation of another complex of antibody-antigen-labeled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of biomarker.

Variations on the forward assay include a simultaneous assay, in which both sample and labeled antibody are added simultaneously to the bound antibody. These techniques are known to those skilled in the art, including any minor variations as will be readily apparent. In a typical forward sandwich assay, a first antibody having specificity for the biomarker is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g. 2-40 minutes or overnight if more convenient) and under suitable conditions (e.g. from room temperature to 40° C. such as between 25° C. and 32° C. inclusive) to allow binding of any subunit present in the antibody. Following the incubation period, the antibody subunit solid phase is washed and dried and incubated with a second antibody specific for a portion of the biomarker. The second antibody is linked to a reporter molecule which is used to indicate the binding of the second antibody to the molecular marker.

An alternative method involves immobilizing the target biomarkers in the sample and then exposing the immobilized target to specific antibody which may or may not be labeled with a reporter molecule. Depending on the amount of target and the strength of the reporter molecule signal, a bound target may be detectable by direct labelling with the antibody. Alternatively, a second labeled antibody, specific to the first antibody is exposed to the target-first antibody complex to form a target-first antibody-second antibody tertiary complex. The complex is detected by the signal emitted by the reporter molecule. By “reporter molecule”, as used in the present specification, is meant a molecule which, by its chemical nature, provides an analytically identifiable signal which allows the detection of antigen-bound antibody. The most commonly used reporter molecules in this type of assay are either enzymes, fluorophores or radionuclide containing molecules (i.e. radioisotopes) and chemiluminescent molecules.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, -galactosidase and alkaline phosphatase, amongst others. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above. In all cases, the enzyme-labeled antibody is added to the first antibody-molecular marker complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of biomarker which was present in the sample. Alternately, fluorescent compounds, such as fluorescein and rhodamine, may be chemically coupled to antibodies without altering their binding capacity. When activated by illumination with light of a particular wavelength, the fluorochrome-labeled antibody adsorbs the light energy, inducing a state to excitability in the molecule, followed by emission of the light at a characteristic color visually detectable with a light microscope. As in the EIA, the fluorescent labeled antibody is allowed to bind to the first antibody-molecular marker complex. After washing off the unbound reagent, the remaining tertiary complex is then exposed to the light of the appropriate wavelength, the fluorescence observed indicates the presence of the molecular marker of interest. Immunofluorescence and EIA techniques are both very well established in the art. However, other reporter molecules, such as radioisotope, chemiluminescent or bioluminescent molecules, may also be employed.

The IL-13 status of a patient based on the test results (e.g., elevated, above the reference, or below the reference) may be provided in a report. The report may be in any form of written materials (e.g., in paper or digital form, or on internet) or oral presentation(s) (e.g., either in person (live) or as recorded). The report may further indicates to a health professional (e.g., a physician) that the patient may benefit from or is likely to respond to an interferon inhibitor treatment.

The kits of the invention have a number of embodiments. In certain embodiments, a kit comprises a container, a label on said container, and a composition contained within said container; wherein the composition includes one or more primary antibodies that bind to one or more target polypeptide sequences corresponding to one or more biomarkers, including IL-13, the label on the container indicating that the composition can be used to evaluate the presence of one or more target proteins in at least one type of mammalian cell, and instructions for using the antibodies for evaluating the presence of one or more target proteins in at least one type of mammalian cell. The kit can further comprise a set of instructions and materials for preparing a tissue sample and applying antibody and probe to the same section of a tissue sample. The kit may include both a primary and secondary antibody, wherein the secondary antibody is conjugated to a label, e.g., an enzymatic label.

The term “detecting” encompasses quantitative or qualitative detection. In certain embodiments, a biological sample comprises a cell or tissue, such as serum, plasma, nasal swabs and sputum.

### Pharmaceutical Formulations

Pharmaceutical formulations of an anti-IL-13 antibody or other Th2 pathway inhibitors as described herein are prepared by mixing such antibody or molecule having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. For example, it may be desirable to further provide a controller with the Th2 pathway inhibitor. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

### Therapeutic Methods and Compositions

Eosinophilic inflammation is associated with a variety of illnesses, both allergic and non-allergic (Gonlugur (2006) Immunol. Invest. 35(1):29-45). Inflammation is a restorative response of living tissues to injury. A characteristic of inflammatory reactions is the accumulation of leukocytes in injured tissue due to certain chemicals produced in the tissue itself. Eosinophil leukocytes accumulate in a wide variety of conditions such as allergic disorders, helminthic infections, and neoplastic diseases (Kudlacz et al., (2002) Inflammation 26: 111-119). Eosinophil leukocytes, a component of the immune system, are defensive elements of mucosal surfaces. They respond not only to antigens but to parasites, chemicals, and trauma.

Tissue eosinophilia occurs in skin diseases such as eczema, pemphigus, acute urticaria, and toxic epidermal necrolysis as well as in atopic dermatitis (Rzany et al., Br. J. Dermatol. 135: 6-11 (1996)). Eosinophils accumulate in the tissue and empty granule proteins in IgE-mediated allergic skin reactions (Nielsen et al., Ann. Allergy Asthma Immunol., 85: 489-494 (2001)). Eosinophils combined with mast cells are likely to cause joint inflammation (Miossec, J. Clin. Rheumatol. 3: 81-83 (1997)). Eosinophilic inflammation sometimes accompanies joint trauma. Synovial fluid eosinophilia can be associated with diseases such as rheumatoid arthritis, parasitic disease, hypereosinophilic syndrome, Lyme disease, and allergic processes, as well as hemarthrosis and arthrography (Atanes et al., Scand. J. Rheumatol., 25: 183-185 (1996)). Eosinophilic inflammation can affect bones as well (Yetiser et al., Int. J. Pediatr. Otorhinolaryngol., 62: 169-173 (2002)). Examples of eosinophilic muscle disease include eosinophilic perimyositis, eosinophilic polymyositis, and focal eosinophilic myositis (Lakhanpal et al., Semin. Arthritis Rheum., 17: 331-231 (1988)). Eosinophilic inflammations affecting skeletal muscles may be associated with parasite infections or drugs or features of some systemic disorders of hypereosinophilia (e.g., idiopathic hypereosinophilic syndrome and eosinophilia-myalgia syndrome. Eosinophils participate in the inflammatory response to epitopes recognized by autoimmune antibodies (Engineer et al., Cytokine, 13: 32-38 (2001)). Connective tissue diseases may lead to neutrophilic, eosinophilic, or lymphocytic vascular inflammations (Chen et al., J. Am. Acad. Dermatol., 35: 173-182 (1996)). Tissue and peripheral blood eosinophilia can occur in active rheumatismal diseases. Elevation of serum ECP levels in ankylosing spondylitis, a kind of connective tissue disease, suggests that eosinophils are also involved in the underlying process (Feltelius et al., Ann. Rheum. Dis., 46: 403-407 (1987)). Wegener's granulomatosis can rarely present with pulmonary nodules, pleural effusion, and peripheral blood eosinophilia (Krupsky et al., Chest, 104: 1290-1292 (1993)).

Peripheral blood eosinophilia of at least 400/mm3 can occur in 7% of cases of systemic sclerosis, 31% of cases of localized scleroderma, and 61% of cases of eosinophilic fasciitis (Falanga, et al., J. Am. Acad. Dermatol., 17: 648-656 (1987)). Scleroderma yields an inflammatory process closely resembling Meissner's and Auerbach's plexuses and consists of mast cells and eosinophil leukocytes in the gastrointestinal system. Eosinophil-derived neurotoxins can contribute to gastrointestinal motor dysfunction, as occurs in scleroderma (DeSchryver-Kecskemeti, et al. Arch. Pathol. Lab Med., 113: 394-398 (1989)).

Eosinophils can accompany localized (Varga, et al., Curr. Opin. Rheumatol., 9: 562-570 (1997)) or systemic (Bouros et al., Am. J. Respir. Crit. Care Med., 165: 1581-1586 (2002)) connective tissue proliferation. They can incite fibrosis by inhibiting proteoglycan degradation in fibroblasts (Hemnas et al., Eur. J. Cell Biol., 59: 352-363 (1992)), and fibroblasts mediate eosinophil survival by secreting GM-CSF (Vancheri et al., Am. J. Respir. Cell Mol. Biol., 1: 289-214 (1989)). Eosinophils can be found in nasal (Bacherct et al., J. allergy Clin. Immunol., 107: 607-614 (2001)), bronchial (Arguelles, et al., Arch. Intern. Med., 143: 570-571 (1983)), and gastrointestinal polyp tissues (Assarian, et al., Hum. Pathol., 16: 311-312 (1985)). Likewise, eosinophils can be localized in inflammatory pseudotumors (myofibroblastic tumor). Eosinophils often accompany inflammatory pseudotumors in the orbital region, in which case the condition can mimic angioedema or allergic rhinoconjunctivitis (Li et al., Ann. Allergy, 69: 101-105 (1992)).

Eosinophilic inflammation can be found in tissue trauma (e.g., as a result of surgery or injury). Eosinophilic inflammation can also be associated with cardiovascular illnesses (e.g., eosinophilic myocarditis, eosinophilic coronary arteritis, ischemic heart disease, acute myocardial infarction, cardiac rupture). Necrotic inflammatory processes can also involve eosinophililic inflammation (polymyositis, coronary artery dissection, necrotizing lesions of neuro-Behcet's disease, dementia, cerebral infarction).

Among noninvasive biomarkers of the Th2-driven/eosinophilic asthma subphenotype are serum periostin, fractional exhaled nitric oxide (FeNO), and peripheral blood eosinophil count. See Arron et al. (2013) Adv Pharmacol 66: 1-49. Of these markers, serum periostin has been advanced as a predictive diagnostic for lebrikizumab because it was the best single predictor of airway eosinophil status (as determined by a composite of sputum and tissue eosinophilia) in the BOBCAT observation study of severe asthma (Jia et al. (2012) J Allergy Clin Immunol 130: 647-654 e10), it exhibited substantially less intra-patient variability than FeNO or blood eosinophils across two pre-dose visits in the MILLY study (Corren et al. (2011) N Engl J Med 365: 1088-98), and can be available on a standardized, broadly available assay platform that requires neither a specialized point-of-care instrument (such as FeNO), nor is dependent on automated cell counters that are not broadly standardized across existing clinical laboratories (such as blood eosinophils). While serum periostin appears to be a robust and consistent biomarker for the Th2/eosinophilic subtype of adult asthma, whether it can be applied to pediatric asthma was unknown.

In the MILLY study, adults with poorly controlled asthma despite ICS who had serum periostin levels above 50 ng/ml at baseline exhibited a mean 14.4% reduction in serum periostin after 12 weeks of lebrikizumab treatment (p=0.001), while patients with baseline serum periostin levels below 50 ng/ml exhibited a non-significant 2.9% reduction in serum periostin during the treatment period (p=0.3). See Scheerens et al. (2012) Am J Respir Crit Care Med 185: A3960. The distribution of serum periostin levels in asthma patients after 12 weeks of lebrikizumab treatment overlapped with the distribution of serum periostin levels in healthy control adults (Arron et al, Annals Am. Thoracic Soc., in press (2013), DOI: 10.1513/AnnalsATS.201303-047AW). These results suggest that, in adult asthmatic patients with high serum periostin, the excess periostin above background levels is due to the activity of IL13 in the airways, and this excess constitutes about 10-15% of total systemic periostin.

Periostin was initially identified as a product of osteoblasts, the cells that lay down bone matrix. See Horiuchi et al. (1999) J Bone Miner Res 14: 1239-49. Anatomically, periostin expression in bone is localized to sites of endochondral and intramembranous ossification during development, suggesting that periostin expression levels may be correlated with the rate of bone growth. In juvenile mice, systemic periostin levels and markers of bone turnover are elevated, decreasing as animals mature and attaining relatively stable levels from the age of 8 weeks throughout adulthood. See Contie et al. (2010) Calcif Tissue Int 87: 341-5. In humans, while asthma in the pediatric population is more commonly associated with atopy and type 2 inflammation than in adults, there remains evidence for eosinophilic and non-eosinophilic airway inflammatory subsets in asthmatic children. See Baraldo et al. (2011) Eur Respir J 38: 575-83. Hence biomarkers that identify asthmatic children with increased Th2/eosinophilic airway inflammation may be useful to enable patient selection to demonstrate clinical benefit from anti-IL13 and other therapeutics targeting type 2 inflammation.

Provided herein are methods of identifying patients having elevated circulating IL-13 levels which is predictive for a response to treatment with a Th2 pathway inhibitor (or that will be responsive to) by measuring levels of IL-13 in a biological sample from a patient using the IMPACT IL-13 assay described herein.

Also provided herein are methods of treating asthma, a Th2-associated disease, an IL-13 mediated Disorder, an IL4 mediated Disorder, an IL9 mediated Disorder, an IL5 mediated Disorder, an IL33 mediated Disorder, an IL25 mediated Disorder, an TSLP mediated Disorder, an IgE-mediated Disorder or Asthma-Like Symptoms comprising administering a Th2 pathway inhibitor to a patient having elevated circulating IL-13 levels, wherein the patient was diagnosed using an IMPACT IL-13 assay as described herein.

Also provided are methods of treating asthma comprising administering a therapeutically effective amount of lebrikizumab to the asthma patient, wherein the treatment results in a relative change in FEV₁ of greater than 5%. In another embodiment, the FEV₁ is greater than 6%, 7%, 8%, 9% or 10% FEV₁. In another embodiment, the patient has been diagnosed as having elevated circulating IL-13 using IMPACT IL-13 assay.

In certain embodiments, methods of treating asthma comprising administering a therapeutically effective amount of lebrikizumab to the asthma patient, wherein the treatment results in a reduction in exacerbation rate of greater than 35%. (other embodiments greater than 36%, 37%, 38%, 39%, 40%, 41%, 42%, 43%, 44%, 45%, 46%, 47%, 48%, 49%, 50%, 51%, up to 85%; another embodiment, wherein the patient has been diagnosed as having elevated circulating IL-13 levels) are provided.

In certain embodiments, methods of treating asthma comprising administering a therapeutically effective amount of lebrikizumab to the asthma patient, wherein the treatment results in a reduction in nocturnal awakenings are provided. In one embodiment, the patient is diagnosed as having elevated circulating IL-13 levels using IMPACT IL-13 assay. In another embodiment, the asthma of the patient is uncontrolled on a corticosteroid. In another embodiment, the patient is diagnosed as having elevated circulating IL-13 levels.

Also provided are methods of treating asthma comprising administering a therapeutically effective amount of lebrikizumab to the asthma patient, wherein the treatment results in an improvement in asthma control In one embodiment, the patient is diagnosed as having elevated circulating IL-13 levels using IMPACT IL-13 assay. In another embodiment, the asthma of the patient is uncontrolled on a corticosteroid. In another embodiment, the patient is diagnosed as having elevated circulating IL-13 levels.

Methods of treating asthma (or Respiratory Disease) comprising administering a therapeutically effective amount of lebrikizumab to the asthma patient, wherein the treatment results in a reduction of inflammation in the lungs are provided. In one embodiment, the patient is diagnosed as having elevated circulating IL-13 levels using IMPACT IL-13 assay. In another embodiment, the asthma of the patient is uncontrolled on a corticosteroid. In another embodiment, the patient is diagnosed as having elevated circulating IL-13 levels.

In certain embodiments, methods of treating Th2-associated disorder in a patient suffering from the Th2-associated disorder and being treated with a corticosteroid comprising administering a therapeutically effective amount of lebrikizumab to the patient, wherein the treatment results in a reduction or elimination of corticosteroid treatment (amount or frequency) used to treat the disease are provided. In one embodiment, the patient is diagnosed as having elevated circulating IL-13 levels using IMPACT IL-13 assay. In another embodiment, the asthma of the patient is uncontrolled on a corticosteroid. In another embodiment, the patient is diagnosed as having elevated circulating IL-13 levels.

Also provided are methods of treating of a patient suffering from asthma (or Th2-associated disease) comprising diagnosing the patient as having elevated IL-13 levels using IMPACT IL-13 assay, administering a therapeutically effective amount of Th2 pathway inhibitor to the asthma patient, diagnosing the patients IL-13 status, and retreating the patient with the Th2 pathway inhibitor if the IL-13 status is elevated or above the reference level. The diagnosis may be made using an immunoassay (e.g., IMPACT IL-13) alone or in combination with FE_{NO} levels, periostin levels, blood eosinophil levels, or IgE.

Any of the Th2 pathway inhibitors provided herein may be used in therapeutic methods described herein, especially asthma. In one embodiment, the asthma patient is being treated with a corticosteroid, and has been diagnosed as responsive a Th2 pathway inhibitor using an immunoassay described herein. In a further embodiment, the asthma patient is suffering from moderate to severe asthma. In another embodiment, the patient is suffering from mild asthma but is not being treated with a corticosteroid.

An antibody of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the invention (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 15 mg/kg (e.g. 0.1 mg/kg-10 mg/kg) of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). However, other dosage regimens may be useful. The progress of this therapy is easily monitored by conventional techniques and assays.

In certain embodiments, an antibody of the invention is administered as a flat dose (i.e., not weight dependent) of 37.5 mg, or a flat dose of 125 mg, or a flat dose of 250 mg. In certain embodiments, the dose is administered by subcutaneous injection once every 4 weeks for a period of time. In certain embodiments, the period of time is 6 months, one year, two years, five years, ten years, 15 years, 20 years, or the lifetime of the patient. In certain embodiments, the asthma is severe asthma and the patient is inadequately controlled or uncontrolled on inhaled corticosteroids plus a second controller medication.

It is understood that any of the above formulations or therapeutic methods may be carried out using an immunoconjugate of the invention in place of or in addition to an anti-target antibody.

### Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

It is understood that any of the above articles of manufacture may include an immunoconjugate in place of or in addition to an anti-target antibody.

### Examples

### Example 1—Immunoassay Methods

### Human Serum Samples:

For the experiments described in Examples 1-3, we used serum samples from healthy volunteers (HV) to characterize the commercially available Erenna® IL-13 Immunoassay or the IMPACT IL-13 assay, as indicated below. These healthy volunteer samples were obtained from an internal blood donation program (Genentech, Inc., South San Francisco, Calif., USA; Roche Professional Diagnostics, Penzberg, Germany). In addition, as indicated below, serum samples from patients with asthma, IPF or atopic dermatitis were purchased from BioreclamationIVT (New York, USA).

The Immunological Multiparameter Chip Technology (IMPACT) platform has been described previously. See, e.g., WO 2007/039280 and Claudon et al., Clinical Chemistry 54(9):1554-1563. Below are a brief general description of the platform and the general procedure followed by a description of the IMPACT IL-13 assay.

The IMPACT technology is based on a black polystyrene chip with a surface area of about 2.5×6 mm manufactured using streptavidin-biotin interactions. The chip surface is coated with a streptavidin layer, onto which a first biotinylated capture antibody (which specifically binds to an analyte) is spotted using ink-jet technology. Each spot is about 150 μM in diameter. During the assay, the chip is incubated with specimen samples containing the analyte and a second digoxigenylated monoclonal antibody of different epitopic specificity from the first antibody and that specifically binds the analyte. The third detection antibody is an anti-digoxigenen monoclonal antibody coupled with fluorescent latex conjugate. It was previously reported (Claudon et al., Clinical Chemistry 54(9):1554-1563) that using this label, less than ten individual binding events in a single spot can be detected, resulting in very high sensitivity. Chips are transported into a detection unit and a charge coupled device (CCD) camera generates an image that is transformed into signal intensities using dedicated software. Individual spots are automatically located at predefined positions and quantified by image analysis. Analytes that have been successfully detected and quantitated using the IMPACT platform include anti-CCP antibodies, antinuclear antibodies, serum C-terminal cross-linked telopeptide of type I collagen, N-terminal propeptide of type I collagen, osteocalcin, and intact parathyroid hormone. The reported sensitivities (reported as LLOQ) for these analytes when run as single assays range from 0.087 μg/L (0.087 ng/mL) to 4.9 ng/L (4.9 pg/mL) (Id.).

We used two different proprietary anti-IL-13 antibodies as a capture reagent and a detection reagent, respectively, for the IMPACT IL-13 assay. The first anti-IL-13 antibody (the “capture” antibody) was lebrikizumab (also referred to as MILR1444A) which has been described previously. See, e.g., WO 2005/062967 and Ultsch et al., J. Mol. Biol. 425:1330-9 (2013). The second anti-IL-13 antibody (the “detection” antibody) was 11H4 which has also been described previously, see, e.g., WO 2014/165771. The F(ab′)₂ of lebrikizumab was prepared and biotinylated using standard procedures that are well known in the art. The biotinylated lebrikizumab F(ab′)₂ was spotted onto streptavidin-coated polystyrene chips as described above, each chip with 60 identical spots of 160 μM diameter. To initiate the assay, samples were diluted with Sample Buffer (Sample Buffer (pH 7.25) comprised 75 mM dipotassium hydrogen phosphate, 25 mM potassium dihydrogen phosphate, 150 mM NaCl, 0.01% methylisothiazolone, 0.01% Bronidox®, 0.16% Tween®20, 0.08% polydocanol, 2 mM EDTA, 2% BSA, 1% bovine IgG, 5% horse serum (Sigma, Cat. No. H1470). Twelve μL of sample was diluted with 28 μL of Sample Buffer and incubated with the chips for 18 minutes at 36° C. The reaction was terminated by washing the chip with 0.8 mL Wash Buffer for 5 seconds (Wash Buffer composition was as follows: 10 mM Tris-C1, pH 8.0, 0.001% methylisothiazol-hydrochloride (MIT), 0.001% Oxy-Pyrion, 0.01% polydocanol). The second anti-IL-13 antibody, full-length 11H4 (IgG1), was labeled with digoxigenin using standard methods that are well known in the art. For detection, digoxigenylated anti-IL-13 11H4 antibody and the anti-digoxigenin monoclonal antibody conjugated with fluorescent-tag labeled latex were added sequentially. The reaction was terminated by washing the chip with 2.0 mL of Wash Buffer for 10 seconds followed by drying for 10 seconds using suction. Fluorescence intensity was detected using a CCD-detector and quantified using proprietary software as described above. Recombinant human IL-13 (R&D Systems, MN, catalog no. 213-ILB/CF) was used to generate a standard curve ranging from 2-1000 pg/ml; dilutions of recombinant human IL-13 were made in the following buffer: 20 mM potassium phosphate pH 7.35, 50 mM NaCl, 4.0% sucrose, 2.0% BSA, 0.2% bovine IgG, 0.01% MIT, 0.01% Bronidox®, 0.2% Tween®-20.

A commercially available IL-13 assay for detecting and quantifying IL-13 levels in human serum and plasma was also used. The commercially available IL-13 assay was Erenna® IL-13 Immunoassay Kit from Singulex® (Alameda, Calif.) (Version 1, Cat#03-0069-00, is no longer available; version 2, Cat#03-0109-xx, which is commercially available, was not used in the experiments described herein and has a reported LLOQ of 0.04 pg/mL [Erenna® IL-13 (v2) Immunoassay Kit, Cat. #03-0109-xx Product Information Sheet, available at www(dot)singulex(dot)com)]). Except where noted, the Erenna® IL-13 Immunoassay, Version 1, was performed in accordance with the manufacturer's instructions.

### Example 2—Commercially Available IL-13 Assay and Optimization Efforts

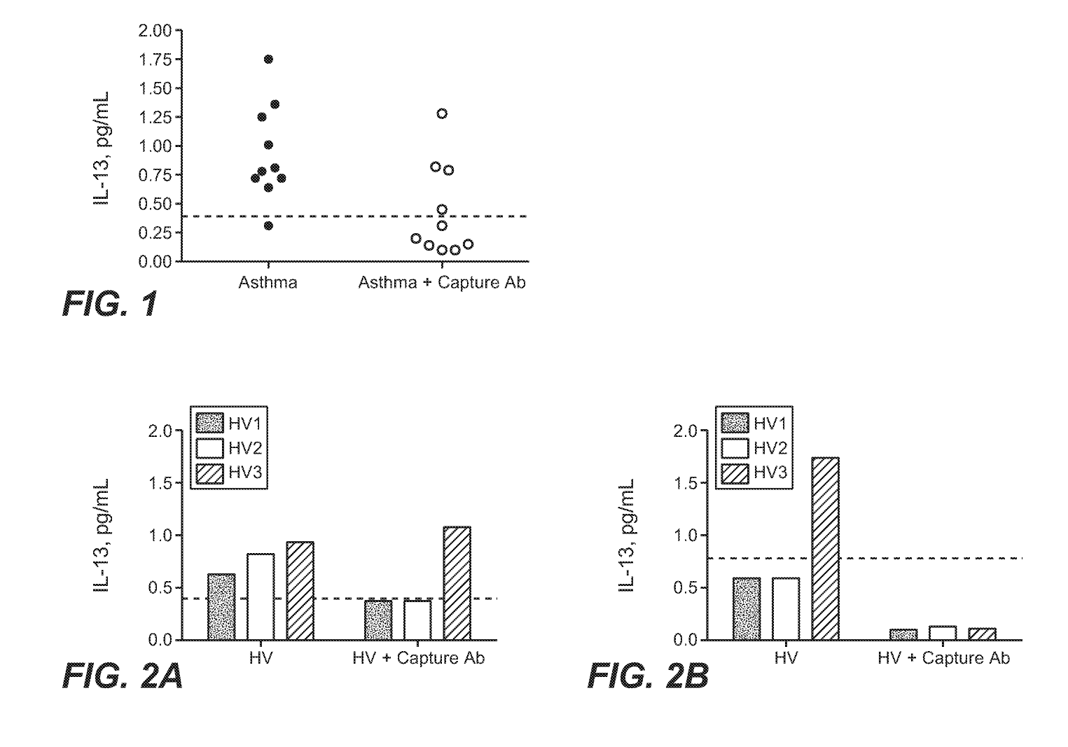

Using the manufacturer's recommended assay conditions, we tested ten serum samples from asthmatic patients. Nine out of those 10 samples (90%) had detectable levels of IL-13 above the LLOQ of 0.39 pg/mL, the LLOQ specified by the manufacturer. The quantified IL-13 levels ranged from 0.64 pg/mL to 1.75 pg/mL (FIG. 1). Assay specificity for IL-13 was tested by pre-incubating the samples with excess capture antibody. The pre-incubation was carried out for one hour at room temperature. This is referred to as a competition method or an immunodepletion method to assess specificity. The assay signal from six of the asthmatic serum samples was effectively competed to levels below the LLOQ using this approach (FIG. 1). But the assay signal from four of the asthmatic serum samples with higher IL-13 levels was not effectively competed indicating a lack of specificity. The apparently non-specific signal in these samples appeared significant, generating a signal that ranged from 0.45 pg/mL to 1.28 pg/mL (FIG. 1).

The results of the specificity assessment discussed above suggested that the specificity of the Erenna® IL-13 Immunoassay was non-optimal. Therefore, we explored whether modification of the manufacturer's recommended assay conditions for sample diluent and minimum required dilution would improve specificity. Serum samples from three HVs were assayed according to the manufacturer's recommendations (neat) or alternatively, were diluted 1:1 (V/V) with the manufacturer's high salt buffer. FIG. 2A shows that, following the manufacturer's recommendations, IL-13 in each of the HV samples was above the LLOQ, however, the HV sample containing the highest level of IL-13 could not be competed by pre-incubation with excess capture antibody coated on microparticle beads (25 μg capture antibody/mg microparticle beads). Thus, this result was consistent with the prior result obtained using the ten asthmatic serum samples (compare FIG. 2A to FIG. 1). In contrast, dilution of each of the HV samples 1:1 (V/V) with high salt buffer demonstrated effective competition by pre-incubation with excess capture antibody coated on microparticle beads (25 μg capture antibody/mg microparticle beads) because the detected assay signal was below the modified assay LLOQ of 0.78 pg/mL (FIG. 2B, right side), suggesting that high salt did increase the assay specificity. Note that the LLOQ was modified from 0.39 pg/mL to 0.78 pg/mL to account for the two-fold dilution of the sample. However, only one of the HV samples showed a signal above the modified assay LLOQ, suggesting that the improved specificity was obtained at the expense of sensitivity (FIG. 2B, left side).

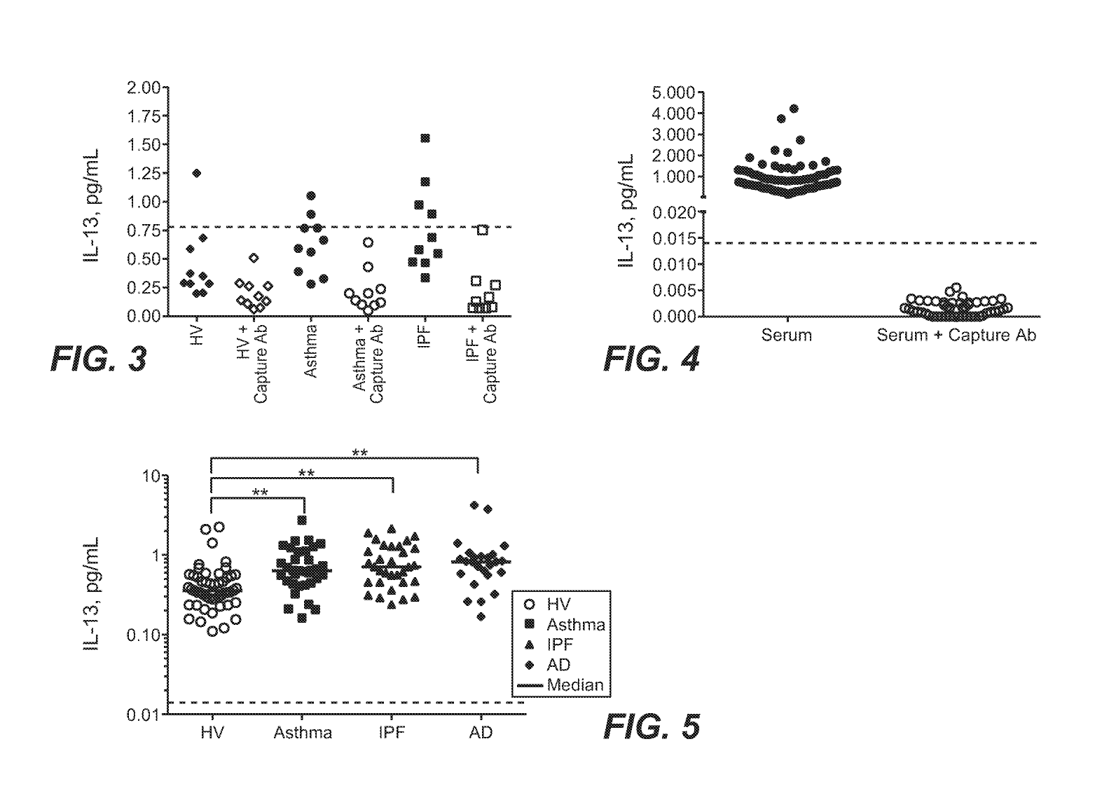

We next measured the IL-13 levels in serum samples from asthma (n=10) and IPF patients (n=10), and HV (n=10) using the modified assay conditions. IL-13 levels were detectable in only 40% (n=10) of the asthma patient samples, 40% (n=10) of the IPF patient samples and 10% (n=10) of the HV samples (FIG. 3). The specificity of the assay signal for IL-13 was confirmed by pre-incubation with excess capture antibody coated on microparticle beads (25 μg capture antibody/mg microparticle beads) prior to testing in the assay. The IL-13 levels observed were between 0.78 pg/mL to 1.05 pg/mL in the asthma patient samples, between 0.89 pg/mL to 1.56 pg/mL in the IPF patient samples; only one HV sample had detectable levels of IL-13, 1.25 pg/mL (FIG. 3).

Based on the above-described results, we concluded that the Erenna® IL-13 Immunoassay was not sufficiently sensitive and specific to allow for detection and accurate quantification of serum IL-13 levels in a majority of patients suffering with a variety of Th-2 associated diseases. As discussed above, there is a need for a highly sensitive and highly specific serum IL-13 assay so that serum IL-13 levels can be accurately quantitated in healthy individuals and various Th-2 associated disease states. Accurate quantitation of serum IL-13 levels would allow for comparisons between healthy individuals and various Th-2 associated disease states which can provide insights into disease mechanisms and progression, as well as identify patients who may most benefit from therapeutic interventions targeting the IL-13 pathway or the Th2 pathway. Accurate quantiation of serum IL-13 levels would also facilitate investigation of the pharmoacodynamic effects of certain therapeutics that target IL-13 as well as certain therapeutics against other targets in the Th2 pathway.

### Example 3—Impact IL-13 Assay Development and Characterization

To develop a highly sensitive and highly specific serum IL-13 assay, we began by testing pair-wise combinations of eight different IL-13 antibodies, some commercially available and some generated by Genentech. Table 2 below lists the antibody pairs. For each of the 56 paired combinations, we determined the negative (blank) signal and the positive (specific) signal and the ratio of positive signal/negative signal using 100 pg/mL recombinant IL-13 in Sample Buffer (see Example 1 for Sample Buffer composition). The antibody pair combination providing the best positive signal/negative signal ratio (i.e. the highest positive signal [“Pos”] and the lowest negative signal [“Neg”] or a signal to noise ratio) is identified by gray boxes in Table 2 and was selected for further assay optimization. Specifically, this optimal antibody pair combination was MILR1444A as the capture antibody and 11H4 as the detection antibody.

TABLE 2
Negative and positive assay signals for anti-IL-13 antibody pairs

| Second | First Capture Antibody^{a} | | | | | | | |
|---|---|---|---|---|---|---|---|---|
| Detection | Assay | | | | | | | |
| Antibody^{a} | Signal | MILR1444A | 228B/C | 11H4 | 14C9^{b} | 4D7^{c} | 8C11^{d} | 32116^{e} |
| MILR1444A | Neg | 6.4 | 28.5 | 22.5 | 33.7 | 22.0 | 4.2 | 13.6 |
| | Pos | 6.5 | 27.3 | 8528.6 | 85.1 | 25.0 | 17.3 | 78.6 |
| 228B/C | Neg | 5.4 | 26.6 | 22.4 | 29.0 | 24.7 | 3.9 | 15.2 |
| | Pos | 6.0 | 27.7 | 9543.5 | 70.3 | 29.9 | 25.4 | 125.6 |
| 11H4 | Neg | 0.4 | 26.5 | 23.3 | 29.0 | 21.3 | 3.5 | 13.7 |
| | Pos | 9101.2 | 8397.2 | 396.7 | 36.8 | 23.2 | 792.9 | 822.9 |
| 14C9 | Neg | 1.3 | 27.8 | 23.8 | 30.5 | 23.6 | 4.6 | 15.8 |
| | Pos | 345.7 | 354.3 | 55.9 | 31.7 | 23.0 | 25.1 | 29.3 |
| 4D7 | Neg | 4.7 | 42.1 | 22.9 | 29.6 | 28.4 | 4.2 | 16.4 |
| | Pos | 45.9 | 90.5 | 32.1 | 31.9 | 27.6 | 39.2 | 19.4 |
| 8C11 | Neg | 1.8 | 24.5 | 22.1 | 27.8 | 20.9 | 8.0 | 12.9 |
| | Pos | 40.9 | 70.8 | 6738.9 | 109.7 | 53.8 | 9.5 | 14.6 |
| MAB213^{b} | Neg | 0.3 | 26.5 | 23.2 | 30.0 | 21.7 | 3.9 | 17.2 |
| | Pos | 47.2 | 52.7 | 2311.2 | 33.2 | 23.6 | 4.1 | 18.2 |
| Polyclonal | Neg | 30.4 | 24.0 | 19.1 | 28.2 | 21.1 | 6.8 | 18.5 |
| AF213- | Pos | 6583.0 | 6343.0 | 168.2 | 1388.1 | 58.6 | 1914.7 | 53.6 |
| NA^{c} | | | | | | | | |

^{a}All antibodies are proprietary Genentech antibodies unless indicated otherwise. All antibodies are murine antibodies, other than MILR1444A, which is a humanized mouse antibody.
^{b}R&D Systems, MN, Cat. No. MAB213
^{c}R&D Systems, MN, Cat. No. AF213-NA

As shown in Table 2, the results with 228B/C as the capture antibody were different from the results with MILR1444A as the capture antibody. The MILR1444A-11H4 positive/negative ratio was 22,753 and the 228B/C-11H4 positive/negative ratio was 316.87. That these results were different was surprising because MILR1444A is a humanized variant of 228B/C. These two antibodies not only have the same CDRs but also a similar affinity for IL-13 (see, e.g., WO 2005/062967), thus indicating that capture antibody affinity for antigen and/or epitope are not the sole contributors to the assay positive/negative ratio. In addition, we observed that the detection antibody influenced the positive/negative ratio when the capture antibody was kept constant. Using MILR1444A as the capture antibody and different detection antibodies that bind different epitopes with different affinities (i.e., 14C9, 4D7, 8C11, MAB213 and AF-213-NA) yielded a wide range of positive/negative ratios (Table 2). In summary, the pairwise analysis of capture and detection antibodies presented in Table 2 shows that the optimal combination of capture antibody and detection antibody could not have been predicted from information known about the antibodies, e.g., epitope, affinity, prior to carrying out the experiments.

We used Sample Buffer (see Example 1 for Sample Buffer composition) and Detection Buffer (composition (pH 8.5) comprising 80 mM TAPS, 500 mM sodium chloride, 0.01% methyl iso thioazolone, 0.01% Bronidox®, 0.08% Tween®20, 0.04% polydocanol, 0.3% BSA, 0.25% bovine IgG and 0.1% casein) in the following experiments. The optimal anti-IL-13 antibody pair was determined to be lebrikizumab (also referred to as MILR1444A), which in some embodiments is F(ab′)₂ and in some embodiments is Fab, for the first (capture) antibody and 11H4 for the second (detection) antibody as described above.

After the optimal anti-IL-13 antibody pair had been selected, we proceeded to determine intra-assay precision, inter-assay precision, LLOQ, accuracy and linearity/parallelism, and interference from soluble IL-13Rα2 as described below. For these assessments, we used serum samples from healthy volunteers to characterize the IMPACT IL-13 assay. These healthy volunteer samples were obtained from an internal blood donation program (Genentech, Inc., South San Francisco, Calif., USA; Roche Professional Diagnostics, Penzberg, Germany).

Intra-assay precision was assessed by measuring samples in 12 duplicate determinations distributed within a run of at least eight hours duration performed on three different instruments. Six human sera samples with native IL-13 levels between 0.17 pg/mL and 7.5 pg/mL were used. These values were obtained using recombinant IL-13 as standards. Two additional sera were spiked with recombinant human IL-13 to examine precision of the 2- and 3-digit pg/mL concentration range. Intra-assay precision ranged from 1.5%-3.8% CV and the inter-assay precision ranged from 3.1-5.1% (Table 3).

TABLE 3
IMPACT IL-13 Assay Performance Summary
Precision

| | | Intra Assay | Inter-Assay |
|---|---|---|---|
| Control^{a} | Concentration^{c} | Precision (% CV) | Precision (% CV) |
| LLOQ1 | 0.005 | N.D.^{d} | 28.5 |
| LLOQ2 | 0.008 | N.D. | 20.4 |
| LLOQ3 | 0.011 | N.D. | 20.5 |
| LLOQ4 | 0.014 | N.D. | 14.9 |
| LLOQ5 | 0.020 | N.D. | 7.7 |
| Low | 0.178 | 2.8 | 5.1 |
| Mid^{b} | 27.5 | 3.8 | 4.3 |
| High^{b} | 355.8 | 1.5 | 3.1 |

Accuracy and Parallelism/Linearity

| | | |
|---|---|---|
| Accuracy | 30 pg/mL | 34/35 samples recover within |
| | | 80-120% of expected |
| Parallelism | Serial 1:1 (V/V) | 5/5 samples recover within |
| | dilutions | 80-120% of expected |

^{a}Serum samples containing native IL-13 were used except where noted
^{b}Serum samples containing native IL-13 were spiked with recombinant IL-13
^{c}Mean observed concentration of native IL-13 levels (n = 24 runs)
^{d}N.D.; not determined

The lower limit of quantitation (LLOQ) was determined by inter-assay precision (samples measured in duplicate, n=8 runs, three different instruments used) characterization using a dilution series with endogenous IL-13 from different donors and spiked in concentrations ranging from 0.002-0.11 pg/mL into fluids that had undetectable levels of IL-13 (horse serum, sample diluent). LLOQ is defined as the lowest concentration of IL-13 at which coefficient of variation (CV) is less than or equal to 20%. To determine the LLOQ of the assay, the precision of measurements of both native IL-13 and recombinant IL-13 samples were assessed. The LLOQ was determined by inter-assay precision and found to be 0.014 pg/mL, the lowest concentration of IL-13 at which CV<20% was achieved (Table 3).

Accuracy was assessed by spiking 30 pg/mL of recombinant human IL-13 into asthmatic patient serum samples. Samples were incubated for 1 hour at room temperature and then analyzed in the IMPACT IL-13 assay as described above. Parallelism was assessed by performing serial 1:1 (V/V) dilutions of asthmatic serum samples with assay diluent. Diluted samples were then analyzed in the IMPACT IL-13 assay as described above. Recovery ranged from 87-102% in 34 of the 35 asthmatic samples tested (Table 3). Parallelism was assessed by analyzing 5 samples in a dilution series using sample diluent. Recovery ranged between 95-105% demonstrating acceptable parallelism (Table 3).

There are conflicting reports in the scientific literature as to the presence or absence of a form of the IL-13Rα2 receptor in the peripheral blood which, if present, could potentially interfere with the IL-13 assay. Kasaian et al., J. Immunol. 187:561-9 (2011); O'Toole et al., Clin Exp Allergy 38:594-601 (2008). The potential for sIL-13Rα2 to interfere with the detection of IL-13 was assessed by the addition of 2-1000 pg/mL of sIL-13Rα2 (R&D Systems, MN, catalog no. 614-INS) to healthy control serum samples. Samples were incubated for 1 hour at room temperature and then tested in the IMPACT IL-13 assay as described above. No significant interference in the ability to detect IL-13 was detected in the presence of 2-1000 pg/mL sIL-13Rα2 (data not shown).

Impact IL-13 Assay Specificity and IL-13 Levels in Serum from Asthma, IPF, and Atopic Dermatitis Patients.

IL-13 levels in the serum samples from patients with asthma or IPF and from healthy volunteers (asthma n=34, IPF n=32, and HV n=10) were determined using the IMPACT IL-13 assay. In addition to the asthma and IPF patient samples (which were also examined using the Erenna® IL-13 Immunoassay), serum from patients with atopic dermatitis (n=25) were also examined in the IMPACT IL-13 assay. All of the samples tested had detectable levels of IL-13 levels regardless of whether they were obtained from patients with Th2-associated disease or from healthy volunteers with levels ranging from 0.11 pg/mL to 4.22 pg/mL (FIG. 4). The lowest IL-13 level detected in this set of samples was 0.11 pg/mL, more than 9 fold higher than the LLOQ.

Given the specificity issues observed with the Erenna® IL-13 Immunoassay as described above, we evaluated the specificity of the IMPACT IL-13 assay. Specificity was assessed by pre-incubating serum samples with excess levels (100 μg/mL) of the capture antibody (the first antibody) for one hour at room temperature prior to running the IMPACT IL-13 assay under standard conditions as described above. As shown in FIG. 4, the IL-13 assay signal from all samples tested (n=101) were effectively depleted to below the LLOQ level, confirming the specificity of the IL-13 measurements.

The range of serum IL-13 levels detected and quantified, and the median values, were as follows: in healthy volunteers, the range was 0.11 pg/mL to 2.25 pg/mL and the median was 0.36 pg/mL (n=50); in asthma patients, the range was 0.16 pg/mL to 2.73 pg/mL and the median was 0.64 pg/mL (n=34); in IPF patients, the range was 0.24 pg/mL to 2.15 pg/mL and the median was 0.71 pg/mL (n=32); in atopic dermatitis patients, the range was 0.17 pg/mL to 4.22 pg/mL and the median was 0.82 pg/mL (n=25) (see FIG. 5). Although the serum levels of IL-13 in healthy volunteers overlapped with the levels in the Th2-associated diseases, the median levels of serum IL-13 in each of asthma, IPF and atopic dermatitis serum samples was above that of healthy volunteers. Mann-Whitney test was performed to compare the means between HV and asthma, IPF or atopic dermatitis, respectively. The P value for each comparison was <0.0001. (FIG. 5, Table 4).

TABLE 4
Serum IL-13 levels determined by IMPACT IL-13 assay.

| | HV | Asthma | IPF | AD |
|---|---|---|---|---|
| N | 50 | 34 | 32 | 25 |
| Minimum | 0.11 | 0.16 | 0.24 | 0.17 |
| 25% Percentile | 0.27 | 0.44 | 0.45 | 0.57 |
| Median | 0.36 | 0.64 | 0.71 | 0.82 |
| 75% Percentile | 0.55 | 1.10 | 1.27 | 0.99 |
| Maximum | 2.25 | 2.73 | 2.15 | 4.22 |
| Mean | 0.48 | 0.78 | 0.86 | 1.00 |
| Lower 95% CI of mean | 0.36 | 0.60 | 0.67 | 0.61 |
| Upper 95% CI of mean | 0.59 | 0.96 | 1.04 | 1.39 |

### Comparison Between Erenna® IL-13 Immunoassay and IMPACT IL-13 Assay

Given that the Erenna® IL-13 Immunoassay uses different anti-IL-13 antibodies from those used with the IMPACT IL-13 assay, we were unable to directly compare the analytical characteristics of the Erenna® and IMPACT platforms. For instance, the significant matrix interference observed with the Erenna® IL-13 Immunoassay (Fraser S, et al., Bioanalysis 6:1123-9 [2014]) may be a reflection of the antibody reagents used for capture and detection. It is possible that similar specificity issues may have been observed if the IMPACT IL-13 assay had used utilized the same analytical reagents as the Erenna® IL-13 Immunoassay.

Nonetheless, an important aspect of a biomarker immunoassay method is the ability to detect the native biomarker in a majority of disease state samples. Therefore, a pragmatic alternative approach to direct comparison of both assays is to compare the ability of both methods to detect the native form of the biomarker in the same cohort of patient samples. Out of the 30 serum samples (HV [n=10], asthma patients [n=10] and IPF patients [n=10]) commonly tested with the modified Erenna® IL-13 Immunoassay and with the IMPACT IL-13 assay, 9 samples showed detectable IL-13 levels above the respective LLOQ of both assays. A comparison of the IL-13 levels obtained by each assay gave a Pearson's correlation coefficient of 0.65, with IMPACT IL-13 providing relatively higher levels of IL-13 compared to the modified Erenna® IL-13 Immunoassay (data not shown). The differences in IL-13 quantitation may be due to differences in reference material used in the methods. It is also possible that the detection of IL-13 using the modified Erenna® IL-13 Immunoassay was interfered by the presence of sIL-13Rα2, a possibility that we ruled out for the IMPACT IL-13 assay at the concentrations tested, as described above.

We also tested a different commercially available immunoassay that utilizes the sandwich principle (R&D Systems Quantikine® ELISA, human IL-13 immunoassay, Cat. # D1300B), which is reported to have a minimal detectable dose of IL-13 in serum between 3.46-57.4 pg/mL, at least 10-fold to 20-fold lower sensitivity than either the original Erenna® IL-13 Immunoassay or the modified Erenna® IL-13 Immunoassay described above. Following manufacturer's instructions, we found the assay standard curve precision was suboptimal below 125 pg/mL. Moreover, the IL-13 signal was not detectable in any asthma samples (0 out of 25) using the R&D Systems Quantikine® ELISA kit yet was detectable in all of the same asthma samples (25 out of 25) using the IMPACT IL-13 assay (data not shown).

In summary, we developed an immunoassay for the detection and quantitation of serum IL-13 that, surprisingly and unpredictably, was both highly sensitive and highly specific. We demonstrated that this assay, IMPACT IL-13, is able to specifically detect and accurately quantify fg/mL levels of IL-13 in serum. We believe this is the first description of an assay for serum IL-13 having such high sensitivity and specificity. The analytical reagents used in IMPACT IL-13 assay are likely key contributors to the high assay sensitivity and specificity as a number of different anti-IL-13 antibody pairs were tested and only one of those pairs met the desired assay performance metrics.

In addition, using the IMPACT IL-13 assay, we measured circulating IL-13 levels in healthy volunteers and patients with Th2-associated diseases, namely asthma, IPF, and or atopic dermatitis. We found that the majority of healthy volunteers had IL-13 levels below the median of IL-13 levels found in patients with Th2-associated disease. Accordingly, this result suggests that patient stratification for treatment with therapeutics targeting the Th2 pathway according to serum IL-13 levels may be a useful approach, a hypothesis explored in the next example.

### Example 4—Serum IL-13 Levels Predict Responsiveness to Lebrikizumab Treatment and are Prognostic for Asthma Exacerbations

It is well-documented that the different biomarkers serum periostin, blood eosinophils, FeNO and serum IgE reflect the biology of Th2 inflammation in patients with asthma. It has also been shown that each biomarker, in certain clinical studies, enriched for clinical benefit from therapeutic intervention in the Th2 pathway and additionally, in certain cases, was a prognostic biomarker or a pharmacodynamic biomarker that reflected drug efficacy (see, e.g., Anon J R, et al., AnnalsATS 2013; 10 (supplement):5206-13 [2013]; Nair et al., New Engl. J. Med. 360(10):985-93 [2009]; Pavord et al., Lancet 380(9842):651-9 [2012]; Bel et al., New Engl. J. Med. 371(13):1189-97 [2014]; Ortega et al., New Engl. J. Med. 371(13):1198-207 [2014]; Castro et al., Lancet Resp. Med. 2(11):879-90 [2014]). Using the IMPACT IL-13 assay described above, we sought to assess the relationship between peripheral IL-13 levels and other Th2 asthma biomarkers (serum periostin, blood eosinophils, FeNO and serum IgE) as well as to evaluate peripheral IL-13 levels as a lebrikizumab-predictive and disease-prognostic biomarker.

### Phase IIb Clinical Studies of Lebrikizumab

Two phase IIb studies were conducted which were replicate, randomised, multicentre, double-blind, placebo-controlled studies. Patients were randomised in a 1:1:1:1 ratio to receive lebrikizumab 37.5 mg, 125 mg, 250 mg, or placebo subcutaneously every 4 weeks. Randomisation was stratified by baseline serum periostin, history of asthma exacerbations within the last 12 months and baseline asthma medications. All patients were to remain on their standard of care therapy that consisted of 500-2000 μg/day ICS therapy (fluticasone propionate DPI or equivalent) and a second eligible asthma controller medication. The studies are described in further detail, along with results in Hanania et al., Journal of Allergy and Clinical Immunology, Volume 133, Issue 2, AB402 (2014) (abstract).

Patients aged 18-75 years with asthma, who remained uncontrolled despite daily use of 500-2000 μg/day of fluticasone propionate DPI or equivalent and a second asthma controller medication were eligible for inclusion in the studies. Eligible second controller medications included long-acting β agonist, leukotriene receptor antagonist, long-acting muscarinic antagonist, or theophylline.

Inclusion criteria included: diagnosis of asthma ≥12 months; bronchodilator response (≥12% relative improvement); prebronchodilator FEV₁ 40%-80% of predicted. Uncontrolled asthma was defined as an Asthma Control Questionnaire-5 (ACQ-5) score ≥1.5 and at least one of the following: symptoms >2 days/week; night-time awakenings ≥1 time/week; use of a short-acting β agonist as rescue medication >2 days/week; or interference with normal daily activities. Patients were excluded if they had received maintenance oral corticosteroid treatment within the previous 3 months or treatment with systemic corticosteroids within the previous 4 weeks for any reason. Exclusion criteria included: history of a severe allergic reaction or anaphylactic reaction to a biologic agent or known hypersensitivity to any component of the lebrikizumab injection; maintenance oral corticosteroid therapy, defined as daily or alternate day oral corticosteroid maintenance therapy within the 3 months prior to Visit 1; treatment with systemic (e.g. oral, IV, or IM) corticosteroids within the 4 weeks prior to Visit 1 or at any time during the screening period for any reason, including an acute exacerbation event, or treatment with intraarticular corticosteroids within the 4 weeks prior to Visit 1 or at any time during the screening period; a major episode of infection; known immunodeficiency, including, but not limited to, HIV infection; evidence of acute or chronic hepatitis or known liver cirrhosis; history of cystic fibrosis, COPD, and/or other clinically significant lung disease other than asthma; known current malignancy or current evaluation for a potential malignancy; current smoker, or former smoker with a smoking history of >10 pack-years; current use of an immunomodulatory/immunosuppressive therapy or past use within 3 months or 5 drug half-lives prior to Visit 1; use of a biologic therapy including omalizumab at any time during the 6 months prior to Visit 1; use of zileuton or roflumilast at any time during the 4 weeks prior to Visit 1; traditional herbal medicine for treatment of allergic disease or asthma within the 3 months prior to Visit 1; initiation of or change in allergen immunotherapy within the 3 months prior to Visit 1; treatment with an investigational agent within the 30 days prior to Visit 1 (or 5 half-lives of the investigational agent, whichever is longer); receipt of a live attenuated vaccine within the 4 weeks prior to Visit 1; body mass index >38 kg/m²; body weight <40 kg.

The following efficacy and safety assessments were evaluated in these studies. The primary endpoint was the rate of asthma exacerbations during the placebo-controlled period. An asthma exacerbation was defined as new or increased asthma symptoms that led to treatment with systemic corticosteroids or to hospitalisation. Treatment with systemic corticosteroids was defined as oral, intravenous (IV), or intramuscular (IM) corticosteroid treatment for ≥3 days or an emergency room visit with ≥1 dose of IV or IM corticosteroids. Asthma exacerbations were assessed at each study visit by the investigator using directed questions to assess whether the patient had experienced any asthma exacerbations since the last visit. It was pre-specified that the primary and all secondary endpoints would be evaluated separately in the periostin-high and periostin-low groups (based on a cut-point of 50 ng/mL serum periostin). Spirometry (pre- and post-bronchodilator) was assessed throughout the study. Spirometric measures collected included FEV₁, FVC (volume in litres) and PEF (litres per minute). The percentage of predicted FEV₁ and FVC was derived from these volume measurements using the equations derived from the National Health and Nutrition Examination Survey dataset as described by Hankinson et al., Am J Respir Crit Care Med 159:179-87 (1999). The acceptability of the data, including the graphic representations of the manuvers, was determined by blinded over-readers. Calculations for the reproducibility of the acceptable manuvers were programmed. The last dose of a short-acting bronchodilator had to be at least 4 hours before testing, the last dose of a LABA at least 12 hours before testing, and the last dose of a LAMA at least 24 hours before testing. For patients who were not properly prepared for testing (e.g. had taken a bronchodilator before arrival), the visit was rescheduled. Measurement of spirometry was performed on a computerised spirometry system, Vitalograph® Spirotrac® with 6800 Spirometer (Vitalograph; Ennis, Ireland) configured to the requirements of the study and in accordance with guidelines published by the ATS/ERS Standardisation of Spirometry (Miller et al., Eur Respir J 26:319-38 [2005]). A peak flow/eDiary device was used for once daily measurement of peak expiratory flow (PEF) (between 5 am and 11 am) and recording of asthma rescue medication and controller use. Patients were provided with a hand-held peak flow/diary device, Vitalograph® 2120 In2itive e-Diary (Vitalograph), for once daily PEF measurements and e-Diary recording of asthma rescue and controller medication use during the study.

Pre-specified secondary endpoints were relative change in pre-bronchodilator FEV₁ from baseline to Week 52, time to first asthma exacerbation during the placebo-controlled period, change from baseline to Week 52 in the asthma-specific health-related quality of life measure, Asthma Quality-of-Life Questionnaire (Standardised; [AQLQ(S)]), change in asthma rescue medication use from baseline to Week 52, rate of urgent asthma-related health care utilisation (i.e. hospitalisations, emergency department visits, and acute care visits) during the placebo-controlled period. Safety endpoints were the rate and severity of adverse events (AEs) during the placebo-controlled and follow-up periods and the incidence of anti-therapeutic antibodies (ATAs) during the study relative to baseline. The immunogenicity of lebrikizumab was assessed using a tiered ATA analysis strategy.

Biomarker assessments were carried out as follows. FeNO was assessed at baseline and at each subsequent study visit, using a hand-held portable device (NIOX Aerocrine; Solna, Sweden) in accordance with the American Thoracic Society guidelines (ATS/ERS Recommendations, Am J Respir Crit Care Med 171:912-30 [2005]). Serum to evaluate periostin levels was collected at screening, baseline, and each subsequent study visit. Periostin was measured using the Roche Elecsys® Periostin assay (Roche Diagnostics, Penzberg Germany) on the Cobas e601 platform, which is an electrochemiluminescence immunoassay, using the sandwich principle. Patients, physicians and site staff were blinded to FeNO and periostin values during the study. Hematological assessments, including peripheral blood eosinophil counts and serum IgE levels, were performed according to standard clinical laboratory procedures at screening, baseline, and at each subsequent study visit beginning at Week 4 using a central lab and sites were blinded from randomisation.

As reported by Hanania et al., Journal of Allergy and Clinical Immunology, Volume 133, Issue 2, AB402 (2014) (abstract), in patients with uncontrolled severe asthma despite inhaled corticosteroid therapy and an additional controller, lebrikizumab administered subcutaneously every 4 weeks reduced asthma exacerbation rate by 60% (95% CI 18, 80) compared with placebo in periostin-high patients and by 5% (95% CI-81, 47) in periostin-low patients. In addition, lebrikizumab positively impacted lung function, as measured by change in FEV₁, in periostin-high patients. Lebrikizumab was generally well tolerated and no clinically important safety signals were observed. These results extend the findings described previously and support the finding that baseline periostin level can be predictive of lebrikizumab treatment benefit.

With respect to biomarkers FeNO, peripheral blood eosinophils, and serum periostin, the following results were reported. Baseline FeNO levels were lower in the placebo and lebrikizumab 37.5 mg groups, as well as in periostin-low patients. The changes relative to placebo at Week 12 in periostin-high patients were −3.9 to −12.5 parts per billion (ppb) across the different lebrikizumab dose groups. At Week 12 in periostin-low patients the differences between the means in FeNO were −8.9 to −11.0 ppb across the lebrikizumab dose groups relative to placebo. Baseline levels of peripheral blood eosinophils were well balanced across different treatment arms. At Week 12 there was a small increase in absolute blood eosinophil levels with lebrikizumab, particularly in periostin-high subjects. The placebo-corrected change ranged from 0.29 to 0.56×10³/μL in periostin-high patients and from −0.01 to 0.07×10³/μL in the periostin-low group. In the periostin-high group, the increase in peripheral blood eosinophils appeared to be dose dependent, with the 37.5 mg demonstrating the smallest changes. Changes in peripheral blood eosinophil counts have been reported previously (Corren J, et al., N Engl J Med 365:1088-98 [2011]; Scheerens H, et al., [abstract] Am J Respir Crit Care Med 185:PA3960 [2012]), and may reflect blocking of IL-13 activity The increased eosinophil counts in blood may be due to decreased migration from blood to the airways, due to reduced chemotaxis (Blanchard C, et al., Mucosal Immunol 1:289-96 [2008]; Johansson M W, et al., Am J Respir Cell Mol Biol 48:503-10 [2013]). Baseline levels of serum periostin were also well balanced across different treatment arms with a median (Day −7) value across all groups of 47.9 ng/mL. At Week 12, following lebrikizumab treatment, there was a placebo-corrected decrease of 3.7-8.3% in periostin in periostin-high subjects and little change in periostin-low subjects. There was no clear evidence of dose-dependent changes in periostin levels.

### Serum IL-13 Measurements and Analyses

The IMPACT IL-13 assay was used to determine serum IL-13 levels at baseline (week 0) in a total of 329 patient serum samples from the phase IIb studies described above. Each serum sample was measured in duplicate following the IMPACT IL-13 assay methods described above. The final IL-13 level in each sample was reported as the mean concentration of duplicate measurements with percentage coefficient of variation (% CV)≤15%. The duplicate measurements with % CV >15% were considered as invalid and excluded from the analysis. In addition, the spike recovery for each sample was evaluated. 30 pg/mL of recombinant human IL-13 was spiked into each endogenous sample aliquot before the assay procedures. The samples with <80% spike recovery were considered measurement invalid and excluded from the analysis.

The following statistical analyses were carried out. Nonparametric Spearman rank-order correlation analyses for serum IL-13, blood eosinophils, serum periostin, FeNO, and serum IgE levels at the baseline were performed using JMP 10.0.2 (SAS Institute, Cary, N.C.). IL-13 high and IL-13 low subgroups were stratified by the median level of serum IL-13 in the phase IIb patient samples measured. The FEV₁ mean (SE) percentage changes from baseline were calculated according to study groups at the week 1, 4, 8 and 12. The week 12 analysis was used for FEV₁ efficacy evaluation. The mean percentage changes from baseline were compared between each treatment group, respectively and placebo group by t test assuming unequal variances. The differences between the means and the associated two-sided 95% confidence intervals were calculated accordingly. The rates of protocol-defined exacerbations of asthma during placebo-controlled treatment period were estimated by dividing the total number of such exacerbations over the treatment period by the total time (years) of the treatment period in each group. The exacerbation rates between each treatment group, respectively and placebo group were compared using Poisson regression model with overdispersion. The two-sided 95% confidence intervals for exacerbation rate reduction between treatment group and placebo group were reported. For the prognostic analysis, IL-13 was added as a continuous covariate to the Poisson regression model fit to the data from placebo patients. For this analysis, the extreme observation of 42.93 pg/mL IL13 was removed to avoid undue influence. A sensitivity analysis removing another influential value of 8.5 pg/mL yielded consistent results.

Serum IL-13 Results and Correlation with Other Th2 Biomarkers

As stated above, we measured serum IL-13 levels in 329 patient samples. Four samples had invalid measurements and were excluded from the further analysis, specifically, one that had undetectable levels, one that had % CV more than 15% between duplicate measurements, and two that had spike recovery less than 80%. Therefore, the IL-13 detection rate was considered as 98.5% (324 out of 329). The serum IL-13 levels in these 324 patient samples ranged from 0.053 to 42.935 pg/mL with a median of 0.785 pg/mL. The mean (95% CI) level was 1.172 pg/mL (0.898, 1.446). (Table 5).

TABLE 5
Biomarker levels distribution in phase IIb studies at baseline.

| | IL-13 | Eosinophils | Periostin | FeNO | IgE |
|---|---|---|---|---|---|
| | (pg/mL) | (×10\hat{ }9/L) | (ng/mL) | (ppb) | (IU/mL) |
| N | 324 | 324 | 324 | 315 | 324 |
| Minimum | 0.053 | 0.01 | 25.17 | 2.50 | 4.00 |
| 25% percentile | 0.522 | 0.15 | 40.10 | 13.00 | 54.25 |
| Median | 0.785 | 0.25 | 47.61 | 20.00 | 159.50 |
| 75% percentile | 1.297 | 0.36 | 55.69 | 32.00 | 357.75 |
| Maximum | 42.935 | 1.64 | 112.30 | 229.00 | 5000.00 |
| Mean (95% CI) | 1.172 (0.898, 1.446) | 0.29 (0.27, 0.32) | 49.64 (48.11, 51.17) | 28.41 (25.32, 31.50) | 376.49 (297.83, 455.16) |

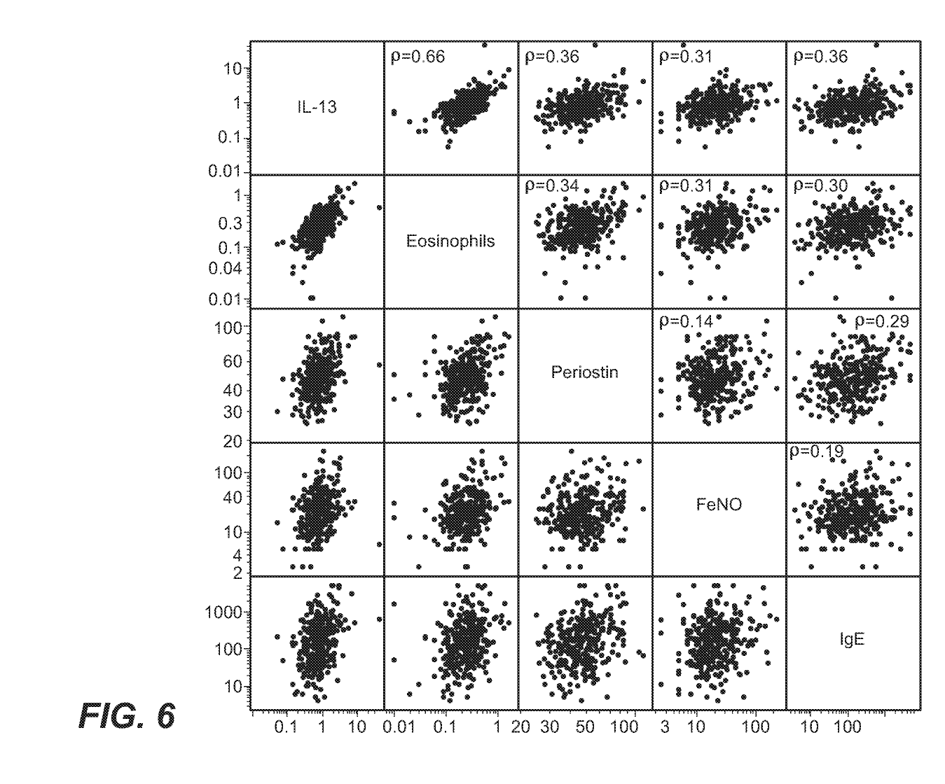

We next assessed the correlation of serum IL-13 levels with other Th2 biomarkers, specifically, blood eosinophils, serum periostin, FeNO, serum IgE. As shown in FIG. 6, at the baseline (Week 0), serum IL-13 levels strongly correlated with blood eosinophils counts, but weakly correlated with serum periostin, FeNO and serum IgE levels. The Spearman rank-order correlation coefficient (p) between serum IL-13 and blood eosinophils was 0.66, while the Spearman's p between IL-13 and serum periostin, FeNO and serum IgE were 0.36, 0.31 and 0.36, respectively. While weak correlations between eosinophils, periostin, FeNO and serum IgE have been described previously and were therefore expected, the strong correlation between serum IL-13 and blood eosinophil levels was surprising and unexpected and to our knowledge, has not been previously described. This strong correlation indicates that the combination of serum IL-13 and blood eosinophil levels are particularly informative biomarkers for studying Th2-associated diseases and therapeutics targeting the Th2 pathway, in addition to each biomarker alone.

### Baseline Serum IL-13 Levels Predicts Responsiveness to Lebrikizumab Treatment

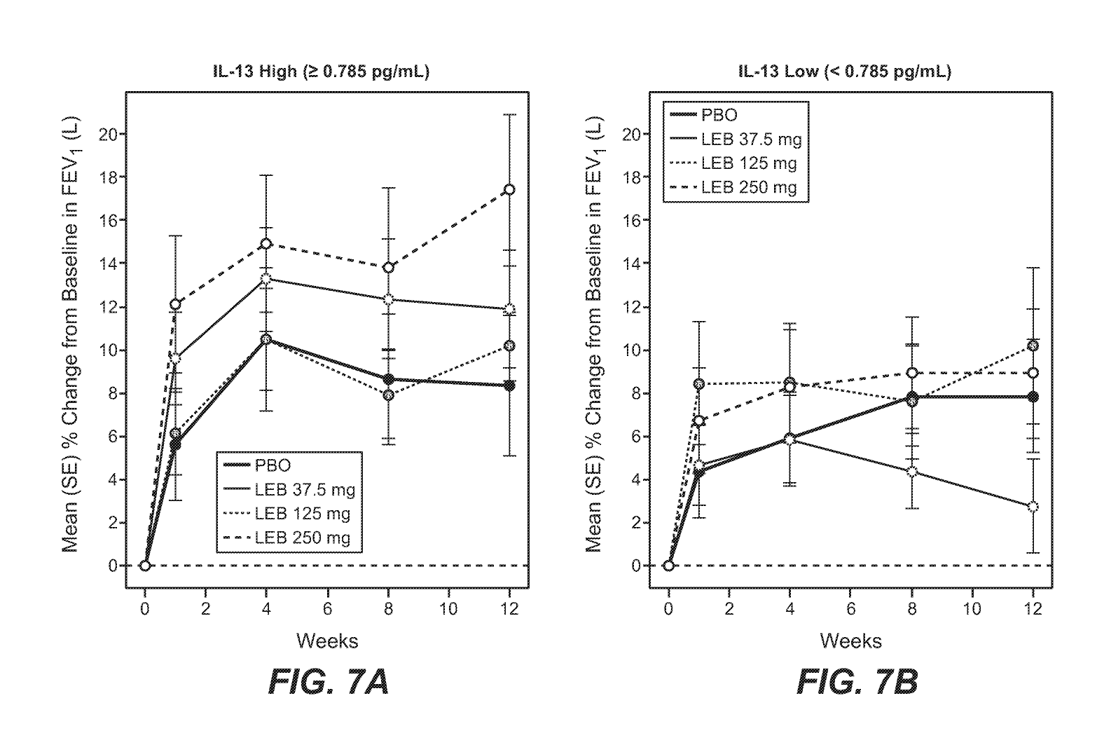

Using the serum IL-13 median of 0.785 pg/mL, patients were stratified as serum IL-13 high (serum IL-13 ≥0.785 pg/mL) or serum IL-13 low (serum IL-13<0.785 pg/mL). FIG. 7A (serum IL-13 high) and FIG. 7B (serum IL-13 low) show the mean percentage change in FEV₁ at Week 12 compared to baseline FEV₁. At Week 12, the placebo-normalized mean increases from baseline FEV₁ in the 37.5 mg and 250 mg lebrikizumab arms were greater in the serum IL-13 high group than serum IL-13 low group. For the 37.5 mg lebrikizumab arm, the improvement was 3.51% (−4.98, 12.00) in serum IL-13 high versus −5.11% (−11.99, 1.77) in serum IL-13 low group. For the 250 mg lebrikizumab arm, the improvement was 9.04% (−0.54, 18.62) in the serum IL-13 high group versus 1.05% (−7.00, 9.11) in the serum IL-13 low group. The improvement of FEV₁ in the 125 mg lebribikizumab arm was similar between the serum IL-13 high and serum IL-13 low groups. (FIG. 7A and FIG. 7B, Table 6).

TABLE 6
Mean percentage change from baseline in FEV₁ at
Week 12 versus placebo by serum IL-13 status.

| | IL-13 high (≥0.785 pg/mL) | IL-13 low (<0.785 pg/mL) |
|---|---|---|
| LEB | 3.51% (95% CI: −4.98, 12.00) | −5.11% (95% CI: −11.99, 1.77) |
| 37.5 mg | | |
| LEB | 1.82% (95% CI: −5.49, 9.12) | 2.33% (95% CI: −6.68, 11.34) |
| 125 mg | | |
| LEB | 9.04% (95% CI: −0.54, 18.62) | 1.05% (95% CI: −7.00, 9.11) |
| 250 mg | | |

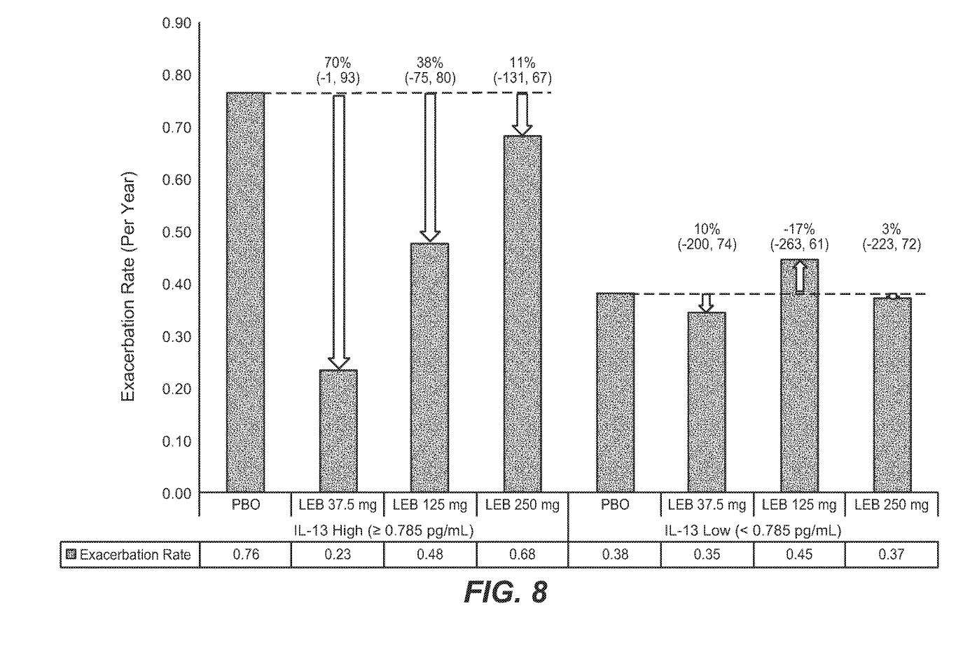

FIG. 8 shows that the estimated exacerbation rate reduction during the placebo-controlled treatment period was greater in the serum IL-13 high group than in the serum IL-13 low group. In the serum IL-13 high group, the exacerbation rate reduction was 70% (95% CI: −1% to 93%), 38% (95% CI: −75% to 80%) and 11% (95% CI: −131% to 67%) in 37.5 mg, 125 mg and 250 mg lebrikizumab arms, respectively. While in the serum IL-13 low group, the exacerbation rate reduction was 10% (95% CI: −200% to 74%), −17% (95% CI: −263% to 61%) and 3% (95% CI: −223% to 72%) in 37.5 mg, 125 mg and 250 mg lebrikizumab arms, respectively.

We also examined whether serum IL-13 level was prognostic for asthma exacerbations. The exacerbation rate in the placebo arms were 0.76 in the serum IL-13 high group and 0.38 in the serum IL-13 low group (FIG. 8). This result suggests that the baseline level of serum IL-13 is a prognostic biomarker of exacerbations in patients with uncontrolled asthma despite standard of care. To confirm this conclusion, a Poisson regression allowing for overdispersion was fit to the data from placebo patients. This analysis yielded a factor of 1.03 (95% CI: 1.01 to 1.05) for an increase in the yearly exacerbation rate for a 0.1 pg/mL increase in IL-13, further supporting the conclusion that serum IL-13 level is prognostic for exacerbations.

In summary, baseline levels of serum IL-13 in samples from 329 asthma patients in phase IIb clinical studies of lebrikizumab were measured using the IMPACT IL-13 assay. The IL-13 detection rate in these serum samples was 98.5% (324 out of 329). The median level was 0.785 pg/mL. As described above, the baseline levels of serum IL-13 strongly correlated with blood eosinophils counts, and weakly correlated with serum periostin, FeNO and serum IgE levels. In addition, serum IL-13 level was predictive of patient responsiveness to lebrikizumab treatment: patients in the high serum IL-13 group demonstrated greater clinical benefit from lebrikizumab treatment as assessed by exacerbation rate reduction and FEV₁ improvement than those in the low serum IL-13 group. Finally, we also demonstrated that serum IL-13 level is a prognostic biomarker for asthma exacerbations with patients in the placebo arm, high serum IL-13 group showing a higher rate of exacerbations compared to patients in the placebo arm, low serum IL-13 group.

Given certain limitations and inconveniences associated with previously-described Th2 biomarker assessments, the development of a serum IL-13 assay that has both high sensitivity and high specificity, as described herein, and the demonstration herein that serum IL-13 levels are predictive of therapeutic benefit with a therapeutic agent targeting the Th2 pathway and also prognostic for asthma exacerbations, represent an important advance in the field.

TABLE OF SEQUENCES

| SEQ | | |
|---|---|---|
| ID NO: | Description | Sequence |
| 1 | lebrikizumab | VTLRESGPAL VKPTQTLTLT CTVSGFSLSA YSVNWIRQPP |
| | heavy chain | GKALEWLAMI WGDGKIVYNS ALKSRLTISK DTSKNQVVLT |
| | variable region | MTNMDPVDTA TYYCAGDGYY PYAMDNWGQG SLVTVSS |
| 2 | lebrikizumab | DIVMTQSPDS LSVSLGERAT INCRASKSVD SYGNSFMHWY |
| | light chain | QQKPGQPPKL LIYLASNLES GVPDRFSGSG SGTDFTLTIS |
| | variable region | SLQAEDVAVY YCQQNNEDPR TFGGGTKVEI K |
| 3 | Alternate | QVTLRESGPA LVKPTQTLTL TCTVSGFSLS AYSVNWIRQP |
| | lebrikizumab VH | PGKALEWLAM IWGDGKIVYN SALKSRLTIS KDTSKNQVVL |
| | | TMTNMDPVDT ATYYCAGDGY YPYAMDNWGQ GSLVTVSS |
| 4 | Alternate | DIVMTQSPDS LSVSLGERAT INCRASKSVD SYGNSFMHWY |
| | lebrikizumab VL | QQKPGQPPKL LIYLASNLES GVPDRFSGSG SGTDFTLTIS |
| | | SLQAEDVAVY YCQQNNEDPR TFGGGTKVEI KR |
| 5 | lebrikizumab | AYSVN |
| | HVR-H1 | |
| 6 | lebrikizumab | MIWGDGKIVYNSALKS |
| | HVR-H2 | |
| 7 | lebrikizumab | DGYYPYAMDN |
| | HVR-H3 | |
| 8 | lebrikizumab | RASKSVDSYGNSFMH |
| | HVR-L1 | |
| 9 | lebrikizumab | LASNLES |
| | HVR-L2 | |
| 10 | lebrikizumab | QQNNEDPRT |
| | HVR-L3 | |
| 11 | anti-IL-13 | DIVLTQSPAS LAVSLGQRAT ISCRASQSVS TSSYSYMNWY |
| | mu11H4 VL | QQTPGQPPKL LIKYASNLES GIPARFSGSG SGTDFTLNIH |
| | | PVEEEDTATY YCQHSWEIPY TFGGGT |
| 12 | anti-IL-13 | QVTLKESGPG ILQPSQTLSL TCSFSGFSLS TSDMGVGWIR |
| | mu11H4 VH | QPSGKGLEWL AHIWWDDVKR YNPALKSRLT ISKDTSSSQV |
| | | FLKIASVDTA DTATYYCARI GTNYGYDGLF DYWGQGTTLT VSS |
| 13 | anti-IL-13 | GFSLSTSDMGVG |
| | M1111H4 HVRH1 | |
| 14 | anti-IL-13 | AHIWWDDVKRYNPALKS |
| | mu11H4 HVRH2 | |
| 15 | anti-IL-13 | ARIGTNYGYDGLFDY |
| | mu11H4 HVRH3 | |
| 16 | anti-IL-13 | RASQSVSTSSYSYMN |
| | mu11H4 HVRL1 | |
| 17 | anti-IL-13 | YASNLES |
| | mu11H4 HVRL2 | |
| 18 | anti-IL-13 | QHSWEIPYT |
| | mu11H4 HVRL3 | |
| 19 | human IL-13, | SPGPVPPSTA LRELIEELVN ITQNQKAPLC NGSMVWSINLTA |
| | mature form | GMYCAALESL INVSGCSAIE KTQRMLSGFC PHKVSAGQFS |
| | (without signal | SLHVRDTKIE VAQFVKDLLL HLKKLFREGR FN |
| | sequence) | |
| 20 | IL-13 epitope, | ESLINVSG |
| | amino acids 50 to | |
| | 57 of SEQ ID | |
| | NO: 19 | |
| 21 | IL-13 epitope, | YCAALESLINVS |
| | amino acids 45 to | |
| | 56 of SEQ ID | |
| | NO: 19 | |
| 22 | anti-IgE antibody | Asp Ile Gln Leu Thr Gln Ser Pro Ser Ser Leu Ser |
| | light chain | Ala Ser Val Gly Asp Arg Val Thr Ile Thr Cys Arg |
| | variable region | Ala Ser Gln Ser Val Asp Tyr Asp Gly Asp Ser Tyr |
| | | Met Asn Trp Tyr Gln Gln Lys Pro Gly Lys Ala Pro |
| | | Lys Leu Leu Ile Tyr Ala Ala Ser Tyr Leu Glu Ser |
| | | Gly Val Pro Ser Arg Phe Ser Gly Ser Gly Ser Gly |
| | | Thr Asp Phe Thr Leu Thr Ile Ser Ser Leu Gln Pro |
| | | Glu Asp Phe Ala Thr Tyr Tyr Cys Gln Gln Ser His |
| | | Glu Asp Pro Tyr Thr Phe Gly Gln Gly Thr Lys Val |
| | | Glu Ile Lys Arg Thr Val |
| 23 | anti-IgE antibody | Glu Val Gln Leu Val Glu Ser Gly Gly Gly Leu Val |
| | heavy chain | Gln Pro Gly Gly Ser Leu Arg Leu Ser Cys Ala Val |
| | variable region | Ser Gly Tyr Ser Ile Thr Ser Gly Tyr Ser Trp Asn |
| | | Trp Ile Arg Gln Ala Pro Gly Lys Gly Leu Glu Trp |
| | | Val Ala Ser Ile Thr Tyr Asp Gly Ser Thr Asn Tyr |
| | | Asn Pro Ser Val Lys Gly Arg Ile Thr Ile Ser Arg |
| | | Asp Asp Ser Lys Asn Thr Phe Tyr Leu Gln Net Asn |
| | | Ser Leu Arg Ala Glu Asp Thr Ala Val Tyr Tyr Cys |
| | | Ala Arg Gly Ser His Tyr Phe Gly His Trp His Phe |
| | | Ala Val Trp Gly Gln Gly |
| 24 | lebrikizumab VH | EVTLRESGPA LVKPTQTLTL TCTVSGFSLS AYSVNWIRQP |
| | Q1E | PGKALEWLAM IWGDGKIVYN SALKSRLTIS KDTSKNQVVL |
| | | TMTNMDPVDT ATYYCAGDGY YPYAMDNWGQ GSLVTVSS |
| 25 | lebrikizumab VL | DIVLTQSPDS LSVSLGERAT INCRASKSVD SYGNSFMHWY |
| | M4L | QQKPGQPPKL LIYLASNLES GVPDRFSGSG SGTDFTLTIS |
| | | SLQAEDVAVY YCQQNNEDPR TFGGGTKVEI KR |
| 26 | anti-IL-17 | DYAMH |
| | HVR-H1 | |
| 27 | anti-IL-17 | GINWSSGGIGYADSVKG |
| | HVR-H2 | |
| 28 | anti-IL-17 | DIGGFGEFYWNFGL |
| | HVR-H3 | |
| 29 | anti-IL-17 | RASQSVRSYLA |
| | HVR-L1 | |
| 30 | anti-IL-17 | DASNRAT |
| | HVR-L2 | |
| 31 | anti-IL-17 | QQRSNWPPAT |
| | HVR-L3 | |
| 32 | anti-IL-17 heavy | EVQLVESGGG LVQPGRSLRL SCAASGFTFD DYAMHWVRQA |
| | chain variable | PGKGLEWVSG INWSSGGIGY ADSVKGRFTI SRDNAKNSLY |
| | region | LQMNSLRAED TALYYCARDI GGFGEFYWNF GLWGRGTLVT VSS |
| 33 | anti-IL-17 light | EIVLTQSPAT LSLSPGERAT LSCRASQSVR SYLAWYQQKP |
| | chain variable | GQAPRLLIYD ASNRATGIPA RFSGSGSGTD FTLTISSLEP |
| | region | EDFAVYYCQQ RSNWPPATFG GGTKVEIK |

1. An immunoassay method for detecting and quantifying IL-13, wherein the method is capable of detecting and quantifying IL-13 in a sample with high sensitivity and high specificity.

2. The method of claim 1, wherein the sensitivity is determined as a lower limit of quantification (LLOQ), wherein the LLOQ is between 0.1 fg/mL and 35 fg/mL, or between 1 fg/mL and 30 fg/mL, or between 5 fg/mL and 25 fg/mL or between 10 fg/mL and 20 fg/mL.

3. The method of claim 2, wherein the LLOQ is 14 fg/mL.

4. The method of claim 1, wherein the method is a sandwich immunoassay method and comprises a first monoclonal capture antibody that specifically binds IL-13 and a second monoclonal detection antibody that specifically binds IL-13, wherein the first antibody binds a different epitope than the second antibody.

5. The method of claim 4, wherein the specificity is determined by an antigen depletion method, wherein the depletion method comprises incubation of the sample with an excess amount of the first antibody prior to performing the immunoassay method.

6. The method of claim 5, wherein antigen in the sample is completely depleted thereby producing a signal below the LLOQ in the immunoassay method.

7. The method of claim 5, wherein the sample comprises soluble IL-13Rα2 and the soluble IL-13Rα2 does not interfere with the sensitivity or specificity of the immunoassay method.

8. The method of claim 4, wherein the first antibody comprises a variable region comprising a variable heavy chain region comprising HVR-H1 comprising the amino acid sequence of SEQ ID NO: 5, HVR-H2 comprising the amino acid sequence of SEQ ID NO: 6, and HVR-H3 comprising the amino acid sequence of SEQ ID NO: 7 and a variable light chain region comprising HVR-L1 comprising the amino acid sequence of SEQ ID NO: 8, HVR-L2 comprising the amino acid sequence of SEQ ID NO: 9, and HVR-L3 comprising the amino acid sequence of SEQ ID NO: 10.

9. The method of claim 8, wherein the first antibody comprises a variable region comprising a variable heavy chain region comprising the amino acid sequence of SEQ ID NO: 1 and a variable light chain region comprising the amino acid sequence of SEQ ID NO: 2.

10. The method of claim 4, wherein the second antibody comprises a variable region comprising a variable heavy chain region comprising HVR-H1 comprising the amino acid sequence of SEQ ID NO: 13, HVR-H2 comprising the amino acid sequence of SEQ ID NO: 14, and HVR-H3 comprising the amino acid sequence of SEQ ID NO: 15 and a variable light chain region comprising HVR-L1 comprising the amino acid sequence of SEQ ID NO: 16, HVR-L2 comprising the amino acid sequence of SEQ ID NO: 17, and HVR-L3 comprising the amino acid sequence of SEQ ID NO: 18.

11. The method of claim 10, wherein the second antibody comprises a variable region comprising a variable heavy chain region comprising the amino acid sequence of SEQ ID NO: 12 and a variable light chain region comprising the amino acid sequence of SEQ ID NO: 11.

12. The method of claim 4, wherein the first antibody is an antibody fragment.

13. The method of claim 12, wherein the antibody fragment is selected from Fab, F(ab′)₂, Fab′, and Fv.

14. The method of claim 4, further comprising a third antibody, wherein the third antibody specifically binds to the second antibody and is detectably labeled.

15. The method of claim 14, wherein the second antibody is labeled with a hapten and the third antibody is an anti-hapten antibody.

16. The method of claim 15, wherein the hapten is digoxigenen and the anti-hapten antibody is an anti-digoxigenin monoclonal antibody conjugated with fluorescent latex.

17. The method of claim 1, wherein the sample is serum.

18. The method of claim 17, wherein the sample is human serum.

19. A method of predicting the response of a patient suffering from asthma or a Th2-associated disease to a therapy comprising a Th2 pathway inhibitor, the method comprising:
obtaining a biological sample from the patient,
measuring the level of IL-13 using the method of claim 1,
comparing the IL-13 level detected in the sample to a reference level,
and
predicting that the patient will respond to the therapy when the IL-13 level measured in the sample is elevated compared to the reference level or predicting that the patient will not respond to the therapy when the IL-13 level measured in the sample is reduced compared to the reference level.

20. A method of predicting responsiveness of a patient suffering from asthma or a Th2-associated disease to a therapy comprising a Th2 pathway inhibitor, the method comprising measuring the IL-13 level in a biological sample from the patient using the method of claim 1, wherein elevated IL-13 level compared to a reference level identifies the patient as one who is likely to respond to the Th2 pathway inhibitor treatment.

21. A method of identifying a patient suffering from asthma or a Th2-associated disease as likely to respond to a therapy comprising a Th2 pathway inhibitor, the method comprising:
(a) measuring the IL-13 level in a biological sample from the patient using the method of claim 1;
(b) comparing the IL-13 level measured in (a) to a reference level; and
(c) identifying the patient as more likely to respond to the therapy comprising the Th2 pathway inhibitor when the IL-13 level measured in (a) is above the reference level.

22. The method according to claim 19, wherein the Th2 pathway inhibitor is an inhibitor of ITK, BTK, IL-9 (e.g., MEDI-528), IL-5 (e.g., Mepolizumab, CAS No. 196078-29-2; resilizumab), IL-13 (e.g., IMA-026, IMA-638 (also referred to as, anrukinzumab, INN No. 910649-32-0; QAX-576; IL4/IL13 trap), tralokinumab (also referred to as CAT-354, CAS No. 1044515-88-9); AER-001, ABT-308 (also referred to as humanized 13C5.5 antibody), IL-4 (e.g., AER-001, IL4/IL13 trap), IL-17, OX40L, TSLP, IL-25, IL-33 and IgE (e.g., XOLAIR®, QGE-031; MEDI-4212; quilizumab); and receptors such as: IL-9 receptor, IL-5 receptor (e.g., MEDI-563 (benralizumab, CAS No. 1044511-01-4), IL-4receptor alpha (e.g., AMG-317, AIR-645, dupilumab), IL-13receptoralpha1 (e.g., R-1671) and IL-13receptoralpha2, OX40, TSLP-R, IL-7Ralpha (a co-receptor for TSLP), IL17RB (receptor for IL-25), ST2 (receptor for IL-33), CCR3, CCR4, CRTH2 (e.g., AMG-853, AP768, AP-761, MLN6095, ACT129968), FcepsilonRI, FcepsilonRII/CD23 (receptors for IgE), Flap (e.g., GSK2190915), Syk kinase (R-343, PF3526299); CCR4 (AMG-761), TLR9 (QAX-935), or is a multi-cytokine inhibitor of CCR3, IL5, IL3, GM-CSF (e.g., TPI ASM8).

23. The method according to claim 22, wherein the Th2 pathway inhibitor is an IL-13 pathway inhibitor or an anti IgE binding agent.

24. The method according to claim 23, wherein the Th2 pathway inhibitor is an anti-IL-13 antibody or an anti-IL-13 bispecific antibody.

25. The method according to claim 24, wherein the anti-IL-13 antibody is an antibody comprising a VH comprising a sequence selected from SEQ ID NOs: 1, 3, and 24, and a VL comprising a sequence selected from SEQ ID NO: 2, 4, and 25; an anti-IL13 antibody comprising HVRH1, HVRH2, HVRH3, HVRL1, HVRL2, and HVRL3, wherein the respective HVRs have the amino acid sequence of SEQ ID NO.: 5, SEQ ID NO.: 6, SEQ ID NO.: 7, SEQ ID NO.: 8, SEQ ID NO.: 9, and SEQ ID NO.: 10; or lebrikizumab.

26. The method according to claim 24, wherein the anti-IL-13 bispecific antibody comprises an anti-IL-13 VH/VL unit comprising a VH comprising a sequence selected from SEQ ID NOs: 1, 3, and 24, and a VL comprising a sequence selected from SEQ ID NO: 2, 4, and 25; or an anti-IL13 VH/VL unit comprising HVRH1, HVRH2, HVRH3, HVRL1, HVRL2, and HVRL3, wherein the respective HVRs have the amino acid sequence of SEQ ID NO.: 5, SEQ ID NO.: 6, SEQ ID NO.: 7, SEQ ID NO.: 8, SEQ ID NO.: 9, and SEQ ID NO.: 10.

27. The method of claim 24, wherein the anti-IL-13 bispecific antibody is an anti-IL-4/anti-IL-13 bispecific antibody or an anti-IL-13/anti-IL-17 bispecific antibody.

28-29. (canceled)

30. The method according to claim 23, wherein the Th2 pathway inhibitor is an anti-IgE antibody.

31. The method according to claim 30, wherein the anti-IgE antibody is (i) the XOLAIR® antibody or (ii) an anti-IgE antibody comprising a variable heavy chain region and a variable light chain region, wherein the variable heavy chain region is SEQ ID NO:22 and the variable light chain region is SEQ ID NO:23.

32. A method of treating a patient having asthma or a Th2-associated disease, the method comprising:
(a) measuring the level of IL-13 in a biological sample from the patient using the method of claim 1;
(b) comparing the IL-13 level measured in (a) to a reference level;
(c) identifying the patient as more likely to respond a therapy comprising a Th2 pathway inhibitor when the IL-13 level measured in (a) is above the reference level; and
(d) administering the therapy when the IL-13 level measured in (a) is above the reference level, thereby treating the asthma or Th2-associated disorder.

33-34. (canceled)

35. The method of any one of claims 19-21, wherein the reference level is the median level of IL-13 in a reference population.

36. The method of claim 32, wherein the Th2 pathway inhibitor is an inhibitor of ITK, BTK, IL-9 (e.g., MEDI-528), IL-5 (e.g., Mepolizumab, CAS No. 196078-29-2; resilizumab), IL-13 (e.g., IMA-026, IMA-638 (also referred to as, anrukinzumab, INN No. 910649-32-0; QAX-576; IL4/IL13 trap), tralokinumab (also referred to as CAT-354, CAS No. 1044515-88-9); AER-001, ABT-308 (also referred to as humanized 13C5.5 antibody), IL-4 (e.g., AER-001, IL4/IL13 trap), IL-17, OX40L, TSLP, IL-25, IL-33 and IgE (e.g., XOLAIR®, QGE-031; MEDI-4212; quilizumab); and receptors such as: IL-9 receptor, IL-5 receptor (e.g., MEDI-563 (benralizumab, CAS No. 1044511-01-4), IL-4receptor alpha (e.g., AMG-317, AIR-645, dupilumab), IL-13receptoralpha1 (e.g., R-1671) and IL-13receptoralpha2, OX40, TSLP-R, IL-7Ralpha (a co-receptor for TSLP), IL17RB (receptor for IL-25), ST2 (receptor for IL-33), CCR3, CCR4, CRTH2 (e.g., AMG-853, AP768, AP-761, MLN6095, ACT129968), FcepsilonRI, FcepsilonRII/CD23 (receptors for IgE), Flap (e.g., GSK2190915), Syk kinase (R-343, PF3526299); CCR4 (AMG-761), TLR9 (QAX-935), or is a multi-cytokine inhibitor of CCR3, IL5, IL3, GM-CSF (e.g., TPI ASM8).

37. The method according to claim 32, wherein the Th2 pathway inhibitor is an IL-13 pathway inhibitor or an anti IgE binding agent.

38. The method according to claim 32, wherein the Th2 pathway inhibitor is an anti-IL-13 antibody or an anti-IL-13 bispecific antibody.

39. The method according to claim 38, wherein the anti-IL-13 antibody is an antibody comprising a VH comprising a sequence selected from SEQ ID NOs: 1, 3, and 24, and a VL comprising a sequence selected from SEQ ID NO: 2, 4, and 25; an anti-IL13 antibody comprising HVRH1, HVRH2, HVRH3, HVRL1, HVRL2, and HVRL3, wherein the respective HVRs have the amino acid sequence of SEQ ID NO.: 5, SEQ ID NO.: 6, SEQ ID NO.: 7, SEQ ID NO.: 8, SEQ ID NO.: 9, and SEQ ID NO.: 10; or lebrikizumab.

40-43. (canceled)

44. The method according to claim 37, wherein the Th2 pathway inhibitor is an anti-IgE antibody.

45. The method according to claim 44, wherein the anti-IgE antibody is (i) the XOLAIR® antibody or (ii) an anti-IgE antibody comprising a variable heavy chain region and a variable light chain region, wherein the variable heavy chain region is SEQ ID NO:22 and the variable light chain region is SEQ ID NO:23.

46. The method according to claim 19, wherein the patient is suffering from moderate to severe asthma.

47. The method according to claim 19, wherein the asthma or Th2-associated disease is uncontrolled on a corticosteroid.

48. The method according to claim 47, wherein the corticosteroid is an inhaled corticosteroid.

49. The method according to claim 46, wherein the patient is being treated with a second controller.

50. The method according to claim 19, wherein the patient is a human.

51. The method according to claim 19, wherein the sample is serum or plasma.

52-71. (canceled)

72. A kit for stratifying an asthma patient or a Th2-associated disease patient wherein the kit comprises:
a) reagents for measuring the IL-13 level in a sample obtained from the patient; and
b) instructions for (i) measuring the IL-13 level according to the method of claim 1, (ii) comparing the measured IL-13 level to a reference level, and (iii) stratifying said patient into the category of responder or non-responder based on the comparison.

73. The kit according to claim 72, wherein the kit comprises a package insert for determining whether the patient is likely to respond to a Th2 pathway inhibitor.

74. (canceled)

75. The kit according to claim 72, further comprising an empty container to hold a biological sample.

76. A method of identifying an asthma patient as likely to suffer from severe exacerbations, the method comprising:
obtaining a sample from the patient,
measuring the level of IL-13 in the sample according to the method of claim 1, comparing the IL-13 level detected in the sample to a reference level, and
predicting that the patient is likely to suffer from severe exacerbations when the IL-13 level measured in the sample is elevated compared to the reference level.

77. The method of claim 76, wherein the reference level is the median level of IL-13 in a reference population.

78. The method of claim 19 or 76, further comprising measuring the level of one or more Th2-associated biomarkers selected from periostin, FeNO, eosinophils, and IgE.

79. (canceled)

80. The method of claim 19, wherein the Th2-associated disease is selected from asthma, atopic dermatitis, idiopathic pulmonary fibrosis, allergic rhinitis, fibrosis, inflammatory bowel disease, ulcerative colitis, Crohn's disease, chronic obstructive pulmonary disease, and hepatic fibrosis.

81. (canceled)

82. The method according to claim 39, wherein the administering step comprises administering 37.5 mg of the anti-IL-13 antibody every four weeks or 125 mg of the anti-IL-13 antibody every four weeks.

83. The method according to claim 82, further comprising measuring the level of one or more Th2-associated biomarkers selected from periostin, FeNO, eosinophils, and IgE.

84. The method according to claim 83, wherein the Th2-associated biomarker is blood eosinophils.

85. The method of claim 84, wherein the level of blood eosinophils is determined as 300 cells/microliter or above.


SEQUENCE LISTING (33 sequences)

SEQ ID NO: 1 - protein, 117 aa, Artificial Sequence
  source: /note=“Description of Artificial Sequence Synthetic polypeptide”
VTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSS

SEQ ID NO: 2 - protein, 111 aa, Artificial Sequence [sequence omitted]

SEQ ID NO: 3 - protein, 118 aa, Artificial Sequence [sequence omitted]

SEQ ID NO: 4 - protein, 112 aa, Artificial Sequence
  source: /note=“Description of Artificial Sequence Synthetic polypeptide”
DIVMTQSPDSLSVSLGERATINCRASKSVDSYGNSFMHWYQQKPGQPPKLLIYLASNLESGVPDRFSGSGSGTDFTLTISSLQAEDVAVYYCQQNNEDPRTFGGGTKVEIKR

SEQ ID NO: 5 - protein, 5 aa, Artificial Sequence
  source: /note=“Description of Artificial Sequence Synthetic peptide”
AYSVN

SEQ ID NO: 6 - protein, 16 aa, Artificial Sequence
  source: /note=“Description of Artificial Sequence Synthetic peptide”
MIWGDGKIVYNSALKS

SEQ ID NO: 7 - protein, 10 aa, Artificial Sequence
  source: /note=“Description of Artificial Sequence Synthetic peptide”
DGYYPYAMDN

SEQ ID NO: 8 - protein, 15 aa, Artificial Sequence
  source: /note=“Description of Artificial Sequence Synthetic peptide”
RASKSVDSYGNSFMH

SEQ ID NO: 9 - protein, 7 aa, Artificial Sequence
  source: /note=“Description of Artificial Sequence Synthetic peptide”
LASNLES

SEQ ID NO: 10 - protein, 9 aa, Artificial Sequence
  source: /note=“Description of Artificial Sequence Synthetic peptide”
QQNNEDPRT

SEQ ID NO: 11 - protein, 106 aa, Artificial Sequence
  source: /note=“Description of Artificial Sequence Synthetic polypeptide”
DIVLTQSPASLAVSLGQRATISCRASQSVSTSSYSYMNWYQQTPGQPPKLLIKYASNLESGIPARFSGSGSGTDFTLNIHPVEEEDTATYYCQHSWEIPYTFGGGT

SEQ ID NO: 12 - protein, 123 aa, Artificial Sequence
  source: /note=“Description of Artificial Sequence Synthetic polypeptide”
QVTLKESGPGILQPSQTLSLTCSFSGFSLSTSDMGVGWIRQPSGKGLEWLAHIWWDDVKRYNPALKSRLTISKDTSSSQVFLKIASVDTADTATYYCARIGTNYGYDGLFDYWGQGTTLTVSS

SEQ ID NO: 13 - protein, 12 aa, Artificial Sequence
  source: /note=“Description of Artificial Sequence Synthetic peptide”
GFSLSTSDMGVG

SEQ ID NO: 14 - protein, 17 aa, Artificial Sequence
  source: /note=“Description of Artificial Sequence Synthetic peptide”
AHIWWDDVKRYNPALKS

SEQ ID NO: 15 - protein, 15 aa, Artificial Sequence
  source: /note=“Description of Artificial Sequence Synthetic peptide”
ARIGTNYGYDGLFDY

SEQ ID NO: 16 - protein, 15 aa, Artificial Sequence
  source: /note=“Description of Artificial Sequence Synthetic peptide”
RASQSVSTSSYSYMN

SEQ ID NO: 17 - protein, 7 aa, Artificial Sequence
  source: /note=“Description of Artificial Sequence Synthetic peptide”
YASNLES

SEQ ID NO: 18 - protein, 9 aa, Artificial Sequence
  source: /note=“Description of Artificial Sequence Synthetic peptide”
QHSWEIPYT

SEQ ID NO: 19 - protein, 114 aa, Homo sapiens
SPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 20 - protein, 8 aa, Homo sapiens
ESLINVSG

SEQ ID NO: 21 - protein, 12 aa, Homo sapiens
YCAALESLINVS

SEQ ID NO: 22 - protein, 114 aa, Artificial Sequence
  source: /note=“Description of Artificial Sequence Synthetic polypeptide”
DIQLTQSPSSLSASVGDRVTITCRASQSVDYDGDSYMNWYQQKPGKAPKLLIYAASYLESGVPSRFSGSGSGTDFTLTISSLQPEDFATYYCQQSHEDPYTFGQGTKVEIKRTV

SEQ ID NO: 23 - protein, 114 aa, Artificial Sequence
  source: /note=“Description of Artificial Sequence Synthetic polypeptide”
EVQLVESGGGLVQPGGSLRLSCAVSGYSITSGYSWNWIRQAPGKGLEWVASITYDGSTNYNPSVKGRITISRDDSKNTFYLQMNSLRAEDTAVYYCARGSHYFGHWHFAVWGQG

SEQ ID NO: 24 - protein, 118 aa, Artificial Sequence
  source: /note=“Description of Artificial Sequence Synthetic polypeptide”
EVTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSS

SEQ ID NO: 25 - protein, 112 aa, Artificial Sequence
  source: /note=“Description of Artificial Sequence Synthetic polypeptide”
DIVLTQSPDSLSVSLGERATINCRASKSVDSYGNSFMHWYQQKPGQPPKLLIYLASNLESGVPDRFSGSGSGTDFTLTISSLQAEDVAVYYCQQNNEDPRTFGGGTKVEIKR

SEQ ID NO: 26 - protein, 5 aa, Artificial Sequence
  source: /note=“Description of Artificial Sequence Synthetic peptide”
DYAMH

SEQ ID NO: 27 - protein, 17 aa, Artificial Sequence
  source: /note=“Description of Artificial Sequence Synthetic peptide”
GINWSSGGIGYADSVKG

SEQ ID NO: 28 - protein, 14 aa, Artificial Sequence
  source: /note=“Description of Artificial Sequence Synthetic peptide”
DIGGFGEFYWNFGL

SEQ ID NO: 29 - protein, 11 aa, Artificial Sequence
  source: /note=“Description of Artificial Sequence Synthetic peptide”
RASQSVRSYLA

SEQ ID NO: 30 - protein, 7 aa, Artificial Sequence
  source: /note=“Description of Artificial Sequence Synthetic peptide”
DASNRAT

SEQ ID NO: 31 - protein, 10 aa, Artificial Sequence
  source: /note=“Description of Artificial Sequence Synthetic peptide”
QQRSNWPPAT

SEQ ID NO: 32 - protein, 123 aa, Artificial Sequence
  source: /note=“Description of Artificial Sequence Synthetic polypeptide”
EVQLVESGGGLVQPGRSLRLSCAASGFTFDDYAMHWVRQAPGKGLEWVSGINWSSGGIGYADSVKGRFTISRDNAKNSLYLQMNSLRAEDTALYYCARDIGGFGEFYWNFGLWGRGTLVTVSS

SEQ ID NO: 33 - protein, 108 aa, Artificial Sequence
  source: /note=“Description of Artificial Sequence Synthetic polypeptide”
EIVLTQSPATLSLSPGERATLSCRASQSVRSYLAWYQQKPGQAPRLLIYDASNRATGIPARFSGSGSGTDFTLTISSLEPEDFAVYYCQQRSNWPPATFGGGTKVEIK